SEQUENCE LISTING (274 sequences)

SEQ ID NO: 1 - RNA, 22 nt, Homo sapiens
uggugguuuacaaaguaauuca

SEQ ID NO: 2 - RNA, 21 nt, Homo sapiens
uacaguacugugauaacugaa

SEQ ID NO: 3 - RNA, 22 nt, Homo sapiens
ugucuuacucccucaggcacau

SEQ ID NO: 4 - RNA, 22 nt, Homo sapiens
aaccagcaccccaacuuuggac

SEQ ID NO: 5 - RNA, 22 nt, Homo sapiens
ugugacagauugauaacugaaa

SEQ ID NO: 6 - RNA, 23 nt, Homo sapiens
agcuucuuuacagugcugccuug

SEQ ID NO: 7 - RNA, 22 nt, Homo sapiens
uggugggccgcagaacaugugc

SEQ ID NO: 8 - RNA, 23 nt, Homo sapiens
uggaguccaggaaucugcauuuu

SEQ ID NO: 9 - RNA, 22 nt, Homo sapiens
auucugcauuuuuagcaaguuc

SEQ ID NO: 10 - RNA, 22 nt, Homo sapiens
cacuggcuccuuucuggguaga

SEQ ID NO: 11 - RNA, 23 nt, Homo sapiens
aguuuugcagguuugcauccagc

SEQ ID NO: 12 - RNA, 22 nt, Homo sapiens
gugacaucacauauacggcagc

SEQ ID NO: 13 - RNA, 22 nt, Homo sapiens
cugccaauuccauaggucacag

SEQ ID NO: 14 - RNA, 22 nt, Homo sapiens
aaguucuguuauacacucaggc

SEQ ID NO: 15 - RNA, 21 nt, Homo sapiens
aacaggugacugguuagacaa

SEQ ID NO: 16 - RNA, 22 nt, Homo sapiens
cuuaugcaagauucccuucuac

SEQ ID NO: 17 - RNA, 20 nt, Homo sapiens
gugucugggcggacagcugc

SEQ ID NO: 18 - RNA, 22 nt, Homo sapiens
aucgugcauccuuuuagagugu

SEQ ID NO: 19 - RNA, 22 nt, Homo sapiens
aucgugcaucccuuuagagugu

SEQ ID NO: 20 - RNA, 22 nt, Homo sapiens
augggugaauuuguagaaggau

SEQ ID NO: 21 - RNA, 22 nt, Homo sapiens
ugguugaccauagaacaugcgc

SEQ ID NO: 22 - RNA, 22 nt, Homo sapiens
aggcauugacuucucacuagcu

SEQ ID NO: 23 - RNA, 21 nt, Homo sapiens
acggugcuggauguggccuuu

SEQ ID NO: 24 - RNA, 23 nt, Homo sapiens
agguugggaucgguugcaaugcu

SEQ ID NO: 25 - RNA, 21 nt, Homo sapiens
agagaagaagaucagccugca

SEQ ID NO: 26 - RNA, 20 nt, Homo sapiens
ccucugggcccuuccuccag

SEQ ID NO: 27 - RNA, 23 nt, Homo sapiens
uauggcuuuucauuccuauguga

SEQ ID NO: 28 - RNA, 21 nt, Homo sapiens
agggggaaaguucuauagucc

SEQ ID NO: 29 - RNA, 22 nt, Homo sapiens
cggguggaucacgaugcaauuu

SEQ ID NO: 30 - RNA, 21 nt, Homo sapiens
aucauagaggaaaauccacgu

SEQ ID NO: 31 - RNA, 21 nt, Homo sapiens
ugcaggaccaagaugagcccu

SEQ ID NO: 32 - RNA, 20 nt, Homo sapiens
auggagauagauauagaaau

SEQ ID NO: 33 - RNA, 21 nt, Homo sapiens
uaaggcacccuucugaguaga

SEQ ID NO: 34 - RNA, 20 nt, Homo sapiens
cugcaaagggaagcccuuuc

SEQ ID NO: 35 - RNA, 22 nt, Homo sapiens
aguauucuguaccagggaaggu

SEQ ID NO: 36 - RNA, 22 nt, Homo sapiens
guucucccaacguaagcccagc

SEQ ID NO: 37 - RNA, 22 nt, Homo sapiens
caugccuugaguguaggaccgu

SEQ ID NO: 38 - RNA, 21 nt, Homo sapiens
cugaccuaugaauugacagcc

SEQ ID NO: 39 - RNA, 17 nt, Homo sapiens
gugggggagaggcuguc

SEQ ID NO: 40 - RNA, 22 nt, Homo sapiens
aacuggcccucaaagucccgcu

SEQ ID NO: 41 - RNA, 21 nt, Homo sapiens
cuguacaggccacugccuugc

SEQ ID NO: 42 - RNA, 23 nt, Homo sapiens
uauggcuuuuuauuccuauguga

SEQ ID NO: 43 - RNA, 22 nt, Homo sapiens
ugccugucuacacuugcugugc

SEQ ID NO: 44 - RNA, 24 nt, Homo sapiens
acugggggcuuucgggcucugcgu

SEQ ID NO: 45 - RNA, 21 nt, Homo sapiens
aggguaagcugaaccucugau

SEQ ID NO: 46 - RNA, 21 nt, Homo sapiens
aggggugcuaucugugauuga

SEQ ID NO: 47 - RNA, 22 nt, Homo sapiens
aacacaccuauucaaggauuca

SEQ ID NO: 48 - RNA, 21 nt, Homo sapiens
uccgguucucagggcuccacc

SEQ ID NO: 49 - RNA, 24 nt, Homo sapiens
agccugauuaaacacaugcucuga

SEQ ID NO: 50 - RNA, 22 nt, Homo sapiens
caggccauauugugcugccuca

SEQ ID NO: 51 - RNA, 22 nt, Homo sapiens
aacuggccuacaaagucccagu

SEQ ID NO: 52 - RNA, 19 nt, Homo sapiens
aggcugcggaauucaggac

SEQ ID NO: 53 - RNA, 18 nt, Homo sapiens
acguuggcucugguggug

SEQ ID NO: 54 - RNA, 23 nt, Homo sapiens
aaggagcuuacaaucuagcuggg

SEQ ID NO: 55 - RNA, 22 nt, Homo sapiens
uugggaucauuuugcauccaua

SEQ ID NO: 56 - RNA, 22 nt, Homo sapiens
guagauucuccuucuaugagua

SEQ ID NO: 57 - RNA, 22 nt, Homo sapiens
aggggcuggcuuuccucugguc

SEQ ID NO: 58 - RNA, 22 nt, Homo sapiens
uggugggcacagaaucuggacu

SEQ ID NO: 59 - RNA, 22 nt, Homo sapiens
gauuagggugcuuagcuguuaa

SEQ ID NO: 60 - RNA, 19 nt, Homo sapiens
ugucucugcugggguuucu

SEQ ID NO: 61 - RNA, 22 nt, Homo sapiens
ugccuacugagcugaaacacag

SEQ ID NO: 62 - RNA, 21 nt, Homo sapiens
cacaagguauugguauuaccu

SEQ ID NO: 63 - RNA, 20 nt, Homo sapiens
accaggaggcugaggccccu

SEQ ID NO: 64 - RNA, 22 nt, Homo sapiens
caucaucgucucaaaugagucu

SEQ ID NO: 65 - RNA, 23 nt, Homo sapiens
cuggagauauggaagagcugugu

SEQ ID NO: 66 - RNA, 21 nt, Homo sapiens
ugucuugcaggccgucaugca

SEQ ID NO: 67 - RNA, 22 nt, Homo sapiens
cucaucugcaaagaaguaagug

SEQ ID NO: 68 - RNA, 20 nt, Homo sapiens
agagguauagggcaugggaa

SEQ ID NO: 69 - RNA, 23 nt, Homo sapiens
uuuggcacuagcacauuuuugcu

SEQ ID NO: 70 - RNA, 22 nt, Homo sapiens
uggagagaaaggcaguuccuga

SEQ ID NO: 71 - RNA, 22 nt, Homo sapiens
ugcggggcuagggcuaacagca

SEQ ID NO: 72 - RNA, 22 nt, Homo sapiens
uuauaaagcaaugagacugauu

SEQ ID NO: 73 - RNA, 21 nt, Homo sapiens
guggcugcacucacuuccuuc

SEQ ID NO: 74 - RNA, 21 nt, Homo sapiens
aaggcagggcccccgcucccc

SEQ ID NO: 75 - RNA, 21 nt, Homo sapiens
uuaggccgcagaucuggguga

SEQ ID NO: 76 - RNA, 22 nt, Homo sapiens
ugugagguuggcauuguugucu

SEQ ID NO: 77 - RNA, 22 nt, Homo sapiens
auguagggcuaaaagccauggg

SEQ ID NO: 78 - RNA, 25 nt, Homo sapiens
aggggugguguugggacagcuccgu

SEQ ID NO: 79 - RNA, 22 nt, Homo sapiens
uuggggaaacggccgcugagug

SEQ ID NO: 80 - RNA, 21 nt, Homo sapiens
cagcagcacacugugguuugu

SEQ ID NO: 81 - RNA, 22 nt, Homo sapiens
uucaccaccuucuccacccagc

SEQ ID NO: 82 - RNA, 21 nt, Homo sapiens
cuucuugugcucuaggauugu

SEQ ID NO: 83 - RNA, 21 nt, Homo sapiens
agaccuggcccagaccucagc

SEQ ID NO: 84 - RNA, 22 nt, Homo sapiens
ugcaacuuaccugagucauuga

SEQ ID NO: 85 - RNA, 20 nt, Homo sapiens
aggguguuucucucaucucu

SEQ ID NO: 86 - RNA, 22 nt, Homo sapiens
aucuggagguaagaagcacuuu

SEQ ID NO: 87 - RNA, 22 nt, Homo sapiens
cucaguagccaguguagauccu

SEQ ID NO: 88 - RNA, 22 nt, Homo sapiens
augguacccuggcauacugagu

SEQ ID NO: 89 - RNA, 22 nt, Homo sapiens
caucuuccaguacaguguugga

SEQ ID NO: 90 - RNA, 22 nt, Homo sapiens
ugagaccucuggguucugagcu

SEQ ID NO: 91 - RNA, 23 nt, Homo sapiens
ucaaaugcucagacuccuguggu

SEQ ID NO: 92 - RNA, 22 nt, Homo sapiens
agggcuuagcugcuugugagca

SEQ ID NO: 93 - RNA, 22 nt, Homo sapiens
acuguugcuaauaugcaacucu

SEQ ID NO: 94 - RNA, 22 nt, Homo sapiens
aguggggaacccuuccaugagg

SEQ ID NO: 95 - RNA, 22 nt, Homo sapiens
acacagggcuguugugaagacu

SEQ ID NO: 96 - RNA, 22 nt, Homo sapiens
cuauacagucuacugucuuucc

SEQ ID NO: 97 - RNA, 22 nt, Homo sapiens
aaaaguacuugcggauuuugcu

SEQ ID NO: 98 - RNA, 22 nt, Homo sapiens
agacuucccauuugaagguggc

SEQ ID NO: 99 - RNA, 21 nt, Homo sapiens
cggggcagcucaguacaggau

SEQ ID NO: 100 - RNA, 21 nt, Homo sapiens
uggaggagaaggaaggugaug

SEQ ID NO: 101 - RNA, 21 nt, Homo sapiens
ugguucuagacuugccaacua

SEQ ID NO: 102 - RNA, 26 nt, Homo sapiens
aaguaguugguuuguaugagaugguu

SEQ ID NO: 103 - RNA, 21 nt, Homo sapiens
augaccuaugaauugacagac

SEQ ID NO: 104 - RNA, 19 nt, Homo sapiens
aguguggcuuucuuagagc

SEQ ID NO: 105 - RNA, 22 nt, Homo sapiens
cguggauauuccuucuauguuu

SEQ ID NO: 106 - RNA, 20 nt, Homo sapiens
ucugcaggguuugcuuugag

SEQ ID NO: 107 - RNA, 22 nt, Homo sapiens
ucucugggccugugucuuaggc

SEQ ID NO: 108 - RNA, 21 nt, Homo sapiens
cuggauggcuccuccaugucu

SEQ ID NO: 109 - RNA, 22 nt, Homo sapiens
cuuggcaccuagcaagcacuca

SEQ ID NO: 110 - RNA, 22 nt, Homo sapiens
aggcagcgggguguaguggaua

SEQ ID NO: 111 - RNA, 18 nt, Homo sapiens
ugaggcaguagauugaau

SEQ ID NO: 112 - RNA, 22 nt, Homo sapiens
aguucuucaguggcaagcuuua

SEQ ID NO: 113 - RNA, 22 nt, Homo sapiens
aaauuauuguacaucggaugag

SEQ ID NO: 114 - RNA, 25 nt, Homo sapiens
gcugggcagggcuucugagcuccuu

SEQ ID NO: 115 - RNA, 23 nt, Homo sapiens
ucauagcccuguacaaugcugcu

SEQ ID NO: 116 - RNA, 22 nt, Homo sapiens
agaguugagucuggacgucccg

SEQ ID NO: 117 - RNA, 22 nt, Homo sapiens
caguuaucacagugcugaugcu

SEQ ID NO: 118 - RNA, 22 nt, Homo sapiens
ucugggcaacaaagugagaccu

SEQ ID NO: 119 - RNA, 22 nt, Homo sapiens
uggguggucuggagauuugugc

SEQ ID NO: 120 - RNA, 22 nt, Homo sapiens
aacaucacagcaagucugugcu

SEQ ID NO: 121 - RNA, 22 nt, Homo sapiens
auaagacgagcaaaaagcuugu

SEQ ID NO: 122 - RNA, 21 nt, Homo sapiens
agaggauacccuuuguauguu

SEQ ID NO: 123 - RNA, 21 nt, Homo sapiens
uugggacauacuuaugcuaaa

SEQ ID NO: 124 - RNA, 22 nt, Homo sapiens
uagcagcacguaaauauuggcg

SEQ ID NO: 125 - RNA, 22 nt, Homo sapiens
aaggagcucacagucuauugag

SEQ ID NO: 126 - RNA, 22 nt, Homo sapiens
cuccugacuccagguccugugu

SEQ ID NO: 127 - RNA, 22 nt, Homo sapiens
uauggcacugguagaauucacu

SEQ ID NO: 128 - RNA, 22 nt, Homo sapiens
aacacaccugguuaaccucuuu

SEQ ID NO: 129 - RNA, 21 nt, Homo sapiens
ugaguuggccaucugagugag

SEQ ID NO: 130 - RNA, 22 nt, Homo sapiens
ugagguaguaguuuguacaguu

SEQ ID NO: 131 - RNA, 22 nt, Homo sapiens
gcaggugcucacuuguccuccu

SEQ ID NO: 132 - RNA, 22 nt, Homo sapiens
augcugacauauuuacuagagg

SEQ ID NO: 133 - RNA, 18 nt, Homo sapiens
uugagaaggaggcugcug

SEQ ID NO: 134 - RNA, 22 nt, Homo sapiens
caucagaauucauggaggcuag

SEQ ID NO: 135 - RNA, 22 nt, Homo sapiens
uacucaggagaguggcaaucac

SEQ ID NO: 136 - RNA, 22 nt, Homo sapiens
cgcaggggccgggugcucaccg

SEQ ID NO: 137 - RNA, 19 nt, Homo sapiens
gaugaugcugcugaugcug

SEQ ID NO: 138 - RNA, 21 nt, Homo sapiens
gugucuuuugcucugcaguca

SEQ ID NO: 139 - RNA, 20 nt, Homo sapiens
auauggguuuacuaguuggu

SEQ ID NO: 140 - RNA, 22 nt, Homo sapiens
ugccuggaacauaguagggacu

SEQ ID NO: 141 - RNA, 20 nt, Homo sapiens
uggcuuuuaacuuugauggc

SEQ ID NO: 142 - RNA, 22 nt, Homo sapiens
acugcugagcuagcacuucccg

SEQ ID NO: 143 - RNA, 20 nt, Homo sapiens
uagaggaagcuguggagaga

SEQ ID NO: 144 - RNA, 23 nt, Homo sapiens
aucagggcuuguggaaugggaag

SEQ ID NO: 145 - RNA, 22 nt, Homo sapiens
aggugguccguggcgcguucgc

SEQ ID NO: 146 - RNA, 19 nt, Homo sapiens
aagcagcugccucugaggc

SEQ ID NO: 147 - RNA, 22 nt, Homo sapiens
aggcaguguauuguuagcuggc

SEQ ID NO: 148 - RNA, 22 nt, Homo sapiens
aaccaucgaccguugaguggac

SEQ ID NO: 149 - RNA, 22 nt, Homo sapiens
ccaguauuaacugugcugcuga

SEQ ID NO: 150 - RNA, 22 nt, Homo sapiens
cuguugccacuaaccucaaccu

SEQ ID NO: 151 - RNA, 19 nt, Homo sapiens
ugggcguaucuguaugcua

SEQ ID NO: 152 - RNA, 23 nt, Homo sapiens
uaggcagugucauuagcugauug

SEQ ID NO: 153 - RNA, 22 nt, Homo sapiens
acuccagccccacagccucagc

SEQ ID NO: 154 - RNA, 20 nt, Homo sapiens
guguguggaaaugcuucugc

SEQ ID NO: 155 - RNA, 23 nt, Homo sapiens
uaggagcucaacagaugccuguu

SEQ ID NO: 156 - RNA, 22 nt, Homo sapiens
agcuuuugggaauucagguagu

SEQ ID NO: 157 - RNA, 22 nt, Homo sapiens
ucguaccgugaguaauaaugcg

SEQ ID NO: 158 - RNA, 22 nt, Homo sapiens
accuggcauacaauguagauuu

SEQ ID NO: 159 - RNA, 19 nt, Homo sapiens
auggauaaggcuuuggcuu

SEQ ID NO: 160 - RNA, 23 nt, Homo sapiens
aucaacagacauuaauugggcgc

SEQ ID NO: 161 - RNA, 22 nt, Homo sapiens
auauaccuguucggucucuuua

SEQ ID NO: 162 - RNA, 22 nt, Homo sapiens
gacugacaccucuuugggugaa

SEQ ID NO: 163 - RNA, 21 nt, Homo sapiens
uggguuuacguugggagaacu

SEQ ID NO: 164 - RNA, 22 nt, Homo sapiens
aagauguggaaaaauuggaauc

SEQ ID NO: 165 - RNA, 21 nt, Homo sapiens
aggcggagacuugggcaauug

SEQ ID NO: 166 - RNA, 22 nt, Homo sapiens
cuggggagauccucgagguugg

SEQ ID NO: 167 - RNA, 21 nt, Homo sapiens
cuuuuugcggucugggcuugc

SEQ ID NO: 168 - RNA, 22 nt, Homo sapiens
cagaaggggaguugggagcaga

SEQ ID NO: 169 - RNA, 19 nt, Homo sapiens
ggccuuguuccugucccca

SEQ ID NO: 170 - RNA, 22 nt, Homo sapiens
uucagccaggcuagugcagucu

SEQ ID NO: 171 - RNA, 22 nt, Homo sapiens
aagggcuuccucucugcaggac

SEQ ID NO: 172 - RNA, 18 nt, Homo sapiens
ccgcuuucugagcuggac

SEQ ID NO: 173 - RNA, 22 nt, Homo sapiens
agagcugagacuagaaagccca

SEQ ID NO: 174 - RNA, 18 nt, Homo sapiens
cucugggaaaugggacag

SEQ ID NO: 175 - RNA, 22 nt, Homo sapiens
uguguggauccuggaggaggca

SEQ ID NO: 176 - RNA, 23 nt, Homo sapiens
cgagggcauuucaugaugcaggc

SEQ ID NO: 177 - RNA, 22 nt, Homo sapiens
ugucuacauuaaugaaaagagc

SEQ ID NO: 178 - RNA, 22 nt, Homo sapiens
ucagcaggcaggcuggugcagc

SEQ ID NO: 179 - RNA, 26 nt, Homo sapiens
aagaggaagaaauggcugguucucag

SEQ ID NO: 180 - RNA, 21 nt, Homo sapiens
acagggccgcagauggagacu

SEQ ID NO: 181 - RNA, 21 nt, Homo sapiens
ugaggagaucgucgagguugg

SEQ ID NO: 182 - RNA, 22 nt, Homo sapiens
agguggaugcaaugugaccuca

SEQ ID NO: 183 - RNA, 23 nt, Homo sapiens
cgcagacaaugccuacuggccua

SEQ ID NO: 184 - RNA, 18 nt, Homo sapiens
aucugaccugaugaaggu

SEQ ID NO: 185 - RNA, 22 nt, Homo sapiens
cugggguucugagacagacagu

SEQ ID NO: 186 - RNA, 22 nt, Homo sapiens
caguugggucuaggggucagga

SEQ ID NO: 187 - RNA, 22 nt, Homo sapiens
aaaggaggaaauaggcaggcca

SEQ ID NO: 188 - RNA, 23 nt, Homo sapiens
gggaugguagaccggugacgugc

SEQ ID NO: 189 - RNA, 20 nt, Homo sapiens
aggcaugggaggucagguga

SEQ ID NO: 190 - RNA, 22 nt, Homo sapiens
aacuaguaauguuggauuaggg

SEQ ID NO: 191 - RNA, 22 nt, Homo sapiens
cagguagauauuugauaggcau

SEQ ID NO: 192 - RNA, 23 nt, Homo sapiens
uagugaguuagagaugcagagcc

SEQ ID NO: 193 - RNA, 18 nt, Homo sapiens
uugcacuugucucaguga

SEQ ID NO: 194 - RNA, 22 nt, Homo sapiens
agaaggggugaaauuuaaacgu

SEQ ID NO: 195 - RNA, 22 nt, Homo sapiens
uggggcggagcuuccggaggcc

SEQ ID NO: 196 - RNA, 19 nt, Homo sapiens
cugugggcucagcucuggg

SEQ ID NO: 197 - RNA, 18 nt, Homo sapiens
cuaagaaguugacugaag

SEQ ID NO: 198 - RNA, 22 nt, Homo sapiens
uaagggguguauggcagaugca

SEQ ID NO: 199 - RNA, 21 nt, Homo sapiens
ggcuccuccucucaggaugug

SEQ ID NO: 200 - RNA, 21 nt, Homo sapiens
ucacgcggagagauggcuuug

SEQ ID NO: 201 - RNA, 20 nt, Homo sapiens
ucagggagucaggggagggc

SEQ ID NO: 202 - RNA, 22 nt, Homo sapiens
ugaccugggacucggacagcug

SEQ ID NO: 203 - RNA, 18 nt, Homo sapiens
agcaggugcggggcggcg

SEQ ID NO: 204 - RNA, 18 nt, Homo sapiens
aaagcuggguugagaagg

SEQ ID NO: 205 - RNA, 17 nt, Homo sapiens
cucagugacucaugugc

SEQ ID NO: 206 - RNA, 23 nt, Homo sapiens
uguggaagguagacggccagaga

SEQ ID NO: 207 - RNA, 23 nt, Homo sapiens
ucugggagguuguagcaguggaa

SEQ ID NO: 208 - RNA, 23 nt, Homo sapiens
augagacucauguaaaacaucuu

SEQ ID NO: 209 - RNA, 23 nt, Homo sapiens
uauggggcuucuguagagauuuc

SEQ ID NO: 210 - RNA, 17 nt, Homo sapiens
uggggcucagcgaguuu

SEQ ID NO: 211 - RNA, 22 nt, Homo sapiens
caccuugcgcuacucaggucug

SEQ ID NO: 212 - RNA, 19 nt, Homo sapiens
ugcccuccuuucuucccuc

SEQ ID NO: 213 - RNA, 22 nt, Homo sapiens
caucuggcauccgucacacaga

SEQ ID NO: 214 - RNA, 21 nt, Homo sapiens
ugaugauacagguggagguag

SEQ ID NO: 215 - RNA, 16 nt, Homo sapiens
ugccuuccugucugug

SEQ ID NO: 216 - RNA, 23 nt, Homo sapiens
ugaggauggauagcaaggaagcc

SEQ ID NO: 217 - RNA, 17 nt, Homo sapiens
uggagagaaaggcagua

SEQ ID NO: 218 - RNA, 19 nt, Homo sapiens
uggggcggagcuuccggag

SEQ ID NO: 219 - RNA, 22 nt, Homo sapiens
aggugcuccaggcuggcucaca

SEQ ID NO: 220 - RNA, 18 nt, Homo sapiens
cagggaggugaaugugau

SEQ ID NO: 221 - RNA, 22 nt, Homo sapiens
cacccguagaaccgaccuugcg

SEQ ID NO: 222 - RNA, 22 nt, Homo sapiens
aacccguagauccgaacuugug

SEQ ID NO: 223 - RNA, 21 nt, Homo sapiens
gguggggcaaugggaucaggu

SEQ ID NO: 224 - RNA, 19 nt, Homo sapiens
aucgggcccucggcgccgg

SEQ ID NO: 225 - RNA, 22 nt, Homo sapiens
uggcagugucuuagcugguugu

SEQ ID NO: 226 - RNA, 23 nt, Homo sapiens
aggcaguguaguuagcugauugc

SEQ ID NO: 227 - RNA, 22 nt, Homo sapiens
aaaaugaaaugagcccagccca

SEQ ID NO: 228 - RNA, 22 nt, Homo sapiens
uauguaauaugguccacaucuu

SEQ ID NO: 229 - RNA, 22 nt, Homo sapiens
auauguauaugugacugcuacu

SEQ ID NO: 230 - RNA, 22 nt, Homo sapiens
uggcaguguauuguuagcuggu

SEQ ID NO: 231 - RNA, 22 nt, Homo sapiens
gucauacacggcucuccucucu

SEQ ID NO: 232 - RNA, 22 nt, Homo sapiens
gagugccuucuuuuggagcguu

SEQ ID NO: 233 - RNA, 22 nt, Homo sapiens
uaugugggaugguaaaccgcuu

SEQ ID NO: 234 - RNA, 22 nt, Homo sapiens
cuauacaaccuacugccuuccc

SEQ ID NO: 235 - RNA, 22 nt, Homo sapiens
acaggugagguucuugggagcc

SEQ ID NO: 236 - RNA, 22 nt, Homo sapiens
ucucccaacccuuguaccagug

SEQ ID NO: 237 - RNA, 21 nt, Homo sapiens
ugagugccggugccugcccug

SEQ ID NO: 238 - RNA, 21 nt, Homo sapiens
ugauauguuugauauuggguu

SEQ ID NO: 239 - RNA, 23 nt, Homo sapiens
ugaguaccgccaugucuguuggg

SEQ ID NO: 240 - RNA, 21 nt, Homo sapiens
gguuugguccuagccuuucua

SEQ ID NO: 241 - RNA, 23 nt, Homo sapiens
cccaguguuuagacuaucuguuc

SEQ ID NO: 242 - RNA, 21 nt, Homo sapiens
ccucccaugccaagaacuccc

SEQ ID NO: 243 - RNA, 22 nt, Homo sapiens
aaaucucugcaggcaaauguga

SEQ ID NO: 244 - RNA, 22 nt, Homo sapiens
cugguuucacaugguggcuuag

SEQ ID NO: 245 - RNA, 23 nt, Homo sapiens
uaagugcuuccauguuuuaguag

SEQ ID NO: 246 - RNA, 24 nt, Homo sapiens
ucuguagccugggagcaauggggu

SEQ ID NO: 247 - RNA, 24 nt, Homo sapiens
gguugggcagugaggaggguguga

SEQ ID NO: 248 - RNA, 23 nt, Homo sapiens
cugauaagaacagaggcccagau

SEQ ID NO: 249 - RNA, 23 nt, Homo sapiens
uuagggaguagaaggguggggag

SEQ ID NO: 250 - RNA, 22 nt, Homo sapiens
ugugacuuuaagggaaauggcg

SEQ ID NO: 251 - RNA, 22 nt, Homo sapiens
cggggagagaacgcagugacgu

SEQ ID NO: 252 - RNA, 23 nt, Homo sapiens
agggacugccuuaggagaaaguu

SEQ ID NO: 253 - RNA, 23 nt, Homo sapiens
aaagugcugcgacauuugagcgu

SEQ ID NO: 254 - RNA, 21 nt, Homo sapiens
ugguagacuauggaacguagg

SEQ ID NO: 255 - RNA, 22 nt, Homo sapiens
ucagguguggaaacugaggcag

SEQ ID NO: 256 - RNA, 21 nt, Homo sapiens
aauauaacacagauggccugu

SEQ ID NO: 257 - RNA, 17 nt, Homo sapiens
acucagucauggucauu

SEQ ID NO: 258 - RNA, 18 nt, Homo sapiens
cagcagucccucccccug

SEQ ID NO: 259 - RNA, 17 nt, Homo sapiens
augugggcucaggcuca

SEQ ID NO: 260 - RNA, 18 nt, Homo sapiens
ccaguguggcucagcgag

SEQ ID NO: 261 - RNA, 22 nt, Homo sapiens
uucucaaggaggugucguuuau

SEQ ID NO: 262 - RNA, 18 nt, Homo sapiens
ugcuuccuuucagagggu

SEQ ID NO: 263 - RNA, 23 nt, Homo sapiens
ucucuggagggaagcacuuucug

SEQ ID NO: 264 - RNA, 22 nt, Homo sapiens
aaagugcuucccuuuggacugu

SEQ ID NO: 265 - RNA, 21 nt, Homo sapiens
aaagugcuuccuuuuagaggg

SEQ ID NO: 266 - RNA, 22 nt, Homo sapiens
aaagugcuucucuuuggugggu

SEQ ID NO: 267 - RNA, 21 nt, Homo sapiens
aaagugcuuccuuuuugaggg

SEQ ID NO: 268 - RNA, 22 nt, Homo sapiens
gacuauagaacuuucccccuca

SEQ ID NO: 269 - RNA, 19 nt, Homo sapiens
aagugugcagggcacuggu

SEQ ID NO: 270 - RNA, 21 nt, Homo sapiens
aauggcgccacuaggguugug

SEQ ID NO: 271 - RNA, 21 nt, Homo sapiens
ucccacguuguggcccagcag

SEQ ID NO: 272 - RNA, 22 nt, Homo sapiens
caaagcgcuccccuuuagaggu

SEQ ID NO: 273 - RNA, 22 nt, Homo sapiens
uucuccaaaagggagcacuuuc

SEQ ID NO: 274 - RNA, 23 nt, Homo sapiens
cucuugagggaagcacuuucugu

(19) United States
(12) Patent Application Publication
(10) Pub. No.: US 2019/0300882 A1
(43) Pub. Date: Oct. 3, 2019

(54) METHODS OF TREATING NEUROBLASTOMA AND REAGENTS THEREFOR

(71) Applicants: GARVAN INSTITUTE OF MEDICAL RESEARCH, Darlinghurst, New South Wales (AU); PETER MACCALLUM CANCER INSTITUTE, Melbourne, Victoria (AU)

(72) Inventors: Alex SWARBRICK, Redfern (AU); Iva NIKOLIC, South Yarra (AU); Kaylene SIMPSON, McKinnon (AU)

(73) Assignees: GARVAN INSTITUTE OF MEDICAL RESEARCH, Darlinghurst, New South Wales (AU); PETER MACCALLUM CANCER INSTITUTE, Melbourne, Victoria (AU)

(21) Appl. No.: 16/307,374

(22) PCT Filed: Jun. 7, 2017
(86) PCT No.: PCT/AU2017/050564

§ 371 Date: Dec. 5, 2018

(30) Foreign Application Priority Data
Jun. 7, 2016 (AU) 2016902215

(51) Int. Cl. C12N 15/113; A61P 35/00; A61K 31/475; A61K 31/704; A61K 31/7105

(52) U.S. Cl. CPC C12N 15/1135; A61P 35/00; A61K 31/475; C12N 2320/31; A61K 31/7105; C12N 2310/141; A61K 31/704

(57) ABSTRACT

This disclosure relates to RNA interference (RNAi) reagents for treatment of neuroblastoma, compositions comprising same, and use thereof to treat individuals suffering from neuroblastoma as a monotherapy or in combination with a chemotherapeutic agent. In particular, the present disclosure relates to microRNAs (miRNAs) which affect viability of neuroblastoma cells.

### RELATED APPLICATION DATA

The present application claims priority from Australian Provisional Application No. 2016902215 filed on 7 Jun. 2016, the full contents of which is incorporated herein by reference.

### TECHNICAL FIELD

The present disclosure generally relates to methods and reagents for treating neuroblastoma. In particular, the present disclosure relates to the use of RNA interference (RNAi) molecules, such as miRNA and precursor molecules thereof, which effect viability of neuroblastoma cells and their use to treat neuroblastoma. For example, the present disclosure relates to the use of RNAi molecules as standalone therapeutics for treating neuroblastoma, as well as their use as adjunctive agents in combination with existing chemotherapeutic agents for treatment of neuroblastoma.

### BACKGROUND

MicroRNAs (miRNAs) are a class of cellular small noncoding RNAs that act to regulate protein synthesis through messenger RNA (mRNA) destabilisation and inhibition of translation (Bushati and Cohen 2007). They are typically synthesised through the formation of long hairpin molecules, which are then processed through a series of enzymatic steps into single-stranded RNAs, around 22 nt long (Ha et al, 2014). To become fully functional, the mature microRNAs associate with multiple RNA-binding proteins into the RNA-Induced Silencing Complex (RISC) where they exert their effects on specific mRNAs through hybridisation to partly complementary mRNA targets. Many microRNAs bind mRNA through a canonical mechanism, mediated via complementarity between the 6-8 nt seed of the microRNA with regions typically in the 3′ UTR of the mRNA. There are however numerous exceptions to this rule: seed regions are frequently not complementary, they can be found anywhere within a transcript, or be completely absent thereby dramatically expanding the number of potential targets (Hausser et al, 2014, Nat Gen). Although computational algorithms, which generally rely on the presence of a seed-match in a transcript, are still the most widely used tools for predicting genuine microRNA targets, numerous experimental techniques for identifying microRNA targets have also been developed (Cloonan et al, 2015).

It is now well established that microRNAs control a large diversity of cellular processes, from differentiation and proliferation to cell death (Bernstein et al, 2003; Yi et al, 2008; Cimmino et al, 2005), and that their deregulation can lead to many human diseases. In this context, cancer has been widely studied, as microRNAs control cancer phenotype (Lu et al. 2005) and can potentially serve as biomarkers and a novel class of cancer therapeutics (Ling et al. 2013).

Neuroblastoma is a childhood cancer of the sympathetic nervous system, which accounts for approximately 15% of all cancer deaths in children (Park et al 2010). Neuroblastoma cancer cells are undifferentiated cells with a high metastatic potential, and only a few oncogenes such as MYCN, LIN28B, or ALK have been directly linked to neuroblastoma development and maintenance (Molenaar et al 2012). Considering a strong link between the level of cellular differentiation and tumorigenesis in neuroblastoma (Shimada et al 1999), differentiation therapy using retinoic acid is becoming the standard of care for high-risk neuroblastoma (Matthay et al 2009), but the biology underlying the process of neuroblast proliferation, differentiation, and survival remains largely unexplored.

In view of the role that miRNAs play in a wide range of cellular processes, the identification of miRNAs which play a role in neuroblast proliferation, differentiation, and survival, as well as the elucidation of their corresponding targets, has the potential for the development of new therapeutics for treatment of neuroblastoma.

### SUMMARY

The present inventors performed functional screens with miRNA libraries in neuroblastoma cell lines to determine the effect of miRNAs on cellular viability of neuroblastoma cells. In so doing, the inventors have identified miRNAs with a previously unrecognised role in survival of neuroblastoma. In particular, the inventors have identified miRNAs which, when overexpressed or administered as a stand-alone treatment, are lethal to neuroblastoma cells (hereinafter referred to as ‘lethal’ miRNAs).

The inventors have also identified miRNAs which result in lethal phenotype to neuroblastoma cells when administered in combination with chemotherapy, including low dose e.g., IC30, chemotherapy (hereinafter referred to as ‘synthetic lethal’ miRNAs).

Furthermore, the inventors have developed a microRNA function database and target prediction tool and in using same have identified three factors, Rgs16, Isl1 and RbFox2, which are necessary for neuroblastoma growth and survival and which were previously unknown to associated with neuroblastoma. Thus the inventors have identified novel targets for treatment neuroblastoma.

In one example, the present disclosure provides a method for treating neuroblastoma in a subject suffering therefrom, said method comprising administering to the subject an RNA interference (RNAi) molecule selected from the group consisting of:
(i) a microRNA (miRNA) selected from the group of miRNAs set forth in Table 1 or Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii); (vii) a miRNA of any one of (i) to (iii) which has a chemical modification or a modified nucleobase; and
(viii) a nucleic acid encoding an RNAi molecule of any one of (i)-(v).

In one example, the RNAi molecule is a lethal RNAi molecule. For example, the RNAi molecule is a lethal RNAi molecule selected from the group consisting of:
(i) a miRNA selected from the group of miRNAs set forth in Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii); (vii) a miRNA of any one of (i) to (iii) which has a chemical modification or a modified nucleobase; and
(viii) a nucleic acid encoding an RNAi molecule of any one of (i)-(v).

For example, the RNAi molecule may be a lethal RNAi molecule selected from the group consisting of:
(i) a miRNA selected from the group of miRNAs set forth in rows 1-20 of Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii); (vii) a miRNA of any one of (i) to (iii) which has a chemical modification or a modified nucleobase; and
(viii) a nucleic acid encoding an RNAi molecule of any one of (i)-(v).

An exemplary lethal RNAi molecule is a miRNA selected from the group of miRNAs set forth in rows 1-20 of Table 1.

Another exemplary lethal RNAi molecule is a miRNA selected from the group of miRNAs set forth in Table 1 which is a miR-515 family member.

A further exemplary lethal RNAi molecule is an RNAi molecule selected from the group consisting of:
(i) a miRNA selected from the group consisting of miR-516b-5p, miR-517a-3p, miR-517c-3p, miR-518b, miR-519e-5p, and miR-526b-5p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii); (vii) a miRNA of any one of (i) to (iii) which has a chemical modification or a modified nucleobase; and
(viii) a nucleic acid encoding an RNAi molecule of any one of (i)-(v).

For example, the lethal RNAi molecule may be a miRNA selected from the group consisting of miR-516b-5p, miR-517a-3p, miR-517c-3p, miR-518b, miR-519e-5p, and miR-526b-5p.

In another example, the RNAi molecule is a synthetic lethal RNAi molecule. For example, the RNAi molecule may be a synthetic lethal RNAi molecule selected from the group consisting of:
(i) a miRNA selected from the group of miRNAs set forth in Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii); (vii) a miRNA of any one of (i) to (iii) which has a chemical modification or a modified nucleobase; and
(viii) a nucleic acid encoding an RNAi molecule of any one of (i)-(v).

For example, the RNAi molecule may be a synthetic lethal RNAi molecule selected from the group consisting of:
(i) a miRNA selected from the group of miRNAs set forth in rows 1-13 of Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii); (vii) a miRNA of any one of (i) to (iii) which has a chemical modification or a modified nucleobase; and
(viii) a nucleic acid encoding an RNAi molecule of any one of (i)-(v).

An exemplary synthetic lethal RNAi molecule is a miRNA selected from the group of miRNAs set forth in rows 1-13 of Table 2.

Another exemplary synthetic lethal RNAi molecule is a miRNA selected from the group of miRNAs set forth in Table 2 which is a miR-515 family member.

A further exemplary synthetic lethal RNAi molecule is an RNAi molecule selected from the group consisting of:
(i) a miRNA selected from the group consisting of miR-380-3p, miR-99b-5p and miR-485-3p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the synthetic lethal RNAi molecule may be a miRNA selected from the group consisting of miR-380-3p, miR-99b-5p and miR-485-3p.

In one example, administration of the RNAi molecule to the subject sensitizes a neuroblastoma cell in the subject to a chemotherapeutic agent.

In one example, administration of the RNAi molecule to the subject reduces a therapeutically effective dose of a chemotherapeutic agent which is effective for treating the neuroblastoma. Preferably, the therapeutically effective dose of the chemotherapeutic agent for treating neuroblastoma is reduced relative to a dose of the chemotherapeutic agent which is therapeutically effective in a subject who has not been, or will not be, administered the RNAi molecule. For example, the reduced dose of the chemotherapeutic agent may be a IC30 concentration of the chemotherapeutic agent. In another example, the reduced dose of the chemotherapeutic agent may be a IC10 concentration of the chemotherapeutic agent. In one example, an RNAi molecule which sensitizes a neuroblastoma cell to a chemotherapeutic agent is a synthetic lethal RNAi molecule as described herein e.g., as defined in Table 2, optionally wherein the synthetic lethal RNAi molecule is a synthetic lethal RNAi molecule as defined in rows 1-13 of Table 2. In one example, a synthetic lethal RNAi molecule which sensitizes a neuroblastoma cell to a chemotherapeutic agent is a miR-515 family member set forth in Table 2 in accordance with any example hereof. In one example, a synthetic lethal RNAi molecule which sensitizes a neuroblastoma cell to a chemotherapeutic agent is a synthetic lethal RNAi molecule corresponding to miR-380-3p, miR-99b-5p or miR-485-3p in accordance with any example hereof.

In one example, the nucleic acid encoding an RNAi molecule as described herein is comprised within an expression vector e.g., a plasmid or viral particle.

In another example, the nucleic acid encoding an RNAi molecule as described herein is comprised within an exosome or microvesicle.

In another example, the RNAi molecule or the nucleic acid encoding an RNAi molecule as described herein is comprised within an intact minicell.

In one example, the method described herein further comprises administering to the subject a chemotherapeutic agent. For example, the method may comprise administering to the subject a reduced therapeutically effective dose of a chemotherapeutic agent. A reduced therapeutically effective dose of the chemotherapeutic agent is one which is therapeutically effective in the treatment of neuroblastoma and reduced relative to a dose of the chemotherapeutic agent which is therapeutically effective in a subject who has not been, or will not be, administered the RNAi molecule. In one example, an RNAi molecule which may be administered in combination with a chemotherapeutic agent and which reduces a therapeutically effective dose of the chemotherapeutic agent is a synthetic lethal RNAi molecule as described herein e.g., as defined in Table 2, optionally wherein the synthetic lethal RNAi molecule is a synthetic lethal RNAi molecule as defined in rows 1-13 of Table 2. In one example, an RNAi molecule which may be administered in combination with a chemotherapeutic agent and which reduces a therapeutically effective dose of the chemotherapeutic agent is a miR-515 family member set forth in Table 2 in accordance with any example hereof. In one example, a synthetic lethal RNAi molecule which is administered in combination with a chemotherapeutic agent and which reduces a therapeutically effective dose of the chemotherapeutic agent is a synthetic lethal RNAi molecule corresponding to miR-380-3p, miR-99b-5p or mIR-485-3p in accordance with any example hereof.

In one example, the chemotherapeutic agent and the RNAi molecule are administered together. In one example, the chemotherapeutic agent and the RNAi molecule are administered concurrently. In one example, the chemotherapeutic agent and the RNAi molecule are administered sequentially, in any order.

In one example, the subject has previously received treatment with a chemotherapeutic agent.

In one example, the neuroblastoma is refractory to treatment with the chemotherapeutic agent in the absence of adjunctive treatment with the RNAi molecule. Alternatively, or in addition, the neuroblastoma is a recurrent neuroblastoma.

In accordance with an example in which a chemotherapeutic agent is, has or will be administered to the subject, the chemotherapeutic agent may be selected from a vinca alkaloid, a taxane, a platinum-based agent, an anthracycline, an alkylating agent, and/or a topoisomerase inhibitor. In one example, the chemotherapeutic agent is a vinca alkaloid e.g., vincristine, vinblastine, vinorelbine or vindesine. In one example, the chemotherapeutic agent is a taxane e.g., paclitaxel, docetaxel and cabazitaxel. In one example, the chemotherapeutic agent is a platinum-based agent e.g., cisplatin, carboplatin, oxaliplatin or nedaplatin. In one example, the chemotherapeutic agent is an anthracycline e.g., daunorubicin, doxorubicin, epirubicin, idarubicin or mitoxantrone. In one example, the chemotherapeutic agent is an alkylating agent e.g., cyclophosphamide, melphalan or dacarbazine. In one example, the chemotherapeutic agent is a topoisomerase inhibitor e.g., etoposide, a camptothecin, or a camptothecins derivative (such as topotecan). Other suitable chemotherapeutic drugs for treatment of neuroblastoma will be known in the art.

The present disclosure also provides for use of an RNA interference (RNAi) molecule in the preparation of a medicament for treating neuroblastoma in a subject suffering therefrom, wherein the RNAi molecule is selected from the group consisting of:
(i) a microRNA (miRNA) selected from the group of miRNAs set forth in Table 1 or Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

In one example, the disclosure provides for use of a lethal RNAi molecule in the preparation of the medicament. For example, the RNAi molecule may be a lethal RNAi molecule selected from the group consisting of:
(i) a miRNA selected from the group of miRNAs set forth in Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the lethal RNAi molecule may be selected from the group consisting of:
(i) a miRNA selected from the group of miRNAs set forth in rows 1-20 of Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

Exemplary lethal RNAi molecules useful in the preparation of the medicament are set forth in rows 1-20 of Table 1.

A further exemplary lethal RNAi molecule useful in the preparation of the medicament is a miRNA from the miR-515 family as set forth in Table 1.

A further exemplary lethal RNAi molecule for use in the preparation of a medicament of the disclosure is an RNAi molecule selected from the group consisting of:
(i) a miRNA selected from the group consisting of miR-516b-5p, miR-517a-3p, miR-517c-3p, miR-518b, miR-519e-5p, and miR-526b-5p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the lethal RNAi molecule may be a miRNA selected from the group consisting of miR-516b-5p, miR-517a-3p, miR-517c-3p, miR-518b, miR-519e-5p, and miR-526b-5p.

In another example, the disclosure provides for use of a synthetic lethal RNAi molecule in the preparation of the medicament. For example, the RNAi molecule may be a synthetic lethal RNAi molecule selected from the group consisting of:
(i) a miRNA selected from the group of miRNAs set forth in Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii); (vii) a miRNA of any one of (i) to (iii) which has a chemical modification or a modified nucleobase; and
(viii) a nucleic acid encoding an RNAi molecule of any one of (i)-(v).

For example, the synthetic lethal RNAi molecule may be selected from the group consisting of:
(i) a miRNA selected from the group of miRNAs set forth in rows 1-13 of Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

Exemplary synthetic lethal RNAi molecules useful in the preparation of the medicament are set forth in rows 1-13 of Table 2.

A further exemplary synthetic lethal RNAi molecule useful in the preparation of the medicament is a miRNA from the miR-515 family as set forth in Table 2. A further exemplary synthetic lethal RNAi molecule useful in the preparation of the medicament of the disclosure is an RNAi molecule selected from the group consisting of:
(i) a miRNA selected from the group consisting of miR-380-3p, miR-99b-5p and miR-485-3p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the synthetic lethal RNAi molecule may be a miRNA selected from the group consisting of miR-380-3p, miR-99b-5p and miR-485-3p.

In one example, the medicament may sensitize a neuroblastoma cell to a chemotherapeutic agent. Accordingly, treating neuroblastoma in the subject with the medicament may comprise sensitizing a neuroblastoma cell in the subject to a chemotherapeutic agent. The medicament which sensitizes a neuroblastoma cell to a chemotherapeutic agent may comprise a synthetic lethal RNAi molecule as described herein e.g., as defined in Table 2, optionally wherein the synthetic lethal RNAi molecule is a synthetic lethal RNAi molecule as defined in rows 1-13 of Table 2. In one example, a synthetic lethal RNAi molecule which sensitizes a neuroblastoma cell to a chemotherapeutic agent is a synthetic lethal RNAi molecule from the miR-515 family set forth in Table 2. In one example, a synthetic lethal RNAi molecule which sensitizes a neuroblastoma cell to a chemotherapeutic agent is a synthetic lethal RNAi molecule corresponding to miR-380-3p, miR-99b-5p or miR-485-3p in accordance with any example hereof.

Alternatively, or in addition, treating neuroblastoma in the subject with the medicament may comprise reducing a dose of a chemotherapeutic agent which is therapeutically effective for treating the neuroblastoma. For example, a medicament of the disclosure which reduces a therapeutically effective dose of a chemotherapeutic agent for treating neuroblastoma may comprise a synthetic lethal RNAi molecule as described herein e.g., as defined in Table 2, optionally wherein the synthetic lethal RNAi is a synthetic lethal RNAi molecule as defined in rows 1-13 of Table 2. In one example, a medicament of the disclosure which reduces a therapeutically effective dose of a chemotherapeutic agent for treating neuroblastoma may comprise a synthetic lethal RNAi molecule which is a synthetic lethal RNAi molecule from the miR-515 family set forth in Table 2. In one example, a synthetic lethal RNAi molecule which reduces a therapeutically effective dose of a chemotherapeutic agent for treating neuroblastoma is a synthetic lethal RNAi molecule corresponding to miR-380-3p, miR-99b-5p or mIR-485-3p in accordance with any example hereof.

In one example, the nucleic acid encoding an RNAi molecule as described herein is comprised in the medicament within an expression vector e.g., a plasmid or viral particle. In another example, the RNAi molecule is comprised in the medicament within an exosome or microvesicle. In another example, the RNAi molecule or the nucleic acid encoding an RNAi molecule is comprised in the medicament within a minicell e.g., an intact minicell.

In one example, the medicament further comprises a chemotherapeutic agent. For example, the medicament of the disclosure may comprise a reduced therapeutically effective dose of a chemotherapeutic agent. A reduced therapeutically effective dose of the chemotherapeutic agent is preferably one which is therapeutically effective in the treatment of neuroblastoma and reduced relative to a dose of the chemotherapeutic agent which is therapeutically effective in a subject who has not been, or will not be, administered the medicament comprising the RNAi molecule of the disclosure.

In another example, the medicament does not comprise a chemotherapeutic agent but is formulated for administration to the subject with a chemotherapeutic agent. In accordance with this example, the chemotherapeutic agent and the medicament comprising the RNAi molecule may be administered together. Alternatively, the chemotherapeutic agent and the medicament comprising the RNAi molecule may be administered concurrently. For example, the chemotherapeutic agent and the medicament comprising the RNAi molecule may be administered sequentially (in any order).

In one example, the medicament is for treating a subject suffering from neuroblastoma who has previously received treatment with a chemotherapeutic agent. For example, the neuroblastoma may be refractory to treatment with the chemotherapeutic agent e.g., in the absence of adjunctive treatment with the RNAi molecule. Alternatively, or in addition, the neuroblastoma may be a recurrent neuroblastoma.

In accordance with an example in which a chemotherapeutic agent is, has or will be administered to the subject as part of, or in conjunction with, the medicament of the disclosure, the chemotherapeutic agent may be selected from a vinca alkaloid, a taxane, a platinum-based agent, an anthracycline, an alkylating agent, and/or a topoisomerase inhibitor. In one example, the chemotherapeutic agent is a vinca alkaloid e.g., vincristine, vinblastine, vinorelbine or vindesine. In one example, the chemotherapeutic agent is a taxane e.g., paclitaxel, docetaxel and cabazitaxel. In one example, the chemotherapeutic agent is a platinum-based agent e.g., cisplatin, carboplatin, oxaliplatin or nedaplatin. In one example, the chemotherapeutic agent is an anthracycline e.g., daunorubicin, doxorubicin, epirubicin, idarubicin or mitoxantrone. In one example, the chemotherapeutic agent is an alkylating agent e.g., cyclophosphamide, melphalan or dacarbazine. In one example, the chemotherapeutic agent is a topoisomerase inhibitor e.g., etoposide, a camptothecin, or a camptothecins derivative (such as topotecan).

The present disclosure also provides an RNA interference (RNAi) molecule for use in treating neuroblastoma in a subject suffering therefrom, wherein the RNAi molecule is selected from the group consisting of:
(i) a microRNA (miRNA) selected from the group of miRNAs set forth in Table 1 or Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

In one example, the RNAi molecule for use in treating neuroblastoma is a lethal RNAi molecule as described herein. For example, the lethal RNAi molecule for use in treating neuroblastoma may be selected from the group consisting of:
(i) a miRNA selected from the group of miRNAs set forth in Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the lethal RNAi molecule for use in treating the neuroblastoma may be selected from the group consisting of:
(i) a miRNA selected from the group of miRNAs set forth in rows 1-20 of Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

Exemplary lethal RNAi molecules for use in treating neuroblastoma are set forth in rows 1-20 of Table 1.

A further exemplary lethal RNAi molecule for use in treating neuroblastoma is a miRNA from the miR-515 family which is selected from the group of miRNAs set forth in Table 1.

A further exemplary lethal RNAi molecule for use in treating neuroblastoma is an RNAi molecule selected from the group consisting of:
(i) a miRNA selected from the group consisting of miR-516b-5p, miR-517a-3p, miR-517c-3p, miR-518b, miR-519e-5p, and miR-526b-5p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the lethal RNAi molecule for use in treating neuroblastoma may be a miRNA selected from the group consisting of miR-516b-5p, miR-517a-3p, miR-517c-3p, miR-518b, miR-519e-5p, and miR-526b-5p.

In another example, the RNAi molecule for use in treating neuroblastoma is a synthetic lethal RNAi molecule as described herein. For example, the RNAi molecule may be a synthetic lethal RNAi molecule selected from the group consisting of: (i) a miRNA selected from the group of miRNAs set forth in Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the synthetic lethal RNAi molecule for use in treating neuroblastoma may be selected from the group consisting of:
(i) a miRNA selected from the group of miRNAs set forth in rows 1-13 of Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

Exemplary synthetic lethal RNAi molecules for use in treating neuroblastoma are set forth in rows 1-13 of Table 2.

A further exemplary synthetic lethal RNAi molecule for use in treating neuroblastoma is a miRNA from the miR-515 family which is selected from the group of miRNAs set forth in Table 2.

A further exemplary synthetic lethal RNAi molecule for use in treating neuroblastoma is an RNAi molecule selected from the group consisting of:
(i) a miRNA selected from the group consisting of miR-380-3p, miR-99b-5p and miR-485-3p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the synthetic lethal RNAi molecule may be a miRNA selected from the group consisting of miR-380-3p, miR-99b-5p and miR-485-3p.

In one example, treating the neuroblastoma comprises sensitizing a neuroblastoma cell to a chemotherapeutic agent. Accordingly, the RNAi molecule described herein may be for use in sensitizing a neuroblastoma cell to a chemotherapeutic agent as part of treatment. The RNAi molecule which sensitizes a neuroblastoma cell to a chemotherapeutic agent and which is for treating neuroblastoma may be a synthetic lethal RNAi molecule as described herein e.g., as defined in Table 2, optionally wherein the RNAi molecule is a synthetic lethal RNAi molecule as defined in rows 1-13 of Table 2. In one example, a synthetic lethal RNAi molecule which sensitizes a neuroblastoma cell to a chemotherapeutic agent and which is for treating neuroblastoma is a synthetic lethal RNAi molecule corresponding to miR-380-3p, miR-99b-5p or mIR-485-3p in accordance with any example hereof.

Alternatively, or in addition, treating neuroblastoma in the subject may comprise reducing a dose of a chemotherapeutic agent which is therapeutically effective for treating the neuroblastoma. For example, an RNAi molecule for use in treating neuroblastoma and which reduces a dose of a chemotherapeutic agent which is therapeutically effective in treatment is a synthetic lethal RNAi molecule as described herein e.g., as defined in Table 2, optionally wherein the RNAi molecule is a synthetic lethal RNAi molecule as defined in rows 1-13 of Table 2. In one example, an RNAi molecule for use in treating neuroblastoma and which reduces a dose of a chemotherapeutic agent which is therapeutically effective in treatment is a synthetic lethal RNAi molecule from the miR-515 family which is selected from the miRNAs set forth iin Table 2. In one example, the synthetic lethal RNAi molecule which reduces a dose of a chemotherapeutic agent which is therapeutically effective for treating neuroblastoma is a synthetic lethal RNAi molecule corresponding to miR-380-3p, miR-99b-5p or mIR-485-3p in accordance with any example hereof.

In one example, the nucleic acid encoding an RNAi molecule for use in treatment of neuroblastoma as described herein is comprised within an expression vector e.g., a plasmid or viral particle. In another example, the RNAi molecule for use in treatment of neuroblastoma as described herein is comprised within an exosome or microvesicle.

In one example, the RNAi molecule is for treating neuroblastoma in combination with a chemotherapeutic agent. For example, the RNAi molecule may be formulated together with the chemotherapeutic agent. Alternatively, the RNAi molecule is not formulated together with the chemotherapeutic agent, but is formulated such that it can be administered in combination with the chemotherapeutic agent for treatment of neuroblastoma. The RNAi molecule may be for administration together with the chemotherapeutic agent. Alternatively, the RNAi molecule may be for administration concurrent with administration of the chemotherapeutic agent. Alternatively, the RNAi molecule may be for administration with the chemotherapeutic agent sequentially, in any order.

In one example, the RNAi molecule described herein is for use in treating a subject suffering from neuroblastoma who has previously received treatment with a chemotherapeutic agent. For example, the RNAi molecule may be for use in treating neuroblastoma which is refractory to treatment with the chemotherapeutic agent e.g., in the absence of adjunctive treatment with the RNAi molecule. Alternatively, or in addition, the RNAi molecule may be for use in treating neuroblastoma which is recurrent.

In accordance with any example in which the RNAi molecule is for use in treating neuroblastoma in combination with a chemotherapeutic agent, the chemotherapeutic agent may be selected from a vinca alkaloid or an anthracycline. In one example, the chemotherapeutic agent is a vinca alkaloid e.g., vincristine, vinblastine, vinorelbine or vindesine. In one example, the chemotherapeutic agent is an anthracycline e.g., daunorubicin, doxorubicin, epirubicin, idarubicin or mitoxantrone.

The present disclosure also provides a pharmaceutical composition comprising one or more RNAi molecules, nucleic acids or expression vectors as described in any example hereof and a delivery vehicle suitable for delivery of the RNAi molecule to a neuroblastoma cell. For example, the delivery vehicle may be an intact minicell e.g., a bacterially derived intact minicell. Accordingly, the pharmaceutical composition may comprise an intact minicell e.g. bacterially derived intact minicell, containing one or more RNAi molecules of the disclosure or one or more expression vectors encoding same.

The one or more RNAi molecules comprised in the pharmaceutical composition may be selected from the group consisting of:
(i) a microRNA (miRNA) selected from the group of miRNAs set forth in Table 1 or Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

In one example, pharmaceutical composition comprises a lethal RNAi molecule described herein. For example, the lethal RNAi molecule may be selected from the group consisting of:
(i) a miRNA selected from the group of miRNAs set forth in Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the lethal RNAi molecule may be selected from the group consisting of:
(i) a miRNA selected from the group of miRNAs set forth in rows 1-20 of Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

Exemplary lethal RNAi molecules which may be formulated in the pharmaceutical composition are set forth in rows 1-20 of Table 1.

A further exemplary lethal RNAi molecule which may be formulated in the pharmaceutical composition of the disclosure is from the miR-515 family and is selected from the miRNAs set forth in Table 1.

A further exemplary lethal RNAi molecule which may be formulated in the pharmaceutical composition of the disclosure is an RNAi molecule selected from the group consisting of:
(i) a miRNA selected from the group consisting of miR-516b-5p, miR-517a-3p, miR-517c-3p, miR-518b, miR-519e-5p, and miR-526b-5p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the lethal RNAi molecule may be a miRNA selected from the group consisting of miR-516b-5p, miR-517a-3p, miR-517c-3p, miR-518b, miR-519e-5p, and miR-526b-5p.

In another example, the pharmaceutical composition comprises a synthetic lethal RNAi molecule described herein. For example, the synthetic lethal RNAi molecule may be selected from the group consisting of:
(i) a miRNA selected from the group of miRNAs set forth in Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the synthetic lethal RNAi molecule may be selected from the group consisting of:
(i) a miRNA selected from the group of miRNAs set forth in rows 1-13 of Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

Exemplary synthetic lethal RNAi molecules for inclusion in the pharmaceutical composition are set forth in rows 1-13 of Table 2.

Further exemplary synthetic lethal RNAi molecules for inclusion in the pharmaceutical composition of the disclosure are from the miR-515 family and selected from the miRNAs set forth in Table 2.

A further exemplary synthetic lethal RNAi molecule for inclusion in the pharmaceutical composition of the disclosure may be an RNAi molecule selected from the group consisting of:
(i) a miRNA selected from the group consisting of miR-380-3p, miR-99b-5p and miR-485-3p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the synthetic lethal RNAi molecule for inclusion in the pharmaceutical composition may be a miRNA selected from the group consisting of miR-380-3p, miR-99b-5p and miR-485-3p.

The pharmaceutical composition as described in any example hereof may further comprise a chemotherapeutic agent. In one example, the pharmaceutical composition of the disclosure may comprise a reduced therapeutically effective dose of a chemotherapeutic agent. A reduced therapeutically effective dose of the chemotherapeutic agent is one which is therapeutically effective in the treatment of neuroblastoma and reduced relative to a dose of the chemotherapeutic agent which is therapeutically effective in a subject who has not been, or will not be, administered the RNAi molecule of the disclosure. In one example, a pharmaceutical composition which has a reduced therapeutically effective dose of a chemotherapeutic agent comprises a synthetic lethal RNAi molecule as described herein e.g., as defined in Table 2, optionally wherein the synthetic lethal RNAi molecule is a synthetic lethal RNAi molecule as defined in rows 1-13 of Table 2. In one example, a synthetic lethal RNAi molecule which reduces a dose of a chemotherapeutic agent which is therapeutically effective for treating neuroblastoma is a synthetic lethal RNAi molecule from the miR-515 family which is selected from the miRNAs set forth in Table 2.

In one example, a synthetic lethal RNAi molecule which reduces a dose of a chemotherapeutic agent which is therapeutically effective for treating neuroblastoma is a synthetic lethal RNAi molecule corresponding to miR-380-3p, miR-99b-5p or mIR-485-3p in accordance with any example hereof.

In accordance with an example in which the pharmaceutical composition comprises a nucleic acid encoding an RNAi molecule as described herein, the nucleic acid is comprised within an expression vector e.g., a plasmid or viral particle.

In accordance with an example in which the pharmaceutical composition comprises a chemotherapeutic agent, the chemotherapeutic agent may be selected from a vinca alkaloid, a taxane, a platinum-based agent, an anthracycline, an alkylating agent, and/or a topoisomerase inhibitor. In one example, the chemotherapeutic agent is a vinca alkaloid e.g., vincristine, vinblastine, vinorelbine or vindesine. In one example, the chemotherapeutic agent is a taxane e.g., paclitaxel, docetaxel and cabazitaxel. In one example, the chemotherapeutic agent is a platinum-based agent e.g., cisplatin, carboplatin, oxaliplatin or nedaplatin. In one example, the chemotherapeutic agent is an anthracycline e.g., daunorubicin, doxorubicin, epirubicin, idarubicin or mitoxantrone. In one example, the chemotherapeutic agent is an alkylating agent e.g., cyclophosphamide, melphalan or dacarbazine. In one example, the chemotherapeutic agent is a topoisomerase inhibitor e.g., etoposide, a camptothecin, or a camptothecins derivative (such as topotecan).

The present disclosure also provides a method of preparing a pharmaceutical composition as described herein. For example, the present disclosure provides a method of preparing a pharmaceutical composition of the disclosure comprising co-incubating a plurality of intact minicells with one or more RNAi molecules, nucleic acids or expression vectors described herein in a buffer for a period sufficient to package the RNAi molecule, nucleic acid or expression vector into the minicell(s).

The one or more RNAi molecules may be selected from the group consisting of:
(i) a microRNA (miRNA) selected from the group of miRNAs set forth in Table 1 or Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

In one example, pharmaceutical composition is prepared with a lethal RNAi molecule described herein. For example, the lethal RNAi molecule may be selected from the group consisting of:
(i) a miRNA selected from the group of miRNAs set forth in Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the lethal RNAi molecule may be selected from the group consisting of:
(i) a miRNA selected from the group of miRNAs set forth in rows 1-20 of Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

Exemplary lethal RNAi molecules which may be formulated in the pharmaceutical composition are set forth in rows 1-20 of Table 1.

Exemplary lethal RNAi molecules which may be formulated in the pharmaceutical composition of the disclosure may be from the miR-515 family and selected from the miRNAs set forth in Table 1.

Exemplary lethal RNAi molecules which may be formulated in the pharmaceutical composition may also be selected from the group consisting of:
(i) a miRNA selected from the group consisting of miR-516b-5p, miR-517a-3p, miR-517c-3p, miR-518b, miR-519e-5p, and miR-526b-5p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the lethal RNAi molecule may be a miRNA selected from the group consisting of miR-516b-5p, miR-517a-3p, miR-517c-3p, miR-518b, miR-519e-5p, and miR-526b-5p.

In another example, the pharmaceutical composition is prepared with a synthetic lethal RNAi molecule described herein. For example, the synthetic lethal RNAi molecule may be selected from the group consisting of:
(i) a miRNA selected from the group of miRNAs set forth in Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the synthetic lethal RNAi molecule may be selected from the group consisting of:
(i) a miRNA selected from the group of miRNAs set forth in rows 1-13 of Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

Exemplary synthetic lethal RNAi molecules for formulation of a pharmaceutical composition are set forth in rows 1-13 of Table 2.

Exemplary synthetic lethal RNAi molecules for formulation of a pharmaceutical composition of the disclosure may be from the miR-515 family and selected from the miRNAs set forth in Table 2.

In one example, a pharmaceutical composition may also be prepared with a synthetic lethal RNAi molecule selected from the group consisting of:
(i) a miRNA selected from the group consisting of miR-380-3p, miR-99b-5p and miR-485-3p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the synthetic lethal RNAi molecule for use in formulating a pharmaceutical composition may be a miRNA selected from the group consisting of miR-380-3p, miR-99b-5p and miR-485-3p.

The pharmaceutical composition may be formulated with any one or more of the lethal and/or synthetic lethal RNAi molecules described herein.

Furthermore, the nucleic acid encoding the RNAi molecules described herein may be provided in the form of an expression vector e.g., a plasmid or viral particle.

In one example, the co-incubation step involves gentle shaking. In another example, the co-incubation step is static.

In one example, the co-incubation period is at least about half an hour, for example at least about one hour. However, any incubation time sufficient to achieve packing is contemplated.

In one example, the buffer comprises buffered saline, for example a IX phosphate buffer solution. The buffered saline can be in gelatin form.

In another example, the co-incubation is conducted at a temperature of about 4° C. to about 37° C.; about 20° C. to about 30° C.; about 25° C.; or about 37° C.

In other examples, the co-incubation can comprise about 10⁷, 10⁸, 10⁹, 10¹⁰, 10¹¹, 10¹² or 10¹³ minicells. It will be appreciated that the specific parameters of temperature, time, buffer, minicell concentration, etc. can be optimized for a particular combination of conditions.

The present disclosure also provides a kit comprising:
(a) a first container containing an RNA interference (RNAi) molecule selected from the group consisting of:
   (i) a microRNA (miRNA) selected from the group of miRNAs set forth in Table 1 or Table 2;
   (ii) a miRNA comprising an effector sequence of a miRNA at (i);
   (iii) a miRNA comprising an effector sequence which targets the same mRNA transcript sequence as an effector sequence of a miRNA at (i);
   (iv) a primary miRNA (pri-miRNA) corresponding to a miRNA of any one of (i) to (iii);
   (v) a precursor miRNA (pre-miRNA) corresponding to a miRNA any one of (i) to (iii);
   (vi) a miRNA mimic corresponding to a miRNA of any one of (i) to (iii);
   (vii) a miRNA of any one of (i) to (iii) which has a chemical modification or a modified nucleobase; and
   (viii) a nucleic acid encoding an RNAi molecule of any one of (i)-(v); and
(b) a second container containing a chemotherapeutic agent.

In one example, the RNAi molecule in the first container is a lethal RNAi molecule. For example, the RNAi molecule may be a lethal RNAi molecule selected from the group consisting of:
(i) a miRNA selected from the group of miRNAs set forth in Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the lethal RNAi molecule in the first container may be selected from the group consisting of:
(i) a miRNA selected from the group of miRNAs set forth in rows 1-20 of Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

Exemplary lethal RNAi molecules to be contained in the first container are set forth in rows 1-20 of Table 1.

Further exemplary lethal RNAi molecules to be contained in the first container are miRNAs from the miR-515 family and selected from the miRNAs set forth in Table 1.

Further exemplary lethal RNAi molecules to be contained in the first container may also be selected from the group consisting of:
(i) a miRNA selected from the group consisting of miR-516b-5p, miR-517a-3p, miR-517c-3p, miR-518b, miR-519e-5p, and miR-526b-5p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the lethal RNAi molecule to be contained in the first container may be a miRNA selected from the group consisting of miR-516b-5p, miR-517a-3p, miR-517c-3p, miR-518b, miR-519e-5p, and miR-526b-5p.

In another example, the first container of the kit comprises a synthetic lethal RNAi molecule of the disclosure. For example, the RNAi molecule may be a synthetic lethal RNAi molecule selected from the group consisting of:
(i) a miRNA selected from the group of miRNAs set forth in Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the synthetic lethal RNAi molecule of the first container of the kit of the disclosure may be selected from the group consisting of:
(i) a miRNA selected from the group of miRNAs set forth in rows 1-13 of Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

Exemplary synthetic lethal RNAi molecules for inclusion in the first container are set forth in rows 1-13 of Table 2.

Further exemplary synthetic lethal RNAi molecules to be contained in the first container are miRNAs from the miR-515 family and selected from the miRNAs set forth in Table 2.

A further exemplary synthetic lethal RNAi molecule for inclusion in the first container of a kit of the disclosure may be a synthetic lethal RNAi molecule selected from the group consisting of:
(i) a miRNA selected from the group consisting of miR-380-3p, miR-99b-5p and miR-485-3p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the synthetic lethal RNAi molecule for inclusion in the first container of the kit may be a miRNA selected from the group consisting of miR-380-3p, miR-99b-5p and miR-485-3p.

According to one example of the kit described herein, the nucleic acid encoding an RNAi molecule of the disclosure may be comprised within an expression vector e.g., a plasmid or viral particle. In another example, the RNAi molecule is comprised within an exosome or microvesicle.

In accordance with other aspects of the disclosure, it will be appreciated that the kit described herein is for treating neuroblastoma. Accordingly, the present disclosure contemplates use of the subject kit for treating neuroblastoma e.g., by administering to a subject the RNAi molecule of the first container and the chemotherapeutic agent of the second container. In one example, the chemotherapeutic agent and the RNAi molecule may be administered together. Alternatively, the chemotherapeutic agent and the RNAi molecule may be administered concurrently. For example, the chemotherapeutic agent and the RNAi molecule may be administered sequentially.

In one example, the kit is for treating a subject suffering from neuroblastoma who has previously received treatment with a chemotherapeutic agent. For example, the kit may be for treating neuroblastoma which is refractory to treatment with the chemotherapeutic agent by itself e.g., in the absence of adjunctive treatment with the RNAi molecule. Alternatively, or in addition, the neuroblastoma may be a recurrent neuroblastoma.

In one example, the second container of the kit comprises a reduced therapeutically effective dose of the chemotherapeutic agent. The reduced therapeutically effective dose of the chemotherapeutic agent is one which is therapeutically effective in the treatment of neuroblastoma and reduced relative to a dose of the chemotherapeutic agent which is therapeutically effective in a subject who has not been, or will not be, administered the RNAi molecule of the disclosure.

The chemotherapeutic agent comprised in the second container of the kit may be selected from a vinca alkaloid, a taxane, a platinum-based agent, an anthracycline, an alkylating agent, and/or a topoisomerase inhibitor. In one example, the chemotherapeutic agent is a vinca alkaloid e.g., vincristine, vinblastine, vinorelbine or vindesine. In one example, the chemotherapeutic agent is a taxane e.g., paclitaxel, docetaxel and cabazitaxel. In one example, the chemotherapeutic agent is a platinum-based agent e.g., cisplatin, carboplatin, oxaliplatin or nedaplatin. In one example, the chemotherapeutic agent is an anthracycline e.g., daunorubicin, doxorubicin, epirubicin, idarubicin or mitoxantrone. In one example, the chemotherapeutic agent is an alkylating agent e.g., cyclophosphamide, melphalan or dacarbazine. In one example, the chemotherapeutic agent is a topoisomerase inhibitor e.g., etoposide, a camptothecin, or a camptothecins derivative (such as topotecan). Other suitable chemotherapeutic drugs for treatment of neuroblastoma will be known in the art and are contemplated for use in the kit of the disclosure.

In one example, the RNAi molecule of the first container is provided in the form of a pharmaceutical composition as described in any example hereof.

The present disclosure also provides a method for treating neuroblastoma in a subject, comprising decreasing expression, and/or inhibiting a level of activity, of one or more of Rgs16, RbFox2 and/or Isl1 in the subject.

In one example, the method comprises administering to the subject an RNAi molecule which decreases expression, and/or inhibits a level of activity, of one or more of Rgs16, RbFox2 and/or Isl1 in the subject. Preferably, the RNAi molecule, when administered, increases neuroblastoma cell differentiation and/or inhibits or reduces neuroblastoma cell proliferation and/or inhibits or reduces neuroblastoma cell survival, in the subject.

In one example, the RNAi molecule is selected from the group consisting of a miRNA (or a corresponding pri-miRNA or pre-miRNA), an antisense nucleotide, a short interfering RNA (siRNA), and a short hairpin RNA (shRNA). An exemplary RNAi molecule in accordance with the present disclosure is a miRNA which targets a mRNA transcript of one or more of Rgs16, RbFox2 and/or Isl1, or a corresponding pri-miRNA or pre-miRNA which is processed into a miRNA which targets a mRNA transcript of one or more of Rgs16, RbFox2 and/or Isl1.

In accordance with any example of the method described herein, the administration of the RNAi molecule may comprise administering a nucleic acid encoding for the RNAi molecule and which is capable of being expressed in the subject. For example, the nucleic acid may be comprised within an expression vector e.g., a plasmid or viral particle, which is capable of expressing the RNAi molecule in the subject.

In another example, the nucleic acid encoding the RNAi molecule is comprised within an exosome or microvesicle.

In one example, the method comprises decreasing expression, or inhibiting a level of activity, of Rgs16 in the subject. Alternatively, or in addition, the method comprises decreasing expression, or inhibiting a level of activity, of RbFox2 in the subject. Alternatively, or in addition, the method comprises decreasing expression, or inhibiting a level of activity, of Isl1 in the subject.

The present disclosure also provides a composition for treating neuroblastoma according to the method described herein, wherein the composition comprises an RNAi molecule which decreases expression, or inhibits a level of activity, of one or more of Rgs16, RbFox2 and/or Isl1. The RNAi molecule in the composition is an RNAi molecule which decreases expression, or inhibits a level of activity, of one or more of Rgs16, RbFox2 and/or Isl1 as hereinbefore described. The composition may also comprise a pharmaceutically acceptable diluent or carrier.

The present disclosure also contemplates use of an RNAi molecule which decreases expression, or inhibits a level of activity, of one or more of Rgs16, RbFox2 and/or Isl1 in the preparation of a medicament for treating neuroblastoma in a subject suffering therefrom. The RNAi molecule in the medicament will be an RNAi molecule which decreases expression, or inhibits a level of activity, of one or more of Rgs16, RbFox2 and/or Isl1 as hereinbefore described. The medicament may further comprise a chemotherapeutic agent suitable for treatment of neuroblastoma as described herein.

As described herein, the RNAi molecule of the disclosure which decreases expression, or inhibits a level of activity, of one or more of Rgs16, RbFox2 and/or Isl1 may be used for treating a subject suffering from neuroblastoma. The subject suffering from neuroblastoma may have previously received treatment with a chemotherapeutic agent. For example, the neuroblastoma may be refractory to treatment with the chemotherapeutic agent e.g., in the absence of adjunctive treatment with the RNAi molecule. Alternatively, or in addition, the neuroblastoma may be a recurrent neuroblastoma.

### BRIEF DESCRIPTION OF FIGURES

FIG. 1 shows raw viability of (A) MDA-MB-231 cells, (B) MDA-MB-468 cells, (C) SK-BR-3 cells, (D) BT474 cells, (E) SHEP cells, (F) KELLY cells, (G) PC3 cells, (H) DU145 cells, normalised to a non-targeting control (OTP) or mock wells following transfection with miRNA mimics or inhibitors, using death genes or controls inducing varying levels of death to monitor transfection efficiency and to define dynamic range in each screen.

FIG. 2 provides an overview of the screen results in MDA-MB-231 breast cancer cell line. Y-axis displays CTG values normalised to the negative control (OTP) and the x-axis plots microRNA libraries (mimics or inhibitors). Controls are colour-coded and they include PLK1, KIF11, TOX (positive controls), and mock, OTP, noncoding inhibitor control, noncoding mimic control (negative controls).

FIG. 3 provides an overview of the screen results in (A) MDA-MB-231 cells, (B) MDA-MB-468 cells, (C) SK-BR-3 cells, (D) BT474 cells, (E) SHEP cells, (F) KELLY cells, (G) PC3 cells, (H) DU145 cells. In each case, the Y-axis displays CTG values normalised to the negative control (OTP) and the x-axis plots microRNA libraries (mimics or inhibitors). Controls are colour-coded and they include PLK1, KIF11, TOX (positive controls), and mock, OTP, noncoding inhibitor control, noncoding mimic control (negative controls).

FIG. 4 illustrates expression levels of endogenous microRNAs in breast cancer cell lines screened.

FIG. 5 illustrates the experimental designed used in the genome-wide miRNA functional screens.

FIG. 6 illustrates the experimental time line for genome-wide miRNA functional screens.

FIG. 7 provides results of the genome-wide miRNA functional screen. Cell viability following overexpression of each miRNA as single-agent is displayed on the x-axis. Cell viability following overexpression of each miRNA in combination with low dose IC30 is displayed on the Y-axis (normalised to treatment matched scrambled controls).

FIG. 8 provides representative results from miRNA validation screens in (A) KELLY and (B) SHEP neuroblastoma cell lines. The respective graphs plot normalised viability values for selected candidate lethal microRNA mimics identified in the in the primary screen against their values in the secondary validation screen. The screens were performed in different facilities and on different transfection platforms.

FIG. 9 is a graph plotting normalised viability values for candidate synthetic lethal microRNA mimics identified in the primary screen against their values in the secondary validation screen in Kelly cells.

FIG. 10 is a flow diagram illustrating the structure of the microRNA functional database and search/prediction tool (a) Mining of the screening data gives a list of microRNAs with a lethal effect in one or more cancer cell lines. (b) There is an option of excluding microRNAs that affect cardiomyocytes and normal breast cancer cells. (c) The microRNAs are further subjected to 5 different target prediction algorithms and the targets are ranked according to the strength of predictions and the number of microRNAs that target them. The targets are prioritised based on the target expression levels in the relevant cell line as well as their impact on the survival of the same cell line (siRNA screens). (d) The final list of targets can be further analysed using internal and external gene enrichment tools.

FIG. 11 shows output from the prediction tool with database metrics.

FIG. 12 identifies genes predicted to be regulated by the miRNAs identified in the functional genomic screen performed in Example 1 on the basis that they killed over 80% of KELLY cells compared to non-targeting control. Each of the 13 miRNAs for which the target prediction was performed was previously known to regulate MYCN.

FIG. 13 illustrates changes in mRNA levels of (A) MYCN and (B) LIN28B KELLY, cells transfected with let-7b-5p, miR-101-3, miR-202-3p, or scrambled (OTP-NT), as assessed using qPCR. B2M was used as a housekeeping gene. The respective graphs plot normalised fold change of mRNA levels compared with scrambled control (n=3, *p-value ≤0.05).

FIG. 14 shows novel factors predicted as being important to neuroblastoma development as identified using the prediction tool (as described in Example 5).

FIG. 15 illustrates the level of change in expression of (A) HAND1, (B) RBFOX2, (C) RGS16 and (D) ACVR2B in KELLY cells transfected with let-7b-5p, miR-101-3, miR-202-3p, or scrambled (OTP-NT), as determined by qPCR. B2M was used as a housekeeping gene. The graphs plot normalised fold change of mRNA levels compared with scrambled control (n=3, *p-value ≤0.05).

FIG. 16 illustrates the inhibitory activity of let-7b-5p in a Luciferase reporter assay. The level of knockdown of (A) HAND1 and (B) RGS 16 was determined in HEK293T cells co-transfected with NT or let-7b-5p, and HAND1 or RGS16 reporter plasmid encoding either WT or mutated 3′UTR. 24 hr following trabsfection, cell lysates were prepared and subjected to dual luciferase assay to measure both firefly (control) and renilla luciferase (experimental signal). The graph plots fold change relative to NT control and is representative of 3 independent experiments. The significance was determined using two-way ANOVA with Sidak's multiple comparisons test (n=3, *p-value ≤0.05).

FIG. 17 shows the level of (A) cell confluence and (B) cell viability of KELLY cells following transfection with siRNAs against MYCN, LIN28B, HAND1, RGS16, ACVR2B, RBFOX2, or scrambled (OTP-NT) in a 384-well format. Both cell confluence and cell viability were plotted as values normalised to the scrambled control (OTP-NT). The graph is an average of 3 independent experiments (*p-value ≤0.05).

FIG. 18 shows that Isl1, a LIM homeobox transcription factor, is highly expressed in primary neuroblastoma tumours and cell lines, but not other tumour types.

FIG. 19 shows the level of change in expression of ISL1 in KELLY cells following transfection with miR-517a-3p or scrambled control (OTP-NT), as assessed by qPCR. B2M was used as a housekeeping gene. The graph plots normalised fold change of mRNA levels compared with scrambled control (n=3, *p-value ≤0.05).

FIG. 20 illustrates the inhibitory activity of miR-517a in a Luciferase reporter assay. The level of knockdown of ISL1 was determined in HEK293T cells co-transfected with NT or let-7b-5p, and ISL1 reporter plasmid encoding either WT or mutated ISL1 3′UTR. 24 hr following trabsfection, cell lysates were prepared and subjected to dual luciferase assay to measure both firefly (control) and renilla luciferase (experimental signal). The graph plots fold change relative to NT control and is representative of 3 independent experiments. The significance was determined using two-way ANOVA with Sidak's multiple comparisons test (n=3, *p-value ≤0.05).

FIG. 21 shows confluence of KELLY cells following transfection with a siRNA against ISL1 or scrambled control (OTP-NT), plotted as values normalised to the scrambled control. The graph is an average of 3 independent experiments (*p-value ≤0.05).

FIG. 22 illustrates that confluence and morphology of KELLY cells is changed following transfection with a siRNA against ISL1 or scrambled control (OTP-NT), suggesting changes in differentiation.

FIG. 23 shows cell cycle profiles for KELLY cells 96 hours after transfection with a siRNA against ISL1 or a scrambled control (OTP-NT). The graphs are representative of 3 independent experiments.

FIG. 24 shows the level of change in expression of differentiation markers GAP43, NEFL, and NTRK1 in KELLY cells following transfection with a siRNA against ISL1 or scrambled control (OTP-NT), as determined by qPCR. B2M and GAPDH were used as housekeeping genes. The graph plots normalised fold change of mRNA levels compared with scrambled control (n=3, p-value ≤0.05).

FIG. 25 provides Kaplan-Meier curves of event-free survival for 3 synthetic lethal miRNAs (A-C) and 5 lethal miRNAs (D-H), generated from an unpublished cohort of about 200 NB patients. Yellow line represents the quartile of patients with lowest expression of the given miRNA.

### DETAILED DESCRIPTION

### General

Throughout this specification, unless specifically stated otherwise or the context requires otherwise, reference to a single step, feature, composition of matter, group of steps or group of features or compositions of matter shall be taken to encompass one and a plurality (i.e. one or more) of those steps, features, compositions of matter, groups of steps or groups of features or compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant DNA, recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

Throughout this specification, unless the context requires otherwise, the word “comprise”, or variations such as “comprises” or “comprising”, is understood to imply the inclusion of a stated step or element or integer or group of steps or elements or integers but not the exclusion of any other step or element or integer or group of elements or integers.

The term “and/or”, e.g., “X and/or Y” shall be understood to mean either “X and Y” or “X or Y” and shall be taken to provide explicit support for both meanings or for either meaning.

The term “about” as used herein refers to a range of +/−10% of the specified value.

### Selected Definitions

By “RNA” is meant a molecule comprising at least one ribonucleotide residue. By “ribonucleotide” is meant a nucleotide with a hydroxyl group at the 2′ position of a β-D-ribo-furanose moiety. The terms include double-stranded RNA, single-stranded RNA, isolated RNA such as partially purified RNA, essentially pure RNA, synthetic RNA, recombinantly-produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant disclosure can also comprise non-standard nucleotides, such as non-naturally occurring nucleotides or chemically synthesized nucleotides or deoxynucleotides. These altered RNAs can be referred to as analogs or analogs of naturally-occurring RNA. In one example, all of the residues in the RNAi molecule are ribonucleotides.

The term “RNA interference” or “RNAi” refers generally to RNA-dependent silencing of gene expression initiated by double stranded RNA (dsRNA) molecules in a cell's cytoplasm. The dsRNA molecule reduces or inhibits transcription products of a target nucleic acid sequence, thereby silencing the gene or reducing expression of that gene. Thus, the term “RNA interference molecule” or “RNAi molecule” refers to a molecule that is capable of eliciting “RNA interference” or “RNAi”. In some examples, this term refers to a miRNA or a precursor thereof e.g., a pri-miRNA or pre-miRNA or a miRNA mimic, and in other examples this term is used to refer to a nucleic acid which encodes a miRNA or a precursor thereof of the disclosure.

As used herein, the term “microRNA”, “miRNA”, “miR” or similar refers to a class of small non-coding RNA molecules of between about 18 and about 25 nucleobases in length which comprise a sequence capable of hybridising to sequences within target messenger RNA transcripts (mRNAs), and which function in RNA silencing and post-transcriptional regulation of gene expression. A mature miRNA molecule comprises an “effector sequence” which binds to the a target miRNA sequence. When in a double stranded form, a miRNA also comprises a sequence which is complementary to and duplexed with the effector sequence through hydrogen bonding which is referred to herein as the “effector complement sequence”. Each of the effector and effector complement sequences will be of similar length, for example, about 18 to 25 nucleotides in length such as 19 to 23 nucleotides. The effector sequence of the miRNA is the sequence loaded into the RISC complex and which binds to the target mRNA. Target sequences for miRNA are typically found within mRNA 3′- and 5′ untranslated regions (UTRs) as well as within mRNA coding regions. Some miRNAs target single mRNAs at multiple sites. miRNA seeds regions are predicted to target on the order of 200 genes each, and most mRNA are targeted by multiple miRNA. Upon binding of RISC-loaded miRNAs to one or more target mRNA(s), the target mRNA(s) become subject to translational arrest, mRNA degradation, or mRNA de-adenylation. In addition to the regulatory effects of miRNA, paradigms of post-transcriptional regulation of pri-miRNA-maturation are emerging from current studies that reveal the tight regulation of miRNA maturation. miRNAs which are particularly useful for treating neuroblastoma are described herein e.g., in Tables 1 and 2.

It will be appreciated that miRNAs of the disclosure can be obtained from a precursor molecule (referred to herein as a “precursor miRNA”, “pre-miRNA”, “pre-miR” or “iRNA precursor”) through natural processing routes (e.g., using intact cells or cell lysates) or by synthetic processing routes (e.g., using isolated processing enzymes, such as isolated Dicer, Argonaut, or RNAase III). It will be appreciated that the miRNA molecule can also be produced directly by biological or chemical syntheses, without having been processed from a pre-miRNA. Accordingly, the terms “microRNA”, “miRNA” and “miR” encompasses both miRNAs produced through pre-miRNA processing by Dicer and miRNAs produced through direct biological or chemical synthesis. A number of studies have looked at the base-pairing requirement between miRNA and its mRNA target for achieving efficient inhibition of translation (reviewed by Bartel (2004) Cell 116:281). In mammalian cells, the first 8 nucleotides of the miRNA may be important. However, other parts of the microRNA may also participate in miRNA binding. Moreover, sufficient base pairing at the 3′ can compensate for insufficient pairing at the 5′ (Brennecke et al., (2005) PLoS 3-e85). Computation studies, analyzing miRNA binding on whole genomes have suggested a specific role for bases 2-7 at the 5′ of the miRNA in target binding but the role of the first nucleotide, found usually to be “A” has also been recognized (Lewis et al. (2005) Cell, 120:15). Similarly, nucleotides 1-7 or 2-8 were used to identify and validate targets by Krek et al., (2005), Nat Genet 37:495).

As used herein, the term “precursor miRNA”, “pre-miRNA,” or “pre-miR”, “miR precursor” or similar means a non-coding RNA having a hairpin structure, which contains a miRNA of the disclosure. For example, a miRNA may be present in one arm of the hairpin precursor which lacks large internal loops or bulges. In certain examples, a pre-miRNA is the product of cleavage of a primary mi-RNA transcript, (also referred to herein as a “primary miRNA”, “pri-miRNA” or “pri-miR”) by the double-stranded RNA-specific ribonuclease known as Drosha. However, in other examples, pre-miRNAs can also be produced directly by biological or chemical synthesis without having been processed from a pri-miR. The pre-miRNA sequence may comprise from 45-90, 60-80 or 60-70 nucleotides. A pre-miRNA will comprise a stem or double-stranded region comprising the miRNA duplex (i.e., a region of the nucleic acid molecule that is in a double stranded conformation via hydrogen bonding between the nucleotides), and a loop region of unpaired nucleotides at the terminal end of the stem. The unpaired nucleotides of the loop region of a pre-miRNA are also referred to herein as a “stem loop”. The duplexed region of the pre-miRNA will include the effector sequence of the miRNA (that binds to a target mRNA) hydrogen bonded to its cognate effector complement sequence A pre-miRNA molecule described herein may comprise more than one miRNA of the disclosure, including a miRNA as described herein and one or more variants thereof. The sequence of the pre-miRNA may also be that of the corresponding pri-miRNA molecule excluding from 0-160 nucleotides from the 5′ and 3′ ends of the pri- miRNA.

As used herein, the term “pri-miRNA” means a primary miRNA transcript that is cleaved by Drosha or an equivalent protein. The pri-miRNA sequence may comprise from 45-250, 55-200, 70-150 or 80-100 nucleotides. As described above with reference to a pre-miRNA, the pri-miRNA may be a hairpin structure formed between a first and a second nucleic acid sequence which are substantially complementary to one another. One of the first and second nucleic acid sequences will comprise the effector sequence of the mature miRNA and the other will comprise the cognate effector complement sequence. The first and second nucleic acid sequence may be from 37-50 nucleotides. The first and second nucleic acid sequence may be separated by a third sequence of from 4-20, 8-12 or 10 nucleotides which forms a term loop (also referred to as a stem loop). The hairpin structure may have a free energy less than −25 Kcal/mole as calculated by the Vienna algorithm with default parameters, as described in Hofacker et al., Monatshefte f. Chemie 125: 167-188 (1994), the contents of which are incorporated herein.

As used herein, the term “miRNA mimic” refers to synthetic small non-coding RNAs capable of entering the RNAi pathway and regulating gene expression. As used herein, the term “synthetic miRNA” refers to any type of miRNA sequence, other than an endogenous miRNA. miRNA mimics imitate the function of endogenous miRNAs and can be designed as mature, double stranded (duplex) molecules or mimic precursors e.g., pri-miRNAs or pre-miRNAs. The miRNA mimic may comprise an effector sequence which is substantially identical to the effector sequence of the corresponding endogenous miRNA. miRNA mimics can be comprised of modified and/or unmodified RNA, DNA, RNA-DNA hybrids or alternative nucleic acid chemistries.

As used herein, the term “effector sequence” (also referred to as a “guide strand”) is a sequence of about 18 to 25 nucleobases within the miRNA, pre-miRNA or pri-miRNA of the disclosure which is substantially complementary to a target mRNA sequence, which in the present case is associated with neuroblastoma.

The term “effector complement sequence” as used herein refers to the sequence within a miRNA duplex, pre-miRNA or pri-miRNA of the disclosure which is of similar length and of sufficient complementarity to the effector sequence such that it can anneal to the effector sequence to form a duplex. In this regard, the effector complement sequence will be substantially homologous to a region of the target mRNA transcript. As will be apparent to the skilled person, the term “effector complement sequence” can also be referred to as the “complement of the effector sequence” or “passenger strand”.

As used herein, the term “duplex”, “duplex region” or “double stranded” refers to a region within two complementary or substantially complementary RNA sequences, or in two complementary or substantially complementary regions of a single-stranded RNA, that form base pairs with one another, either by Watson-Crick base pairing or any other manner that allows for a stabilized duplex between the nucleotide sequences that are complementary or substantially complementary. It will be understood by the skilled person that within a duplex region, 100% complementarity is not required; substantial complementarity is allowable. Substantial complementarity may include 75% or greater complementarity. For example, a single mismatch in a duplex region consisting of 19 base pairs (i.e., 18 base pairs and one mismatch) results in 94.7% complementarity, rendering the duplex region substantially complementary. In another example, two mismatches in a duplex region consisting of 19 base pairs (i.e., 17 base pairs and two mismatches) results in 89.5% complementarity, rendering the duplex region substantially complementary. In yet another example, three mismatches in a duplex region consisting of 19 base pairs (i.e., 16 base pairs and three mismatches) results in 84.2% complementarity, rendering the duplex region substantially complementary, and so on. However, where mismatches are present, it is preferred that they are not located within the region corresponding to the seed region of the miRNA i.e., nucleotides 2-8.

A pri-miRNA or a pre-miRNA as described herein may be provided as a hairpin or stem loop structure, with a duplex region comprised of at least the effector sequence and effector complement sequence of a corresponding miRNA linked by at least between at least 2 nucleotide e.g., between 8 and 14 nucleotides, which is termed a “stem loop”.

As used herein, the term “complementary” with regard to a sequence refers to a complement of the sequence by Watson-Crick base pairing, whereby guanine (G) pairs with cytosine (C), and adenine (A) pairs with either uracil (U) or thymine (T). A sequence may be complementary to the entire length of another sequence, or it may be complementary to a specified portion or length of another sequence. One of skill in the art will recognize that U may be present in RNA, and that T may be present in DNA. Therefore, an A within either of an RNA or DNA sequence may pair with a U in an RNA sequence or T in a DNA sequence.

As used herein, the term “substantially complementary” or “substantial complementarity” is used to indicate a sufficient degree of complementarity or precise pairing such that stable and specific binding occurs between nucleic acid sequences e.g., between the effector sequence and the effector complement sequence or between the effector sequence and the target sequence. It is understood that the sequence of a nucleic acid need not be 100% complementary to that of its target or complement. The term encompasses a sequence complementary to another sequence with the exception of an overhang. In some cases, the sequence is complementary to the other sequence with the exception of 1-2 mismatches. In some cases, the sequences are complementary except for 1 mismatch. In some cases, the sequences are complementary except for 2 mismatches. In other cases, the sequences are complementary except for 3 mismatches. In some cases, the sequences are complementary except for 4 mismatches. In some cases, the sequences are complementary except for 5 mismatches. In yet other cases, the sequences are complementary except for 6 mismatches, and so on. However, as discussed herein, where mismatches are present, it is preferred that they are not located within the region corresponding to the seed region of the miRNA i.e., nucleotides 2-8.

Reference herein to a “lethal” RNAi molecule refers to an RNAi molecule which is lethal to a neuroblastoma cell when administered alone as a therapeutic agent.

Reference herein to a “synthetic lethal” RNAi molecule refers to an RNAi molecule which confers a lethal phenotype to a neuroblastoma cell when administered in combination with low dose IC30 chemotherapy. For example, the dose of the chemotherapeutic agent would otherwise be insufficient to confer the lethal phenotype to the neuroblastoma cell in the absence of the “synthetic lethal” RNAi molecule of the disclosure. In another example, the neuroblastoma may be refractory or non-responsive to treatment with the chemotherapeutic agent in the absence of the “synthetic lethal” RNAi molecule of the disclosure.

As used herein, “a nucleic acid encoding an RNAi molecule” or similar refers to a nucleic acid comprising a DNA sequence which serves as a template for transcription of the respective RNAi molecule(s) of the disclosure. The term “encoded”, “encodes” or encoding”, as used in the context of an RNAi molecule of the disclosure, shall be understood to mean an RNA is capable of being transcribed from a DNA template. Accordingly, a nucleic acid that encodes a miRNA, pre-miRNA or pri-miRNA of the disclosure will comprise a DNA sequence which serves as a template for transcription of the respective RNAi molecule.

A “vector” will be understood to mean a vehicle for introducing a nucleic acid into a cell. Vectors include, but are not limited to, plasmids, phagemids, viruses, bacteria, and vehicles derived from viral or bacterial sources. A “plasmid” is a circular, double-stranded DNA molecule. A useful type of vector for use in accordance with the present disclosure is a viral vector, wherein heterologous DNA sequences are inserted into a viral genome that can be modified to delete one or more viral genes or parts thereof. Certain vectors are capable of autonomous replication in a host cell (e.g., vectors having an origin of replication that functions in the host cell). Other vectors can be stably integrated into the genome of a host cell, and are thereby replicated along with the host genome. As used herein, the term “expression vector” will be understood to mean a vector capable of expressing an RNAi molecule of the disclosure.

A vector for expressing an RNAi molecule of the disclosure will comprise a suitable expression construct (referred herein as a “RNAi expression construct”) comprising a DNA template from which the RNAi molecule can be transcribed. A “RNAi expression construct” will be a nucleic acid comprising a DNA sequence which, when transcribed, produces an RNAi molecule of the disclosure. In one example, an RNAi expression construct described herein may comprise a nucleic acid which is transcribed as a single RNA that is capable of self-annealing into a hairpin structure with a duplex region linked by at least 2 nucleotides e.g., a pre-miRNA or a pri-miRNA, to produce a single miRNA. In another example, an RNAi expression construct described herein may comprise a nucleic acid which is transcribed as a single RNA that is capable of self-annealing into a hairpin structure with a duplex region linked by at least 2 nucleotides e.g., a pre-miRNA or a pri-miRNA, to produce multiple miRNAs. In another example, an RNAi expression construct described herein may comprise a nucleic acid which is transcribed as multiple RNA transcripts each capable of self-annealing into a hairpin structure with a duplex region linked by at least 2 nucleotides e.g., a pre-miRNA or a pri-miRNA, and which can each be processed into one or more miRNAs of the disclosure. The RNAi expression construct described herein may be operable-linked to a promoter within the expression vector.

As used herein, the term “operably-linked” or “operable linkage” (or similar) means that the nucleic acid sequence coding for the RNAi molecule is linked to, or in association with, a regulatory sequence, e.g., a promoter, in a manner which facilitates expression of the RNAi molecule. Regulatory sequences include promoters, enhancers, and other expression control elements that are art-recognized and are selected to direct expression of the coding sequence.

The term “minicell”, as used herein, refers to anucleate forms of bacterial cells, engendered by a disturbance in the coordination, during binary fission, of cell division with DNA segregation. Minicells are distinct from other small vesicles that are generated and released spontaneously in certain situations and, in contrast to minicells, are not due to specific genetic rearrangements or episomal gene expression. In the context of the present disclosure, the minicells are intact since other “denuded” forms, such as spheroplasts, poroplasts, protoplasts, would leak the packaged functional nucleic acid and would not be therapeutically effective. The intact minicell membrane allows the payload to be retained within the minicell and is released intracellular\hat{ } within the target host mammalian cell. Accordingly, it is preferred that the minicells used in the present disclosure have intact cell walls (“intact minicells”).

The term “neuroblastoma” refers to malignant tumor of an undifferentiated cell that differentiates to a neural cell (neuroblast). Neuroblastoma is a childhood cancer of the sympathetic nervous system, which accounts for approximately 15% of all cancer deaths in children. The methods and reagents of the present disclosure are contemplated for use in treatment of all forms of neuroblastoma, including those which are refractory to treatment with chemotherapeutic agents and those that are recurrent.

As used herein, the term “refractory” or “refractory to treatment” in the context of neuroblastoma is intended to mean neuroblastoma which is partially or wholly non-responsive or resistant to treatment with a therapeutic agent for treating neuroblastoma e.g., such as a chemotherapeutic agent. A neuroblastoma which is refractory to a treatment may become refractory after a period of successful treatment, or may be non-responsive to treatment from the outset of treatment.

A “recurrent neuroblastoma” or “relapsed neuroblastoma” as referred to herein is a neuroblastoma which has returned in a patient who has already undergone effective initial treatment for the disease and achieved remission. Approximately half of children who are treated for high-risk neuroblastoma and achieve an initial remission will suffer from recurrent neuroblastoma.

The term “sensitize” or the phrase “sensitizing a neuroblastoma” to a chemotherapeutic agent means that the RNAi molecule of the disclosure alters neuroblastoma cells in a way that allows for more effective treatment of the neuroblastoma with the corresponding chemotherapeutic agent. For example, providing a cell or subject with an effective amount of an RNAi molecule of the disclosure may sensitize a neuroblatoma cell, which would otherwise be resistant or less-responsive, to treatment with a chemotherapeutic agent.

As used herein, the terms “treating”, “treat” or “treatment” and variations thereof, refer to clinical intervention designed to alter the natural course of the individual or cell being treated during the course of clinical pathology. Desirable effects of treatment include decreasing the rate of disease progression, ameliorating or palliating the disease state, and remission or improved prognosis. It follows that treatment of neuroblastoma includes inhibiting or reducing expression of one or more genes associated with neuroblastoma, sensitizing a neuroblastoma to a chemotherapeutic agent, reducing a therapeutically-effective dose of a chemotherapeutic agent for treating neuroblastoma, improving efficacy of a chemotherapeutic agent to treat neuroblastoma, increasing survival time of a subject suffering from neuroblastoma, reducing size of a neuroblastoma tumour, reducing expression of one or more cancer biomarkers associated with neuroblastoma, reducing or slowing rate of growth of a neuroblastoma tumour, preventing, inhibiting or slowing spread of cancer from the primary site to other organs or tissue (metastasis), and/or reducing severity of symptoms associated with neuroblastoma. An individual is successfully “treated”, for example, if one or more of the above treatment outcomes is achieved.

A “therapeutically effective amount” is at least the minimum concentration or amount required to effect a measurable improvement of a particular disease (e.g., neuroblastoma). A therapeutically effective amount herein may vary according to factors such as the disease state, age, sex, and weight of the patient, and the ability of the RNAi molecule to elicit a desired response in the individual. A therapeutically effective amount is also one in which any toxic or detrimental effects of the RNAi molecule are outweighed by the therapeutically beneficial effects.

As used herein, the “subject” or “patient” can be a human or non-human animal suffering from neuroblastoma. The “non-human animal” may be a primate, livestock (e.g. sheep, horses, cattle, pigs, donkeys), companion animal (e.g. pets such as dogs and cats), laboratory test animal (e.g. mice, rabbits, rats, guinea pigs), performance animal (e.g. racehorses, camels, greyhounds) or captive wild animal. In one example, the subject or patient is a mammal. In one example, the subject or patient is a primate. In one example, the subject or patient is human. In any example of the methods disclosed herein, the methods may further comprise a step of selecting a patient suitable for treatment with the treatment method disclosed herein. The step of selecting may comprise identifying the patient as one who is suffering from, or who has suffered from neuroblastoma.

The terms “reduced expression”, “reduction in expression” or similar, refer to the absence or an observable decrease in the level of protein and/or mRNA product from the target gene. The decrease does not have to be absolute, but may be a partial decrease sufficient for there to a detectable or observable change as a result of the RNAi effected by the miRNA of the disclosure. The decrease can be measured by determining a decrease in the level of mRNA and/or protein product from a target nucleic acid relative to a cell lacking the RNAi molecule, and may be as little as 1%, 5% or 10%, or may be absolute i.e., 100% inhibition. The effects of the decrease may be determined by examination of the outward properties i.e., quantitative and/or qualitative phenotype of the cell or organism, and may also include detection and/or quantitation of one or more markers of neuroblastoma following administration of an RNAi molecule of the disclosure.

### RNAi Molecules

The present disclosure contemplates the use of certain RNAi molecules in the treatment of neuroblastoma. In this regard, the present inventors performed functional screens with miRNA libraries in neuroblastoma cell lines to determine the effect of miRNAs on cellular viability of neuroblastoma cells. In so doing, the inventors have identified miRNAs with a previously unrecognised role in survival of neuroblastoma cells. In particular, the inventors have identified miRNAs which, when overexpressed or administered as a stand-alone treatment, are lethal to neuroblastoma cells (‘lethal’ miRNAs). The inventors have also identified that certain miRNAs screened resulted in lethal phenotype to neuroblastoma cells when administered in combination with low dose IC30 chemotherapy (‘synthetic lethal’ miRNAs).

RNAi molecules useful in treatment of neuroblastoma preferably include the seed sequence of a miRNA selected from the group of miRNAs set forth in Table 1 or Table 2. Exemplary RNAi molecules which are useful in the treatment of neuroblastoma may be selected from the group consisting of:
(i) a microRNA (miRNA) selected from the group of miRNAs set forth in Table 1 or Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

TABLE 1
Lethal miRNAs in neuroblastoma cells

| | | | SEQ ID |
|---|---|---|---|
| Row | mature miRNA | mature miRNA sequence | NO |
| 1 | hsa-miR-876-3p | UGGUGGUUUACAAAGUAAUUCA | 1 |
| 2 | hsa-miR-101-3p | UACAGUACUGUGAUAACUGAA | 2 |
| 3 | hsa-miR-550a-3p | UGUCUUACUCCCUCAGGCACAU | 3 |
| 4 | hsa-miR-634 | AACCAGCACCCCAACUUUGGAC | 4 |
| 5 | hsa-miR-542-3p | UGUGACAGAUUGAUAACUGAAA | 5 |
| 6 | hsa-miR-103a-2-5p | AGCUUCUUUACAGUGCUGCCUUG | 6 |
| 7 | hsa-miR-654-5p | UGGUGGGCCGCAGAACAUGUGC | 7 |
| 8 | hsa-miR-1289 | UGGAGUCCAGGAAUCUGCAUUUU | 8 |
| 9 | hsa-miR-544a | AUUCUGCAUUUUUAGCAAGUUC | 9 |
| 10 | hsa-miR-892b | CACUGGCUCCUUUCUGGGUAGA | 10 |
| 11 | hsa-miR-19b-1-5p | AGUUUUGCAGGUUUGCAUCCAGC | 11 |
| 12 | hsa-miR-489 | GUGACAUCACAUAUACGGCAGC | 12 |
| 13 | hsa-miR-192-3p | CUGCCAAUUCCAUAGGUCACAG | 13 |
| 14 | hsa-miR-148b-5p | AAGUUCUGUUAUACACUCAGGC | 14 |
| 15 | hsa-miR-552 | AACAGGUGACUGGUUAGACAA | 15 |
| 16 | hsa-miR-491-3p | CUUAUGCAAGAUUCCCUUCUAC | 16 |
| 17 | hsa-miR-1231 | GUGUCUGGGCGGACAGCUGC | 17 |
| 18 | hsa-miR-517c-3p | AUCGUGCAUCCUUUUAGAGUGU | 18 |
| 19 | hsa-miR-517b-3p; | AUCGUGCAUCCCUUUAGAGUGU | 19 |
| | hsa-miR-517a-3p | | |
| 20 | hsa-miR-1262 | AUGGGUGAAUUUGUAGAAGGAU | 20 |
| 21 | hsa-miR-380-5p | UGGUUGACCAUAGAACAUGCGC | 21 |
| 22 | hsa-miR-1256 | AGGCAUUGACUUCUCACUAGCU | 22 |
| 23 | hsa-miR-1250 | ACGGUGCUGGAUGUGGCCUUU | 23 |
| 24 | hsa-miR-92a-1-5p | AGGUUGGGAUCGGUUGCAAUGCU | 24 |
| 25 | hsa-miR-1253 | AGAGAAGAAGAUCAGCCUGCA | 25 |
| 26 | hsa-miR-326 | CCUCUGGGCCCUUCCUCCAG | 26 |
| 27 | hsa-miR-135b-5p | UAUGGCUUUUCAUUCCUAUGUGA | 27 |
| 28 | hsa-miR-625-5p | AGGGGGAAAGUUCUAUAGUCC | 28 |
| 29 | hsa-miR-363-5p | CGGGUGGAUCACGAUGCAAUUU | 29 |
| 30 | hsa-miR-376a-3p | AUCAUAGAGGAAAAUCCACGU | 30 |
| 31 | hsa-miR-1286 | UGCAGGACCAAGAUGAGCCCU | 31 |
| 32 | hsa-miR-620 | AUGGAGAUAGAUAUAGAAAU | 32 |
| 33 | hsa-miR-506-3p | UAAGGCACCCUUCUGAGUAGA | 33 |
| 34 | hsa-miR-518a-5p; | CUGCAAAGGGAAGCCCUUUC | 34 |
| | hsa-miR-527 | | |
| 35 | hsa-miR-630 | AGUAUUCUGUACCAGGGAAGGU | 35 |
| 36 | hsa-miR-629-3p | GUUCUCCCAACGUAAGCCCAGC | 36 |
| 37 | hsa-miR-532-5p | CAUGCCUUGAGUGUAGGACCGU | 37 |
| 38 | hsa-miR-192-5p | CUGACCUAUGAAUUGACAGCC | 38 |
| 39 | hsa-miR-1275 | GUGGGGGAGAGGCUGUC | 39 |
| 40 | hsa-miR-193b-3p | AACUGGCCCUCAAAGUCCCGCU | 40 |
| 41 | hsa-let-7g-3p | CUGUACAGGCCACUGCCUUGC | 41 |
| 42 | hsa-miR-135a-5p | UAUGGCUUUUUAUUCCUAUGUGA | 42 |
| 43 | hsa-miR-214-5p | UGCCUGUCUACACUUGCUGUGC | 43 |
| 44 | hsa-miR-637 | ACUGGGGGCUUUCGGGCUCUGCGU | 44 |
| 45 | hsa-miR-555 | AGGGUAAGCUGAACCUCUGAU | 45 |
| 46 | hsa-miR-342-5p | AGGGGUGCUAUCUGUGAUUGA | 46 |
| 47 | hsa-miR-362-3p | AACACACCUAUUCAAGGAUUCA | 47 |
| 48 | hsa-miR-671-3p | UCCGGUUCUCAGGGCUCCACC | 48 |
| 49 | hsa-miR-1201 | AGCCUGAUUAAACACAUGCUCUGA | 49 |
| 50 | hsa-miR-15a-3p | CAGGCCAUAUUGUGCUGCCUCA | 50 |
| 51 | hsa-miR-193a-3p | AACUGGCCUACAAAGUCCCAGU | 51 |
| 52 | hsa-miR-604 | AGGCUGCGGAAUUCAGGAC | 52 |
| 53 | hsa-miR-1306-3p | ACGUUGGCUCUGGUGGUG | 53 |
| 54 | hsa-miR-708-5p | AAGGAGCUUACAAUCUAGCUGGG | 54 |
| 55 | hsa-miR-450b-3p | UUGGGAUCAUUUUGCAUCCAUA | 55 |
| 56 | hsa-miR-376a-5p | GUAGAUUCUCCUUCUAUGAGUA | 56 |
| 57 | hsa-miR-185-3p | AGGGGCUGGCUUUCCUCUGGUC | 57 |
| 58 | hsa-miR-541-3p | UGGUGGGCACAGAAUCUGGACU | 58 |
| 59 | hsa-miR-1977 | GAUUAGGGUGCUUAGCUGUUAA | 59 |
| 60 | hsa-miR-593-3p | UGUCUCUGCUGGGGUUUCU | 60 |
| 61 | hsa-miR-24-2-5p | UGCCUACUGAGCUGAAACACAG | 61 |
| 62 | hsa-miR-624-3p | CACAAGGUAUUGGUAUUACCU | 62 |
| 63 | hsa-miR-665 | ACCAGGAGGCUGAGGCCCCU | 63 |
| 64 | hsa-miR-136-3p | CAUCAUCGUCUCAAAUGAGUCU | 64 |
| 65 | hsa-miR-1270 | CUGGAGAUAUGGAAGAGCUGUGU | 65 |
| 66 | hsa-miR-431-5p | UGUCUUGCAGGCCGUCAUGCA | 66 |
| 67 | hsa-miR-452-3p | CUCAUCUGCAAAGAAGUAAGUG | 67 |
| 68 | hsa-miR-202-3p | AGAGGUAUAGGGCAUGGGAA | 68 |
| 69 | hsa-miR-96-5p | UUUGGCACUAGCACAUUUUUGCU | 69 |
| 70 | hsa-miR-185-5p | UGGAGAGAAAGGCAGUUCCUGA | 70 |
| 71 | hsa-miR-744-5p | UGCGGGGCUAGGGCUAACAGCA | 71 |
| 72 | hsa-miR-340-5p | UUAUAAAGCAAUGAGACUGAUU | 72 |
| 73 | hsa-miR-647 | GUGGCUGCACUCACUUCCUUC | 73 |
| 74 | hsa-miR-940 | AAGGCAGGGCCCCCGCUCCCC | 74 |
| 75 | hsa-miR-1295a | UUAGGCCGCAGAUCUGGGUGA | 75 |
| 76 | hsa-miR-1294 | UGUGAGGUUGGCAUUGUUGUCU | 76 |
| 77 | hsa-miR-135b-3p | AUGUAGGGCUAAAAGCCAUGGG | 77 |
| 78 | hsa-miR-608 | AGGGGUGGUGUUGGGACAGCUCCGU | 78 |
| 79 | hsa-miR-2110 | UUGGGGAAACGGCCGCUGAGUG | 79 |
| 80 | hsa-miR-497-5p | CAGCAGCACACUGUGGUUUGU | 80 |
| 81 | hsa-miR-197-3p | UUCACCACCUUCUCCACCCAGC | 81 |
| 82 | hsa-miR-578 | CUUCUUGUGCUCUAGGAUUGU | 82 |
| 83 | hsa-miR-631 | AGACCUGGCCCAGACCUCAGC | 83 |
| 84 | hsa-miR-891b | UGCAACUUACCUGAGUCAUUGA | 84 |
| 85 | hsa-miR-609 | AGGGUGUUUCUCUCAUCUCU | 85 |
| 86 | hsa-miR-516b-5p | AUCUGGAGGUAAGAAGCACUUU | 86 |
| 87 | hsa-miR-222-5p | CUCAGUAGCCAGUGUAGAUCCU | 87 |
| 88 | hsa-miR-1263 | AUGGUACCCUGGCAUACUGAGU | 88 |
| 89 | hsa-miR-141-5p | CAUCUUCCAGUACAGUGUUGGA | 89 |
| 90 | hsa-miR-769-5p | UGAGACCUCUGGGUUCUGAGCU | 90 |
| 91 | hsa-miR-105-5p | UCAAAUGCUCAGACUCCUGUGGU | 91 |
| 92 | hsa-miR-27a-5p | AGGGCUUAGCUGCUUGUGAGCA | 92 |
| 93 | hsa-miR-367-5p | ACUGUUGCUAAUAUGCAACUCU | 93 |
| 94 | hsa-miR-491-5p | AGUGGGGAACCCUUCCAUGAGG | 94 |
| 95 | hsa-miR-220c | ACACAGGGCUGUUGUGAAGACU | 95 |
| 96 | hsa-let-7f-2-3p | CUAUACAGUCUACUGUCUUUCC | 96 |
| 97 | hsa-miR-548k | AAAAGUACUUGCGGAUUUUGCU | 97 |
| 98 | hsa-miR-617 | AGACUUCCCAUUUGAAGGUGGC | 98 |
| 99 | hsa-miR-486-3p | CGGGGCAGCUCAGUACAGGAU | 99 |
| 100 | hsa-miR-765 | UGGAGGAGAAGGAAGGUGAUG | 100 |
| 101 | hsa-miR-182-3p | UGGUUCUAGACUUGCCAACUA | 101 |
| 102 | hsa-miR-1244 | AAGUAGUUGGUUUGUAUGAGAUGGUU | 102 |
| 103 | hsa-miR-215 | AUGACCUAUGAAUUGACAGAC | 103 |
| 104 | hsa-miR-644a | AGUGUGGCUUUCUUAGAGC | 104 |
| 105 | hsa-miR-376b-3p | CGUGGAUAUUCCUUCUAUGUUU | 105 |
| 106 | hsa-miR-1205 | UCUGCAGGGUUUGCUUUGAG | 106 |
| 107 | hsa-miR-330-5p | UCUCUGGGCCUGUGUCUUAGGC | 107 |
| 108 | hsa-miR-432-3p | CUGGAUGGCUCCUCCAUGUCU | 108 |
| 109 | hsa-miR-1271-5p | CUUGGCACCUAGCAAGCACUCA | 109 |
| 110 | hsa-miR-885-3p | AGGCAGCGGGGUGUAGUGGAUA | 110 |
| 111 | hsa-miR-1827 | UGAGGCAGUAGAUUGAAU | 111 |
| 112 | hsa-miR-22-5p | AGUUCUUCAGUGGCAAGCUUUA | 112 |
| 113 | hsa-miR-944 | AAAUUAUUGUACAUCGGAUGAG | 113 |
| 114 | hsa-miR-612 | GCUGGGCAGGGCUUCUGAGCUCCUU | 114 |
| 115 | hsa-miR-103b | UCAUAGCCCUGUACAAUGCUGCU | 115 |
| 116 | hsa-miR-219-1-3p | AGAGUUGAGUCUGGACGUCCCG | 116 |
| 117 | hsa-miR-101-5p | CAGUUAUCACAGUGCUGAUGCU | 117 |
| 118 | hsa-miR-1285-3p | UCUGGGCAACAAAGUGAGACCU | 118 |
| 119 | hsa-miR-1293 | UGGGUGGUCUGGAGAUUUGUGC | 119 |
| 120 | hsa-miR-499a-3p | AACAUCACAGCAAGUCUGUGCU | 120 |
| 121 | hsa-miR-208a | AUAAGACGAGCAAAAAGCUUGU | 121 |
| 122 | hsa-miR-1185-5p | AGAGGAUACCCUUUGUAUGUU | 122 |
| 123 | hsa-miR-1302 | UUGGGACAUACUUAUGCUAAA | 123 |
| 124 | hsa-miR-16-5p | UAGCAGCACGUAAAUAUUGGCG | 124 |
| 125 | hsa-miR-28-5p | AAGGAGCUCACAGUCUAUUGAG | 125 |
| 126 | hsa-miR-378a-5p | CUCCUGACUCCAGGUCCUGUGU | 126 |
| 127 | hsa-miR-183-5p | UAUGGCACUGGUAGAAUUCACU | 127 |
| 128 | hsa-miR-329 | AACACACCUGGUUAACCUCUUU | 128 |
| 129 | hsa-miR-571 | UGAGUUGGCCAUCUGAGUGAG | 129 |
| 130 | hsa-let-7g-5p | UGAGGUAGUAGUUUGUACAGUU | 130 |
| 131 | hsa-miR-764 | GCAGGUGCUCACUUGUCCUCCU | 131 |
| 132 | hsa-miR-628-5p | AUGCUGACAUAUUUACUAGAGG | 132 |
| 133 | hsa-miR-1300 | UUGAGAAGGAGGCUGCUG | 133 |
| 134 | hsa-miR-2115-3p | CAUCAGAAUUCAUGGAGGCUAG | 134 |
| 135 | hsa-miR-510 | UACUCAGGAGAGUGGCAAUCAC | 135 |
| 136 | hsa-miR-1909-3p | CGCAGGGGCCGGGUGCUCACCG | 136 |
| 137 | hsa-miR-1322 | GAUGAUGCUGCUGAUGCUG | 137 |
| 138 | hsa-miR-511 | GUGUCUUUUGCUCUGCAGUCA | 138 |
| 139 | hsa-miR-3115 | AUAUGGGUUUACUAGUUGGU | 139 |
| 140 | hsa-miR-3116 | UGCCUGGAACAUAGUAGGGACU | 140 |
| 141 | hsa-miR-3119 | UGGCUUUUAACUUUGAUGGC | 141 |
| 142 | hsa-miR-93-3p | ACUGCUGAGCUAGCACUUCCCG | 142 |
| 143 | hsa-miR-3125 | UAGAGGAAGCUGUGGAGAGA | 143 |
| 144 | hsa-miR-3127-5p | AUCAGGGCUUGUGGAAUGGGAAG | 144 |
| 145 | hsa-miR-323a-5p | AGGUGGUCCGUGGCGCGUUCGC | 145 |
| 146 | hsa-miR-646 | AAGCAGCUGCCUCUGAGGC | 146 |
| 147 | hsa-miR-449b-5p | AGGCAGUGUAUUGUUAGCUGGC | 147 |
| 148 | hsa-miR-181c-3p | AACCAUCGACCGUUGAGUGGAC | 148 |
| 149 | hsa-miR-16-1-3p | CCAGUAUUAACUGUGCUGCUGA | 149 |
| 150 | hsa-miR-744-3p | CUGUUGCCACUAACCUCAACCU | 150 |
| 151 | hsa-miR-585 | UGGGCGUAUCUGUAUGCUA | 151 |
| 152 | hsa-miR-34b-5p | UAGGCAGUGUCAUUAGCUGAUUG | 152 |
| 153 | hsa-miR-766-3p | ACUCCAGCCCCACAGCCUCAGC | 153 |
| 154 | hsa-miR-147a | GUGUGUGGAAAUGCUUCUGC | 154 |
| 155 | hsa-miR-3139 | UAGGAGCUCAACAGAUGCCUGUU | 155 |
| 156 | hsa-miR-3140-3p | AGCUUUUGGGAAUUCAGGUAGU | 156 |
| 157 | hsa-miR-126-3p | UCGUACCGUGAGUAAUAAUGCG | 157 |
| 158 | hsa-miR-221-5p | ACCUGGCAUACAAUGUAGAUUU | 158 |
| 159 | hsa-miR-1261 | AUGGAUAAGGCUUUGGCUU | 159 |
| 160 | hsa-miR-421 | AUCAACAGACAUUAAUUGGGCGC | 160 |
| 161 | hsa-miR-3144-3p | AUAUACCUGUUCGGUCUCUUUA | 161 |
| 162 | hsa-miR-888-3p | GACUGACACCUCUUUGGGUGAA | 162 |
| 163 | hsa-miR-629-5p | UGGGUUUACGUUGGGAGAACU | 163 |
| 164 | hsa-miR-576-3p | AAGAUGUGGAAAAAUUGGAAUC | 164 |
| 165 | hsa-miR-25-5p | AGGCGGAGACUUGGGCAAUUG | 165 |
| 166 | hsa-miR-3150a-3p | CUGGGGAGAUCCUCGAGGUUGG | 166 |
| 167 | hsa-miR-129-5p | CUUUUUGCGGUCUGGGCUUGC | 167 |
| 168 | hsa-miR-3154 | CAGAAGGGGAGUUGGGAGCAGA | 168 |
| 169 | hsa-miR-4312 | GGCCUUGUUCCUGUCCCCA | 169 |
| 170 | hsa-miR-3157-5p | UUCAGCCAGGCUAGUGCAGUCU | 170 |
| 171 | hsa-miR-3158-3p | AAGGGCUUCCUCUCUGCAGGAC | 171 |
| 172 | hsa-miR-4315 | CCGCUUUCUGAGCUGGAC | 172 |
| 173 | hsa-miR-3160-3p | AGAGCUGAGACUAGAAAGCCCA | 173 |
| 174 | hsa-miR-4314 | CUCUGGGAAAUGGGACAG | 174 |
| 175 | hsa-miR-3911 | UGUGUGGAUCCUGGAGGAGGCA | 175 |
| 176 | hsa-miR-3616-3p | CGAGGGCAUUUCAUGAUGCAGGC | 176 |
| 177 | hsa-miR-3618 | UGUCUACAUUAAUGAAAAGAGC | 177 |
| 178 | hsa-miR-3619-5p | UCAGCAGGCAGGCUGGUGCAGC | 178 |
| 179 | hsa-miR-3916 | AAGAGGAAGAAAUGGCUGGUUCUCAG | 179 |
| 180 | hsa-miR-3918 | ACAGGGCCGCAGAUGGAGACU | 180 |
| 181 | hsa-miR-3150b-3p | UGAGGAGAUCGUCGAGGUUGG | 181 |
| 182 | hsa-miR-3165 | AGGUGGAUGCAAUGUGACCUCA | 182 |
| 183 | hsa-miR-3166 | CGCAGACAAUGCCUACUGGCCUA | 183 |
| 184 | hsa-miR-4256 | AUCUGACCUGAUGAAGGU | 184 |
| 185 | hsa-miR-3170 | CUGGGGUUCUGAGACAGACAGU | 185 |
| 186 | hsa-miR-4259 | CAGUUGGGUCUAGGGGUCAGGA | 186 |
| 187 | hsa-miR-3173-3p | AAAGGAGGAAAUAGGCAGGCCA | 187 |
| 188 | hsa-miR-1193 | GGGAUGGUAGACCGGUGACGUGC | 188 |
| 189 | hsa-miR-3622b-5p | AGGCAUGGGAGGUCAGGUGA | 189 |
| 190 | hsa-miR-3923 | AACUAGUAAUGUUGGAUUAGGG | 190 |
| 191 | hsa-miR-3927-3p | CAGGUAGAUAUUUGAUAGGCAU | 191 |
| 192 | hsa-miR-3174 | UAGUGAGUUAGAGAUGCAGAGCC | 192 |
| 193 | hsa-miR-4325 | UUGCACUUGUCUCAGUGA | 193 |
| 194 | hsa-miR-3179 | AGAAGGGGUGAAAUUUAAACGU | 194 |
| 195 | hsa-miR-3180-3p | UGGGGCGGAGCUUCCGGAGGCC | 195 |
| 196 | hsa-miR-4265 | CUGUGGGCUCAGCUCUGGG | 196 |
| 197 | hsa-miR-3653 | CUAAGAAGUUGACUGAAG | 197 |
| 198 | hsa-miR-3936 | UAAGGGGUGUAUGGCAGAUGCA | 198 |
| 199 | hsa-miR-4268 | GGCUCCUCCUCUCAGGAUGUG | 199 |
| 200 | hsa-miR-3186-3p | UCACGCGGAGAGAUGGCUUUG | 200 |
| 201 | hsa-miR-4270 | UCAGGGAGUCAGGGGAGGGC | 201 |
| 202 | hsa-miR-3661 | UGACCUGGGACUCGGACAGCUG | 202 |
| 203 | hsa-miR-3665 | AGCAGGUGCGGGGCGGCG | 203 |
| 204 | hsa-miR-320e | AAAGCUGGGUUGAGAAGG | 204 |
| 205 | hsa-miR-4276 | CUCAGUGACUCAUGUGC | 205 |
| 206 | hsa-miR-3190-3p | UGUGGAAGGUAGACGGCCAGAGA | 206 |
| 207 | hsa-miR-3192 | UCUGGGAGGUUGUAGCAGUGGAA | 207 |
| 208 | hsa-miR-3672 | AUGAGACUCAUGUAAAACAUCUU | 208 |
| 209 | hsa-miR-3675-5p | UAUGGGGCUUCUGUAGAGAUUUC | 209 |
| 210 | hsa-miR-4283 | UGGGGCUCAGCGAGUUU | 210 |
| 211 | hsa-miR-3200-3p | CACCUUGCGCUACUCAGGUCUG | 211 |
| 212 | hsa-miR-4290 | UGCCCUCCUUUCUUCCCUC | 212 |
| 213 | hsa-miR-3126-3p | CAUCUGGCAUCCGUCACACAGA | 213 |
| 214 | hsa-miR-3682-3p | UGAUGAUACAGGUGGAGGUAG | 214 |
| 215 | hsa-miR-4297 | UGCCUUCCUGUCUGUG | 215 |
| 216 | hsa-miR-3605-5p | UGAGGAUGGAUAGCAAGGAAGCC | 216 |
| 217 | hsa-miR-4306 | UGGAGAGAAAGGCAGUA | 217 |
| 218 | hsa-miR-3180 | UGGGGCGGAGCUUCCGGAG | 218 |
| 219 | hsa-miR-3907 | AGGUGCUCCAGGCUGGCUCACA | 219 |

TABLE 2
Synthetic lethal miRNAs in neuroblastoma cells

| | | | SEQ ID |
|---|---|---|---|
| Row | mature miRNA | mature miRNA sequence | NO |
| 1 | hsa-miR-1321 | CAGGGAGGUGAAUGUGAU | 220 |
| 2 | hsa-miR-99b-5p | CACCCGUAGAACCGACCUUGCG | 221 |
| 3 | hsa-miR-100-5p | AACCCGUAGAUCCGAACUUGUG | 222 |
| 4 | hsa-miR-3151 | GGUGGGGCAAUGGGAUCAGGU | 223 |
| 5 | hsa-miR-3181 | AUCGGGCCCUCGGCGCCGG | 224 |
| 6 | hsa-miR-34a-5p | UGGCAGUGUCUUAGCUGGUUGU | 225 |
| 7 | hsa-miR-34c-5p | AGGCAGUGUAGUUAGCUGAUUGC | 226 |
| 8 | hsa-miR-3646 | AAAAUGAAAUGAGCCCAGCCCA | 227 |
| 9 | hsa-miR-380-3p | UAUGUAAUAUGGUCCACAUCUU | 228 |
| 10 | hsa-miR-3924 | AUAUGUAUAUGUGACUGCUACU | 229 |
| 11 | hsa-miR-449a | UGGCAGUGUAUUGUUAGCUGGU | 230 |
| 12 | hsa-miR-485-3p | GUCAUACACGGCUCUCCUCUCU | 231 |
| 13 | hsa-miR-515-3p | GAGUGCCUUCUUUUGGAGCGUU | 232 |
| 14 | hsa-miR-299-3p | UAUGUGGGAUGGUAAACCGCUU | 233 |
| 15 | hsa-let-7b-3p | CUAUACAACCUACUGCCUUCCC | 234 |
| 16 | hsa-miR-125a-3p | ACAGGUGAGGUUCUUGGGAGCC | 235 |
| 17 | hsa-miR-150-5p | UCUCCCAACCCUUGUACCAGUG | 236 |
| 18 | hsa-miR-1909-5p | UGAGUGCCGGUGCCUGCCCUG | 237 |
| 19 | hsa-miR-190b | UGAUAUGUUUGAUAUUGGGUU | 238 |
| 20 | hsa-miR-1911-5p | UGAGUACCGCCAUGUCUGUUGGG | 239 |
| 21 | hsa-miR-1978 | GGUUUGGUCCUAGCCUUUCUA | 240 |
| 22 | hsa-miR-199b-5p | CCCAGUGUUUAGACUAUCUGUUC | 241 |
| 23 | hsa-miR-2116-3p | CCUCCCAUGCCAAGAACUCCC | 242 |
| 24 | hsa-miR-216b | AAAUCUCUGCAGGCAAAUGUGA | 243 |
| 25 | hsa-miR-29b-2-5p | CUGGUUUCACAUGGUGGCUUAG | 244 |
| 26 | hsa-miR-302b-3p | UAAGUGCUUCCAUGUUUUAGUAG | 245 |
| 27 | hsa-miR-3137 | UCUGUAGCCUGGGAGCAAUGGGGU | 246 |
| 28 | hsa-miR-3147 | GGUUGGGCAGUGAGGAGGGUGUGA | 247 |
| 29 | hsa-miR-3161 | CUGAUAAGAACAGAGGCCCAGAU | 248 |
| 30 | hsa-miR-3162-5p | UUAGGGAGUAGAAGGGUGGGGAG | 249 |
| 31 | hsa-miR-3164 | UGUGACUUUAAGGGAAAUGGCG | 250 |
| 32 | hsa-miR-3175 | CGGGGAGAGAACGCAGUGACGU | 251 |
| 33 | hsa-miR-3199 | AGGGACUGCCUUAGGAGAAAGUU | 252 |
| 34 | hsa-miR-372 | AAAGUGCUGCGACAUUUGAGCGU | 253 |
| 35 | hsa-miR-379-5p | UGGUAGACUAUGGAACGUAGG | 254 |
| 36 | hsa-miR-3934-5p | UCAGGUGUGGAAACUGAGGCAG | 255 |
| 37 | hsa-miR-410 | AAUAUAACACAGAUGGCCUGU | 256 |
| 38 | hsa-miR-4264 | ACUCAGUCAUGGUCAUU | 257 |
| 39 | hsa-miR-4274 | CAGCAGUCCCUCCCCCUG | 258 |
| 40 | hsa-miR-4296 | AUGUGGGCUCAGGCUCA | 259 |
| 41 | hsa-miR-4302 | CCAGUGUGGCUCAGCGAG | 260 |
| 42 | hsa-miR-513c-5p | UUCUCAAGGAGGUGUCGUUUAU | 261 |
| 43 | hsa-miR-516a-3p; | UGCUUCCUUUCAGAGGGU | 262 |
| | hsa-miR-516b-3p | | |
| 44 | hsa-miR-518c-5p | UCUCUGGAGGGAAGCACUUUCUG | 263 |
| 45 | hsa-miR-520a-3p | AAAGUGCUUCCCUUUGGACUGU | 264 |
| 46 | hsa-miR-520b | AAAGUGCUUCCUUUUAGAGGG | 265 |
| 47 | hsa-miR-520d-3p | AAAGUGCUUCUCUUUGGUGGGU | 266 |
| 48 | hsa-miR-520e | AAAGUGCUUCCUUUUUGAGGG | 267 |
| 49 | hsa-miR-625-3p | GACUAUAGAACUUUCCCCCUCA | 268 |
| 50 | hsa-miR-648 | AAGUGUGCAGGGCACUGGU | 269 |
| 51 | hsa-miR-652-3p | AAUGGCGCCACUAGGGUUGUG | 270 |
| 52 | hsa-miR-662 | UCCCACGUUGUGGCCCAGCAG | 271 |

In one example, the RNAi molecule is a lethal RNAi molecule. For example, the RNAi molecule is a lethal RNAi molecule selected from the group consisting of:
(i) a miRNA selected from the group of miRNAs set forth in Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the RNAi molecule may be a lethal RNAi molecule selected from the group consisting of:
(i) a miRNA selected from the group of miRNAs set forth in rows 1-20 of Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

An exemplary lethal RNAi molecule contemplated for use in the treatment of neuroblastoma is a miRNA selected from the group of miRNAs set forth in rows 1-20 of Table 1.

An exemplary lethal RNAi molecule contemplated for use in the treatment of neuroblastoma is a miRNA of the miR-515 family which is selected from the group of miRNAs set forth in Table 1.

Other exemplary lethal RNAi molecules contemplated for use in the treatment of neuroblastoma may be selected from the group consisting of:
(i) a miRNA selected from the group consisting of miR-516b-5p, miR-517a-3p, miR-517c-3p, miR-518b, miR-519e-5p, and miR-526b-5p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

In one example, the lethal RNAi molecule is a miRNA selected from the group consisting of miR-516b-5p, miR-517a-3p, miR-517c-3p, miR-518b, miR-519e-5p and miR-526b-5p.

In one example, the lethal RNAi molecule is a miRNA comprising an effector sequence which targets the same mRNA transcript sequence as an effector sequence of a miRNA selected from the group consisting of miR-516b-5p, miR-517a-3p, miR-517c-3p, miR-518b, miR-519e-5p and miR-526b-5p.

In one example, the lethal RNAi molecule is a pri-miRNA corresponding to a miRNA selected from the group consisting of miR-516b-5p, miR-517a-3p, miR-517c-3p, miR-518b, miR-519e-5p and miR-526b-5p.

In one example, the lethal RNAi molecule is a pre-miRNA corresponding to a miRNA selected from the group consisting of miR-516b-5p, miR-517a-3p, miR-517c-3p, miR-518b, miR-519e-5p and miR-526b-5p.

In one example, the lethal RNAi molecule is a miRNA mimic corresponding to a miRNA selected from the group consisting of miR-516b-5p, miR-517a-3p, miR-517c-3p, miR-518b, miR-519e-5p and miR-526b-5p.

In one example, the lethal RNAi molecule is a miRNA selected from the group consisting of miR-516b-5p, miR-517a-3p, miR-517c-3p, miR-518b, miR-519e-5p and miR-526b-5p, which has a chemical modification or a modified nucleobase.

In one example, the lethal RNAi molecule is a nucleic acid encoding a miRNA selected from miR-516b-5p, miR-517a-3p, miR-517c-3p, miR-518b, miR-519e-5p, and miR-526b-5p or a corresponding pri-miRNA or pre-miRNA thereof.

In one example, the lethal RNAi molecule for use in accordance with the present disclosure is an RNAi molecule corresponding to miR-516b-5p as described in any example hereof.

In one example, the lethal RNAi molecule for use in accordance with the present disclosure is an RNAi molecule corresponding to miR-517a-3p as described in any example hereof.

In one example, the lethal RNAi molecule for use in accordance with the present disclosure is an RNAi molecule corresponding to miR-517c-3p as described in any example hereof.

In one example, the lethal RNAi molecule for use in accordance with the present disclosure is an RNAi molecule corresponding to miR-518b as described in any example hereof.

In one example, the lethal RNAi molecule for use in accordance with the present disclosure is an RNAi molecule corresponding to miR-519e-5p as described in any example hereof.

In one example, the lethal RNAi molecule for use in accordance with the present disclosure is an RNAi molecule corresponding to miR-526b-5p as described in any example hereof.

In another example, the RNAi molecule contemplated for use in the treatment of neuroblastoma is a synthetic lethal RNAi molecule. Synthetic lethal RNAi molecules of the disclosure are contemplated for use in the treatment of neuroblastoma in combination with a chemotherapeutic agent. For example, the RNAi molecule may be a synthetic lethal RNAi molecule selected from the group consisting of:
(i) a miRNA selected from the group of miRNAs set forth in Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

For example, the RNAi molecule may be a synthetic lethal RNAi molecule selected from the group consisting of:
(i) a miRNA selected from the group of miRNAs set forth in rows 1-13 of Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

An exemplary synthetic lethal RNAi molecule contemplated for use in the treatment of neuroblastoma in combination with a chemotherapeutic agent is a miRNA selected from the group of miRNAs set forth in rows 1-13 of Table 2.

An exemplary synthetic lethal RNAi molecule contemplated for use in the treatment of neuroblastoma in combination with a chemotherapeutic agent is a miRNA of the miR-515 family which is selected from the group of miRNAs set forth in Table 2.

Other exemplary synthetic lethal RNAi molecules contemplated for use in the treatment of neuroblastoma in combination with a chemotherapeutic agent may be selected from the group consisting of:
(i) a miRNA selected from the group consisting of miR-380-3p, miR-99b-5p and miR-485-3p;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

In one example, the synthetic lethal RNAi molecule is a miRNA selected from the group consisting of miR-380-3p, miR-99b-5p and miR-485-3p.

In one example, the synthetic lethal RNAi molecule is a miRNA comprising an effector sequence which targets the same mRNA transcript sequence as an effector sequence of a miRNA selected from the group consisting of miR-380-3p, miR-99b-5p and miR-485-3p.

In one example, the synthetic lethal RNAi molecule is a pri-miRNA corresponding to a miRNA selected from the group consisting of miR-380-3p, miR-99b-5p and miR-485-3p.

In one example, the synthetic lethal RNAi molecule is a pre-miRNA corresponding to a miRNA selected from the group consisting of miR-380-3p, miR-99b-5p and miR-485-3p.

In one example, the synthetic lethal RNAi molecule is a miRNA mimic corresponding to a miRNA selected from the group consisting of miR-380-3p, miR-99b-5p and miR-485-3p.

In one example, the synthetic lethal RNAi molecule is a miRNA selected from the group consisting of miR-380-3p, miR-99b-5p and miR-485-3p, which has a chemical modification or a modified nucleobase.

In one example, the synthetic lethal RNAi molecule is a nucleic acid encoding a miRNA selected from miR-380-3p, miR-99b-5p and miR-485-3p or a corresponding pri-miRNA or pre-miRNA thereof.

In one example, the synthetic lethal RNAi molecule for use in accordance with the present disclosure is an RNAi molecule corresponding to miR-380-3p as described in any example hereof.

In one example, the synthetic lethal RNAi molecule for use in accordance with the present disclosure is an RNAi molecule corresponding to miR-99b-5p as described in any example hereof.

In one example, the synthetic lethal RNAi molecule for use in accordance with the present disclosure is an RNAi molecule corresponding to miR-485-3p as described in any example hereof.

In one example, the RNAi molecule for use in the method of the disclosure is a miRNA selected from the group of miRNAs set forth in Table 1 or Table 2, or a corresponding pri-miRNA, pre-miRNA or miRNA mimic thereof.

In one example, the RNAi molecule is a lethal RNAi moleculeselected from the group of lethal miRNAs set forth in Table 1 or the corresponding pri-miRNA, pre-miRNA or miRNA mimic thereof. For example, the RNAi molecule may be selected from the group of lethal miRNAs set forth in rows 1-20 of Table 1 or the corresponding pri-miRNA, pre-miRNA or miRNA mimic thereof. For example, the RNAi molecule may be from the miR-515 family and selected from the group of lethal miRNAs set forth in Table 1 or the corresponding pri-miRNA, pre-miRNA or miRNA mimic thereof. In one example, the RNAi molecule may be selected from the group of lethal RNAi molecules consisting of miR-516b-5p, miR-517a-3p, miR-517c-3p, miR-518b, miR-519e-5p and miR-526b-5p or the corresponding pri-miRNA, pre-miRNA or miRNA mimic thereof. In one example, the lethal RNAi molecule is miR-516b-5p or the corresponding pri-miRNA, pre-miRNA or miRNA mimic thereof. In one example, the lethal RNAi molecule is miR-517a-3p or the corresponding pri-miRNA, pre-miRNA or miRNA mimic thereof. In one example, the lethal RNAi molecule is miR-517c-3p or the corresponding pri-miRNA, pre-miRNA or miRNA mimic thereof. In one example, the lethal RNAi molecule is miR-518b or the corresponding pri-miRNA, pre-miRNA or miRNA mimic thereof. In one example, the lethal RNAi molecule is miR-519e-5p or the corresponding pri-miRNA, pre-miRNA or miRNA mimic thereof. In one example, the lethal RNAi molecule is miR-526b-5p or the corresponding pri-miRNA, pre-miRNA or miRNA mimic thereof.

In one example, the RNAi molecule is a synthetic lethal RNAi molecule selected from the group of synthetic lethal miRNAs set forth in Table 2 or the corresponding pri-miRNA, pre-miRNA or miRNA mimic thereof. For example, the RNAi molecule may be selected from the group of synthetic lethal miRNAs set forth in rows 1-13 of Table 2 or the corresponding pri-miRNA, pre-miRNA or miRNA mimic thereof. For example, the RNAi molecule may be from the miR-515 family and selected from the group of synthetic lethal miRNAs set forth in Table 2 or the corresponding pri-miRNA, pre-miRNA or miRNA mimic thereof. In one example, the RNAi molecule may be a synthetic lethal RNAi molecule selected from the group consisting of miR-380-3p, miR-99b-5p and miR-485-3p or the corresponding pri-miRNA, pre-miRNA or miRNA mimic thereof. In one example, the synthetic lethal RNAi molecule is miR-380-3p or the corresponding pri-miRNA, pre-miRNA or miRNA mimic thereof. In one example, the synthetic lethal RNAi molecule is miR-99b-5p or the corresponding pri-miRNA, pre-miRNA or miRNA mimic thereof. In one example, the synthetic lethal RNAi molecule is miR-485-3p or the corresponding pri-miRNA, pre-miRNA or miRNA mimic thereof.

In one example, the RNAi molecule for use in the method of the disclosure is a miRNA, or corresponding pri-miRNA, pre-miRNA or miRNA mimic, comprising an effector sequence of a miRNA set forth in Table 1 or Table 2. For example, the miRNA, pri-miRNA, pre-miRNA or miRNA mimic may comprise an effector sequence of a lethal miRNAs set forth in Table 1 or is the corresponding pri-miRNA or pri-miRNA thereof. For example, the miRNA, pri-miRNA, pre-miRNA or miRNA mimic may comprise an effector sequence of a lethal miRNAs set forth in rows 1-20 of Table 1. For example, the miRNA, pri-miRNA, pre-miRNA or miRNA mimic may comprise an effector sequence of miR-515 family member which is a lethal miRNA set forth in Table 1. For example, the miRNA, pri-miRNA, pre-miRNA or miRNA mimic may comprise an effector sequence of a lethal miRNA selected from the group of lethal RNAi molecules consisting of miR-516b-5p, miR-517a-3p, miR-517c-3p, miR-518b, miR-519e-5p and miR-526b-5p. For example, the miRNA, pri-miRNA, pre-miRNA or miRNA mimic may comprise an effector sequence of a synthetic lethal miRNAs set forth in Table 2. For example, the miRNA, pri-miRNA, pre-miRNA or miRNA mimic may comprise an effector sequence of a synthetic lethal miRNA set forth in rows 1-13 of Table 2. For example, the miRNA, pri-miRNA, pre-miRNA or miRNA mimic may comprise an effector sequence of miR-515 family member which is a synthetic lethal miRNA set forth in Table 2. For example, the miRNA, pri-miRNA, pre-miRNA or miRNA mimic may comprise an effector sequence of a synthetic lethal miRNA selected from the group of synthetic lethal RNAi molecules consisting of miR-380-3p, miR-99b-5p and miR-485-3p.

In one example, the RNAi molecule for use in the method of the disclosure is a miRNA, or corresponding pri-miRNA, pre-miRNA or miRNA mimic, comprising an effector sequence which targets the same mRNA transcript sequence as an effector sequence of a miRNA set forth in Table 1 or Table 2 or a corresponding pri-miRNA or pri-miRNA thereof. In this regard, the RNAi molecule may be a miRNA from the same miRNA family as a miRNA set forth in Table 1 or Table 2. As used herein, the term miRNA family” refers to a group a miRNA species that share identity across at least 6 consecutive nucleotides, also referred to as the “seed sequence”, as described in Brennecke et al., (2005) PLoS Biol 3(3):pe85. As used herein, the “seed sequence” denotes nucleotides at positions 1-6, 1-7, 2-7 or 2-8 of the effector sequence of a miRNA sequence. This is typically located at the 5′ end of the miRNA effector sequence. Accordingly, a miRNA, or corresponding pri-miRNA, pre-miRNA or miRNA mimic, comprising an effector sequence which targets the same mRNA transcript sequence as an effector sequence of a miRNA set forth in Table 1 or Table 2, will comprise a seed sequence of a miRNA set forth in Table 1 or Table 2.

In one example, the miRNA, pri-miRNA, pre-miRNA or miRNA mimic may comprise an effector sequence which targets the same mRNA transcript sequence as an effector sequence of a lethal miRNA set forth in Table 1. For example, the miRNA, pri-miRNA, pre-miRNA or miRNA mimic may comprise an effector sequence which targets the same mRNA transcript sequence as an effector sequence of a lethal miRNA set forth in rows 1-20 of Table 1. For example, the miRNA, pri-miRNA, pre-miRNA or miRNA mimic may comprise an effector sequence which targets the same mRNA transcript sequence as an effector sequence of a miR-515 family member which is a lethal miRNA set forth in Table 1. For example, the miRNA, pri-miRNA, pre-miRNA or miRNA mimic may comprise an effector sequence which targets the same mRNA transcript sequence as an effector sequence of a lethal miRNA selected from the group of consisting of miR-516b-5p, miR-517a-3p, miR-517c-3p, miR-518b, miR-519e-5p and miR-526b-5p. In one example, the miRNA, pri-miRNA, pre-miRNA or miRNA mimic comprises an effector sequence which targets the same mRNA transcript sequence as the effector sequence of miR-516b-5p. In one example, the miRNA, pri-miRNA, pre-miRNA or miRNA mimic comprises an effector sequence which targets the same mRNA transcript sequence as the effector sequence of miR-517a-3p. In one example, the miRNA, pri-miRNA, pre-miRNA or miRNA mimic comprises an effector sequence which targets the same mRNA transcript sequence as the effector sequence of miR-517c-3p. In one example, the miRNA, pri-miRNA, pre-miRNA or miRNA mimic comprises an effector sequence which targets the same mRNA transcript sequence as the effector sequence of miR-518b. In one example, the miRNA, pri-miRNA, pre-miRNA or miRNA mimic comprises an effector sequence which targets the same mRNA transcript sequence as the effector sequence of miR-519e-5p. In one example, the miRNA, pri-miRNA, pre-miRNA or miRNA mimic comprises an effector sequence which targets the same mRNA transcript sequence as the effector sequence of miR-526b-5p.

In another example, the miRNA, pri-miRNA, pre-miRNA or miRNA mimic may comprise an effector sequence which targets the same mRNA transcript sequence as an effector sequence of a synthetic lethal miRNA set forth in Table 2. For example, the miRNA, pri-miRNA, pre-miRNA or miRNA mimic may comprise an effector sequence which targets the same mRNA transcript sequence as an effector sequence of a synthetic lethal miRNA set forth in rows 1-13 of Table 2. For example, the miRNA, pri-miRNA, pre-miRNA or miRNA mimic may comprise an effector sequence which targets the same mRNA transcript sequence as an effector sequence of a miR-515 family member which is a synthetic lethal miRNA set forth in Table 2. For example, the miRNA, pri-miRNA, pre-miRNA or miRNA mimic may comprise an effector sequence which targets the same mRNA transcript sequence as an effector sequence of a synthetic lethal miRNA selected from the group consisting of miR-380-3p, miR-99b-5p and miR-485-3p. In one example, the miRNA, pri-miRNA, pre-miRNA or miRNA mimic comprises an effector sequence which targets the same mRNA transcript sequence as the effector sequence of miR-380-3p. In one example, the miRNA, pri-miRNA, pre-miRNA or miRNA mimic comprises an effector sequence which targets the same mRNA transcript sequence as the effector sequence of miR-99b-5p. In one example, the miRNA, pri-miRNA, pre-miRNA or miRNA mimic comprises an effector sequence which targets the same mRNA transcript sequence as the effector sequence of miR-485-3p.

RNAi molecules contemplated for use in the method of the disclosure may be either synthetic or expressed by a suitable vector comprising a DNA template for transcription of the RNAi molecule(s).

Synthetic RNAi molecules e.g., miRNAs or precursors thereof and miRNA mimics, may be manufactured by methods known in the art such as by typical oligonucleotide synthesis, and often will incorporate chemical modifications to increase half-life and/or efficacy of the, and/or to allow for a more robust delivery formulation. Many modifications of oligonucleotides are known in the art.

In one example, substantially all of the nucleotides of the RNAi molecule of the disclosure are modified. In other examples, all of the nucleotides of the RNAi molecule of the disclosure are modified. RNAi molecules contemplated for use in the method of the disclosure in which “substantially all of the nucleotides are modified” are largely but not wholly modified and can include not more than 5, 4, 3, 2, or 1 unmodified nucleotides.

In other examples, modifications may be made to the sequence of an RNAi molecule of the disclosure e.g., a miRNA or corresponding pri-miRNA or pre-miRNA, without disrupting miRNA activity. In this way, the RNAi molecule may vary from a corresponding naturally-occurring miRNA sequence, but retain one or more functional characteristics of that miRNA (e.g., enhancement of cancer cell susceptibility to chemotherapeutic agents, inhibition of cancer cell proliferation, induction of cancer cell apoptosis, specific miRNA target inhibition). Such an RNAi molecule may be referred to as a “functional variant” of a naturally-occurring miRNA or corresponding precursor molecule. In some examples, a functional variant retains all of the functional characteristics of the corresponding naturally-occurring miRNA. In certain examples, an RNAi molecule which is a naturally-occurring miRNA has a nucleobase sequence that is a least about 60%, 65%, 70%, 75%, 80%, 85%, 90%, 91%, 92%, 93%, 94%, 95%, 96%, 97%, 98% or 99% identical to the miRNA or corresponding precursor thereof over a region of about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the functional variant hybridizes to the complement of the miRNA or precursor thereof under stringent hybridization conditions. Accordingly, in certain examples, the nucleobase sequence of an RNAi molecule which is a functional variant may be capable of hybridizing to one or more target sequences of the miRNA.

In one example, the or each RNAi molecule contemplated for use in the method of the disclosure comprises one or more overhang regions and/or capping groups at the 3′-end, 5′-end, or both ends of one or both strands of the duplex (if provided as a duplex). The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one example, the nucleotides in the overhang region of the or each RNA each independently are a modified or unmodified nucleotide including, but no limited to 2′-sugar modified, such as, 2-F, 2′-O-methyl, thymidine (T), deoxy-thymine (dT), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, dTdT can be an overhang sequence for either end on either strand. The overhang can form a mismatch with the target mRNA or it can be complementary to the gene sequences being targeted or can be another sequence.

The 5′- or 3′-overhangs at the strand comprising the effector sequence or the strand comprising the effector complement sequence or both strands of the or each RNAi molecule can be phosphorylated. In some examples, the overhang region(s) contains two nucleotides having a phosphorothioate between the two nucleotides, where the two nucleotides can be the same or different.

In one example, the RNAi molecule(s) of the disclosure contain(s) only a single overhang, which can strengthen the interference activity of the RNAi molecule, without affecting its overall stability. For example, the single-stranded overhang is be located at the 3′-terminal end of the effector sequence or, alternatively, at the 3′-terminal end of the effector complement sequence. In one example, the RNAi molecule(s) of the disclosure also comprise(s) a blunt end, located at the 5′-end of the effector complement sequence (or the 3′-end of the effector sequence) or vice versa.

Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. Specific examples of RNAi molecules useful in the method of the disclosure include, but are not limited to RNAi molecules containing modified backbones or no natural internucleoside linkages. RNAi molecules having modified backbones include, among others, those that do not have a phosphorus atom in the backbone. For the purposes of this specification, and as sometimes referenced in the art, modified RNAi molecules that do not have a phosphorus atom in their internucleoside backbone can also be considered to be oligonucleosides. In some example, a modified RNAi molecule will have a phosphorus atom in its internucleoside backbone. Representative U.S. patents that teach the preparation of phosphorus-containing linkages include, but are not limited to, U.S. Pat. Nos. 7,015,315; 7,041,816; 7,273,933; 7,321,029; and U.S. Pat. RE39464.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach exemplary forms of these oligonucleosides include, but are not limited to, U.S. Pat. Nos. 5,663,312; 5,633,360; 5,677,437; and 5,677,439.

In one example, the RNAi molecule of the disclosure comprises only unmodified or natural bases, e.g., a described below.

Modified RNAi molecules of the disclosure can also contain one or more substituted sugar moieties. For example, the RNAi molecules described herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)ₙOCH₃, O(CH₁)ₙNH₂, O(CH₂)ₙCH₃, O(CH₂)ₙONH₂, and O(CH₂)ₙON[(CH₂)ₙCH₃)]₂, where n and m are from 1 to about 10.

In another example, the RNAi molecules useful in the method of the disclosure can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as deoxy-thymine (dT), 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine.

The RNAi molecules described herein can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447). Thus, it may be advantageous for the RNAi molecules of the disclosure e.g., miRNAs and miRNA mimics, to include one or more LNAs to improve serum stability and/or reduce off target effects when treating a subject for neuroblastoma.

Potentially stabilizing modifications to the ends of RNAi molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

In another example, the exemplary RNAi molecule of the disclosure may be provided as a hairpin structure e.g., a pri-miRNA or a pre-miRNA, comprising a stem loop sequence positioned between cognate effector and effector complement sequences such that the RNAi molecule forms a single contiguous sequence. A stem loop sequence should be of sufficient length to permit the effector sequence and the effector complement sequence to anneal to one another. Suitable stem loop sequences may be selected from those known in the art.

In one example, the RNAi molecules used in the method of the disclosure are chemically synthesized. Oligonucleotides (e.g., certain modified oligonucleotides or portions of oligonucleotides lacking ribonucleotides) are synthesized using protocols known in the art, for example as described in Caruthers et al., 1992, Methods in Enzymology 211, 3-19, WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. The synthesis of oligonucleotides makes use of common nucleic acid protecting and coupling groups, such as dimethoxytrityl at the 5′-end, and phosphoramidites at the 3′-end.

RNA without modifications are synthesized using procedures as described in Usman et al., 1987, J. Am. Chem. Soc., 109, 7845; Scaringe et al., 1990, Nucleic Acids Res., 18, 5433. These syntheses makes use of common nucleic acid protecting and coupling groups, such as dimethoxytrityl at the 5′-end, and phosphoramidites at the 3′-end that can be used for certain RNAi molecules of the disclosure.

In certain examples, the RNAi molecules used in the method of the disclosure are synthesized, deprotected, and analyzed according to methods described in U.S. Pat. Nos. 6,995,259, 6,686,463, 6,673,918, 6,649,751, and/or 6,989,442.

In an alternative example, the RNAi molecules used in the method of the disclosure are synthesized as discrete components and joined together post-synthetically, for example, by ligation (Moore et al., 1992, Science 256, 9923 or WO 93/23569), or by hybridization following synthesis and/or deprotection.

In yet another example, the RNAi molecule contemplated for use in the method of the disclosure is a nucleic acid e.g., a DNA molecule, from which a miRNA, pri-miRNA or pre-miRNA of the disclosure can be transcribed. Accordingly, the method of the disclosure may comprise administering to the subject a nucleic acid so that a miRNA, pri-miRNA or pre-miRNA encoded thereby can be transcribed in the subject.

In one example, the nucleic acid is DNA. Suitable DNAs from which RNAi molecules of the disclosure may be transcribed are described herein.

In one example, the nucleic acid contemplated for use in the method of the disclosure comprises a sequence encoding a pri-miRNA or pre-miRNA corresponding to a miRNA selected from the group of miRNAs set forth in Table 1 or Table 2.

In one example, the nucleic acid comprises a sequence encoding a pri-miRNA or pre-miRNA corresponding to a miRNA selected from the group of lethal miRNAs set forth in Table 1. For example, the nucleic acid may comprise a sequence encoding a pri-miRNA or pre-miRNA corresponding to a miRNA selected from the group of lethal miRNAs set forth in rows 1-20 of Table 1. For example, the nucleic acid may comprise a sequence encoding a pri-miRNA or pre-miRNA corresponding to a miR-515 family member selected from the group of lethal miRNAs set forth in Table 1. In one example, the nucleic acid comprises a sequence encoding a pri-miRNA or pre-miRNA corresponding to a lethal miRNA selected from the group of consisting of miR-516b-5p, miR-517a-3p, miR-517c-3p, miR-518b, miR-519e-5p and miR-526b-5p. In one example, the nucleic acid comprises a sequence encoding a pri-miRNA or pre-miRNA corresponding to miR-516b-5p. In one example, the nucleic acid comprises a sequence encoding a pri-miRNA or pre-miRNA corresponding to miR-517a-3p. In one example, the nucleic acid comprises a sequence encoding a pri-miRNA or pre-miRNA corresponding to miR-517c-3p. In one example, the nucleic acid comprises a sequence encoding a pri-miRNA or pre-miRNA corresponding to miR-518b. In one example, the nucleic acid comprises a sequence encoding a pri-miRNA or pre-miRNA corresponding to miR-519e-5p. In one example, the nucleic acid comprises a sequence encoding a pri-miRNA or pre-miRNA corresponding to miR-526b-5p.

In another example, the nucleic acid may comprise a sequence encoding a pri-miRNA or pre-miRNA corresponding to a miRNA selected from the group of synthetic lethal miRNAs set forth in Table 2. For example, the nucleic acid may comprise a sequence encoding a pri-miRNA or pre-miRNA corresponding to a miRNA selected from the group of synthetic lethal miRNAs set forth in rows 1-13 of Table 2. For example, the nucleic acid may comprise a sequence encoding a pri-miRNA or pre-miRNA corresponding to a miR-515 family member selected from the group of synthetic lethal miRNAs set forth in Table 2. In one example, the nucleic acid comprises a sequence encoding a pri-miRNA or pre-miRNA corresponding to a synthetic lethal miRNA selected from the group of consisting miR-380-3p, miR-99b-5p and miR-485-3p. In one example, the nucleic acid comprises a sequence encoding a pri-miRNA or pre-miRNA corresponding to miR-380-3p. In one example, the nucleic acid comprises a sequence encoding a pri-miRNA or pre-miRNA corresponding to miR-99b-5p. In one example, the nucleic acid comprises a sequence encoding a pri-miRNA or pre-miRNA corresponding to miR-485-3p.

In one example, the nucleic acid contemplated for use in the method of the disclosure comprises a sequence encoding a miRNA, pri-miRNA or pre-miRNA comprising an effector sequence of a miRNA set forth in Table 1 or Table 2.

In one example, the nucleic acid comprises a sequence encoding a miRNA, pri-miRNA or pre-miRNA comprising an effector sequence of a lethal miRNA set forth in Table 1. For example, the nucleic acid may comprise a sequence encoding a miRNA, pri-miRNA or pre-miRNA comprising an effector sequence of a lethal miRNA set forth in rows 1-20 of Table 1. For example, the nucleic acid may comprise a sequence encoding a miRNA, pri-miRNA or pre-miRNA comprising an effector sequence of a miR-515 family member which is a lethal miRNA set forth in Table 1. In one example, the nucleic acid comprises a sequence encoding a miRNA, pri-miRNA or pre-miRNA comprising an effector sequence of a lethal miRNA selected from the group of consisting of miR-516b-5p, miR-517a-3p, miR-517c-3p, miR-518b, miR-519e-5p and miR-526b-5p. In one example, the nucleic acid comprises a sequence encoding a miRNA, pri-miRNA or pre-miRNA comprising an effector sequence of miR-516b-5p. In one example, the nucleic acid comprises a sequence encoding a miRNA, pri-miRNA or pre-miRNA comprising an effector sequence of miR-517a-3p. In one example, the nucleic acid comprises a sequence encoding a miRNA, pri-miRNA or pre-miRNA comprising an effector sequence of miR-517c-3p. In one example, the nucleic acid comprises a sequence encoding a pri-miRNA or pre-miRNA comprising an effector sequence of miR-518b. In one example, the nucleic acid comprises a sequence encoding a miRNA, pri-miRNA or pre-miRNA comprising an effector sequence of miR-519e-5p. In one example, the nucleic acid comprises a sequence encoding a miRNA, pri-miRNA or pre-miRNA comprising an effector sequence of miR-526b-5p.

In one example, the nucleic acid comprises a sequence encoding a miRNA, pri-miRNA or pre-miRNA comprising an effector sequence of a synthetic lethal miRNAs set forth in Table 2. For example, the nucleic acid may comprise a sequence encoding a miRNA, pri-miRNA or pre-miRNA comprising an effector sequence of a synthetic lethal miRNAs set forth in rows 1-13 of Table 2. For example, the nucleic acid may comprise a sequence encoding a miRNA, pri-miRNA or pre-miRNA comprising an effector sequence of a miR-515 family member which is a synthetic lethal miRNA set forth in Table 2. In one example, the nucleic acid comprises a sequence encoding a miRNA, pri-miRNA or pre-miRNA comprising an effector sequence of a synthetic lethal miRNA selected from the group of consisting of miR-380-3p, miR-99b-5p and miR-485-3p. In one example, the nucleic acid comprises a sequence encoding a miRNA, pri-miRNA or pre-miRNA comprising an effector sequence of miR-380-3p. In one example, the nucleic acid comprises a sequence encoding a miRNA, pri-miRNA or pre-miRNA comprising an effector sequence of miR-99b-5p. In one example, the nucleic acid comprises a sequence encoding a miRNA, pri-miRNA or pre-miRNA comprising an effector sequence of miR-485-3p.

In one example, the nucleic acid contemplated for use in the method of the disclosure comprises a sequence encoding a miRNA, pri-miRNA or pre-miRNA comprising an effector sequence which targets the same mRNA transcript sequence as an effector sequence of a miRNA set forth in Table 1 or Table 2.

In one example, the nucleic acid comprises a sequence encoding a miRNA, pri-miRNA or pre-miRNA comprising an effector sequence which targets the same mRNA transcript sequence as an effector sequence of a lethal miRNA set forth in Table 1. For example, the nucleic acid may comprise a sequence encoding a miRNA, pri-miRNA or pre-miRNA comprising an effector sequence which targets the same mRNA transcript sequence as an effector sequence of a lethal miRNA set forth in rows 1-20 of Table 1. For example, the nucleic acid may comprise a sequence encoding a miRNA, pri-miRNA or pre-miRNA comprising an effector sequence which targets the same mRNA transcript sequence as an effector sequence of a miR-515 family member which is a lethal miRNA set forth in Table 1. In one example, the nucleic acid comprises a sequence encoding a miRNA, pri-miRNA or pre-miRNA comprising an effector sequence which targets the same mRNA transcript sequence as an effector sequence of a lethal miRNA selected from the group of consisting of miR-516b-5p, miR-517a-3p, miR-517c-3p, miR-518b, miR-519e-5p and miR-526b-5p. In one example, the nucleic acid comprises a sequence encoding a miRNA, pri-miRNA or pre-miRNA comprising an effector sequence which targets the same mRNA transcript sequence as an effector sequence of miR-516b-5p. In one example, the nucleic acid comprises a sequence encoding a miRNA, pri-miRNA or pre-miRNA comprising an effector sequence which targets the same mRNA transcript sequence as an effector sequence of miR-517a-3p. In one example, the nucleic acid comprises a sequence encoding a miRNA, pri-miRNA or pre-miRNA comprising an effector sequence which targets the same mRNA transcript sequence as an effector sequence of miR-517c-3p. In one example, the nucleic acid comprises a sequence encoding a miRNA, pri-miRNA or pre-miRNA comprising an effector sequence which targets the same mRNA transcript sequence as an effector sequence of miR-518b. In one example, the nucleic acid comprises a sequence encoding a miRNA, pri-miRNA or pre-miRNA comprising an effector sequence which targets the same mRNA transcript sequence as an effector sequence of miR-519e-5p. In one example, the nucleic acid comprises a sequence encoding a miRNA, pri-miRNA or pre-miRNA comprising an effector sequence which targets the same mRNA transcript sequence as an effector sequence of miR-526b-5p.

For example, the nucleic acid may comprise a sequence encoding a miRNA, pri-miRNA or pre-miRNA comprising an effector sequence which targets the same mRNA transcript sequence as an effector sequence of a synthetic lethal miRNA set forth in Table 2. For example, the nucleic acid may comprise a sequence encoding a miRNA, pri-miRNA or pre-miRNA comprising an effector sequence which targets the same mRNA transcript sequence as an effector sequence of a synthetic lethal miRNA set forth in rows 1-13 of Table 2. For example, the nucleic acid may comprise a sequence encoding a miRNA, pri-miRNA or pre-miRNA comprising an effector sequence which targets the same mRNA transcript sequence as an effector sequence of a miR-515 family member which is a synthetic lethal miRNA set forth in Table 2. In one example, the nucleic acid comprises a sequence encoding a miRNA, pri-miRNA or pre-miRNA comprising an effector sequence which targets the same mRNA transcript sequence as an effector sequence of a synthetic lethal miRNA selected from the group of consisting of miR-380-3p, miR-99b-5p and miR-485-3p. In one example, the nucleic acid comprises a sequence encoding a miRNA, pri-miRNA or pre-miRNA comprising an effector sequence which targets the same mRNA transcript sequence as an effector sequence of miR-380-3p. In one example, the nucleic acid comprises a sequence encoding a miRNA, pri-miRNA or pre-miRNA comprising an effector sequence which targets the same mRNA transcript sequence as an effector sequence of miR-99b-5p. In one example, the nucleic acid comprises a sequence encoding a miRNA, pri-miRNA or pre-miRNA comprising an effector sequence which targets the same mRNA transcript sequence as an effector sequence of miR-485-3p.

In some examples, a nucleic acid contemplated for use in a method of the disclosure comprises one or more additional elements e.g., to facilitate transcription of the miRNA, pri-miRNA or pre-miRNA encoded thereby. For example, the or each nucleic acid may comprise a promoter operably-linked to a sequence encoding a miRNA, pri-miRNA or pre-miRNA of the disclosure. Other elements e.g., transcriptional terminators, are known in the art and/or described herein. For example, the nucleic acid encoding the RNAi molecule of the disclosure may be linked to a suitable promoter and provided as an expression construct.

In circumstances where it is advantageous to administer more than one of the RNAi molecules of the disclosure to the subject, a nucleic acid described herein may encode a plurality of the RNAi molecules of the disclosure. In one example, the sequences of the nucleic acid encoding the respective RNAi molecules may be operably-linked to the same promoter. In another example, the sequences encoding the respective RNAi molecules of the disclosure may be operably-linked to different promoters.

Often the nucleic acid encoding the RNAi molecule of the disclosure is provided within a vector, e.g., a plasmid or a miniplasmid or a viral vector. Accordingly, vectors comprising one or more nucleic acid(s) encoding RNAi molecules of the disclosure are contemplated for use in the method of the disclosure. Suitable expression vectors for use in the method of the disclosure are described herein. Accordingly, the present disclosure contemplates the use of an expression vector comprising a nucleic acid encoding an RNAi molecule of the disclosure for treating neuroblastoma.

As discussed herein, a nucleic acid encoding an RNAi molecule of the disclosure may be linked to, or in operable linkage with, a promoter for controlling expression of the respective RNAi molecule(s).

In one example, the promoter is a constitutive promoter. The term “constitutive” when made in reference to a promoter means that the promoter is capable of directing transcription of an operably linked nucleic acid sequence in the absence of a specific stimulus (e.g., heat shock, chemicals, light, etc.). Typically, constitutive promoters are capable of directing expression of a coding sequence in substantially any cell and any tissue. The promoters used to transcribe the RNAi molecules of the disclosure include a promoter for ubiquitin, CMV, β-actin, histone H4, EF-1α or pgk genes controlled by RNA polymerase II, or promoter elements controlled by RNA polymerase I.

In one example, a Pol II promoter such as CMV, SV40, U1, β-actin or a hybrid Pol II promoter is employed.

In another example, a promoter controlled by RNA polymerase III is used, such as a U6 promoter (U6-1, U6-8, U6-9), H1 promoter, 7SL promoter, a human Y promoter (hY1, hY3, hY4 (see Maraia, et al., Nucleic Acids Res 22(15):3045-52 (1994)) and hY5 (see Maraia, et al., Nucleic Acids Res 24(18):3552-59 (1994)), a human MRP-7-2 promoter, an Adenovirus VA1 promoter, a human tRNA promoter, or a 5s ribosomal RNA promoter.

In one example, the promoter is an RNA Pol III promoter. For example, the promoter is a U6 promoter. In another example, the promoter is a H1 promoter.

In some examples, promoters of variable strength are employed. For example, use of two or more strong promoters (such as a Pol III-type promoter) may tax the cell, by, e.g., depleting the pool of available nucleotides or other cellular components needed for transcription. In addition or alternatively, use of several strong promoters may cause a toxic level of expression of RNAi molecules in the cell. Thus, in some examples where a nucleic acid encoding a plurality of RNAi molecules of the disclosure is linked to, or in operable linkage with, a plurality of promoters, one or more of those promoters may be weaker than other promoters in the respective expression construct, or all promoters in the construct may express the RNAi molecules at less than a maximum rate. Promoters also may be modified using molecular techniques, or otherwise, e.g., through regulation elements, to attain weaker levels of transcription.

Promoters useful in some examples of the present disclosure can be tissue-specific or cell-specific. The term “tissue specific” as it applies to a promoter refers to a promoter that is capable of directing selective transcription of a nucleic acid of interest to a specific type of tissue (e.g., neural crest-derived tissue) in the relative absence of expression of the same nucleotide sequence of interest in a different type of tissue (e.g., liver). The term “cell-specific” as applied to a promoter refers to a promoter which is capable of directing selective transcription of a nucleic acid of interest in a specific type of cell in the relative absence of expression of the same nucleotide sequence of interest in a different type of cell within the same tissue.

In one example, a nucleic acid of the disclosure, or expression vector comprising same, may additionally comprise one or more enhancers to increase expression of the or each RNAi molecule. Suitable enhancers will be known to those skilled in the art.

In a further example, a nucleic acid of the disclosure, or expression vector comprising same, may comprise a transcriptional terminator linked to the nucleic acid encoding an RNAi molecule of the disclosure. The terminator used will be dependent on the type of promoter employed (e.g., a Pol II- or Pol III-type promoter). Suitable terminators will be known to those skilled in the art.

In some examples, where different promoters are used, the terminators can be different and are matched to the promoter from the gene from which the terminator is derived. Such terminators include the SV40 poly A, the Ad VA1 gene, the 5S ribosomal RNA gene, and the terminators for human t-RNAs. In addition, promoters and terminators may be mixed and matched, as is commonly done with RNA pol II promoters and terminators.

In addition, the nucleic acid encoding an RNAi molecule of the disclosure, or the expression vector comprising same, can comprise one or more multiple cloning sites and/or unique restriction sites that are located strategically, such that the promoter, RNAi molecule encoding sequence and/or terminator elements are easily removed or replaced. The nucleic acid encoding an RNAi molecule of the disclosure, or the expression vector comprising same, can be assembled from smaller oligonucleotide components using strategically located restriction sites and/or complementary sticky ends. The base vector for one approach according to the present disclosure comprises plasmids with a multilinker in which all sites are unique (though this is not an absolute requirement). Sequentially, each promoter is inserted between its designated unique sites resulting in a base cassette with one or more promoters, all of which can have variable orientation. Sequentially, again, annealed primer pairs are inserted into the unique sites downstream of each of the individual promoters, resulting in a single-, double- or multiple-expression cassette construct. The insert can be moved into a suitable expression vector using two unique enzyme sites (the same or different ones) that flank the single-, double- or multiple-expression cassette insert.

Generation of nucleic acids encoding RNAi molecules useful in the method of the disclosure, or expression constructs and vectors comprising same, can be accomplished using any suitable genetic engineering techniques known in the art, including without limitation, the standard techniques of PCR, oligonucleotide synthesis, restriction endonuclease digestion, ligation, transformation, plasmid purification, and DNA sequencing. If the or each nucleic acid encoding an RNAi molecule of the disclosure is to be packaged into a viral particle for delivery to a subject, the or each nucleic acid may comprise or be linked to sequences necessary to package the nucleic acid(s) into viral particles and/or sequences that allow integration of the nucleic acid(s) into the target cell genome. In some examples, the or each nucleic acid encoding an RNAi molecule of the disclosure may additionally contain or be linked to genes that allow for replication and propagation of virus, however such genes will be supplied in trans. Additionally, the or each nucleic acid encoding an RNAi molecule of the disclosure can contain genes or genetic sequences from the genome of any known organism incorporated in native form or modified. For example, a viral construct may comprise sequences useful for replication of the construct in bacteria.

The or each nucleic acid encoding an RNAi molecule of the disclosure may also contain or be linked to additional genetic elements. The types of elements that may be included in, or linked to, the nucleic acid(s) are not limited in any way and may be chosen by one with skill in the art. For example, additional genetic elements may include a reporter gene, such as one or more genes for a fluorescent marker protein such as GFP or RFP; an easily assayed enzyme such as beta-galactosidase, luciferase, beta-glucuronidase, chloramphenical acetyl transferase or secreted embryonic alkaline phosphatase; or proteins for which immunoassays are readily available such as hormones or cytokines.

Other genetic elements that may find use in examples of the present disclosure include those coding for proteins which confer a selective growth advantage on cells such as adenosine deaminase, aminoglycodic phosphotransferase, dihydrofolate reductase, hygromycin-B-phosphotransferase, drug resistance, or those genes coding for proteins that provide a biosynthetic capability missing from an auxotroph. If a reporter gene is included along with the or each nucleic acid, an internal ribosomal entry site (IRES) sequence can be included. In one example, the additional genetic elements are operably linked with and controlled by an independent promoter/enhancer. In addition a suitable origin of replication for propagation of the nucleic acid in bacteria may be employed. The sequence of the origin of replication generally is separated from the nucleic acid encoding an RNAi molecule of the disclosure and other genetic sequences. Such origins of replication are known in the art and include the pUC, ColE1, 2-micron or SV40 origins of replication.

As discussed above, it may be advantageous for a nucleic encoding an RNAi molecule of the disclosure to be included within an expression vector. The expression vector may be administered to the subject to be treated such that that the RNAi molecule encoded by the nucleic acid is transcribed within the subject.

In one example, the expression vector is a plasmid, e.g., as is known in the art.

In one example, the expression vector is mini-circle DNA. Mini-circle DNA is described in U.S. Patent Publication No. 2004/0214329. Mini-circle DNA is useful for persistently high levels of nucleic acid transcription. The circular vectors are characterized by being devoid of expression-silencing bacterial sequences. For example, mini-circle vectors differ from bacterial plasmid vectors in that they lack an origin of replication, and lack drug selection markers commonly found in bacterial plasmids, e.g. β-lactamase, tet, and the like. Consequently, minicircle DNA becomes smaller in size, allowing more efficient delivery.

In one example, the expression vector is a viral vector.

A viral vector based on any appropriate virus may be used to deliver a nucleic acid encoding an RNAi molecule of the disclosure. In addition, hybrid viral systems may be of use. The choice of viral delivery system will depend on various parameters, such as the tissue targeted for delivery, transduction efficiency of the system, pathogenicity, immunological and toxicity concerns, and the like.

Commonly used classes of viral systems used in gene therapy can be categorized into two groups according to whether their genomes integrate into host cellular chromatin (oncoretroviruses and lentiviruses) or persist in the cell nucleus predominantly as extrachromosomal episomes (adeno-associated virus, adenoviruses and herpesviruses). In one example, a viral vector of the disclosure integrates into a host cell's chromatin. In another example, a viral vector of the disclosure persists in a host cell's nucleus as an extrachomosomal episome.

In one example, a viral vector is an adenoviral (AdV) vector. Adenoviruses are medium-sized double-stranded, non-enveloped DNA viruses with linear genomes that is between 26-48 Kbp. Adenoviruses gain entry to a target cell by receptor-mediated binding and internalization, penetrating the nucleus in both non-dividing and dividing cells. Adenoviruses are heavily reliant on the host cell for survival and replication and are able to replicate in the nucleus of vertebrate cells using the host's replication machinery.

In one example, a viral vector is from the Parvoviridae family. The Parvoviridae is a family of small single-stranded, non-enveloped DNA viruses with genomes approximately 5000 nucleotides long. Included among the family members is adeno-associated virus (AAV). In one example, a viral vector of the disclosure is an AAV. AAV is a dependent parvovirus that generally requires co-infection with another virus (typically an adenovirus or herpesvirus) to initiate and sustain a productive infectious cycle. In the absence of such a helper virus, AAV is still competent to infect or transduce a target cell by receptor-mediated binding and internalization, penetrating the nucleus in both non-dividing and dividing cells. Because progeny virus is not produced from AAV infection in the absence of helper virus, the extent of transduction is restricted only to the initial cells that are infected with the virus. It is this feature which makes AAV a desirable vector for the present disclosure. Furthermore, unlike retrovirus, adenovirus, and herpes simplex virus, AAV appears to lack human pathogenicity and toxicity (Kay, et al., Nature. 424: 251 (2003)). Since the genome normally encodes only two genes it is not surprising that, as a delivery vehicle, AAV is limited by a packaging capacity of 4.5 single stranded kilobases (kb). However, although this size restriction may limit the genes that can be delivered for replacement gene therapies, it does not adversely affect the packaging and expression of shorter sequences, such as pre-miRNAs and pri-miRNAs.

Another viral delivery system which may be useful for delivering a nucleic acid encoding an RNAi molecule of the disclosure is a system based on viruses from the family Retroviridae. Retroviruses comprise single-stranded RNA animal viruses that are characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

In some examples, a viral vector is a lentivirus. Lentivirus vectors are often pseudotyped with vesicular steatites virus glycoprotein (VSV-G), and have been derived from the human immunodeficiency virus (HIV); visan-maedi, which causes encephalitis (visna) or pneumonia in sheep; equine infectious anemia virus (EIAV), which causes autoimmune hemolytic anemia and encephalopathy in horses; feline immunodeficiency virus (FIV), which causes immune deficiency in cats; bovine immunodeficiency virus (BIV) which causes lymphadenopathy and lymphocytosis in cattle; and simian immunodeficiency virus (SIV), which causes immune deficiency and encephalopathy in non-human primates. Vectors that are based on HIV generally retain <5% of the parental genome, and <25% of the genome is incorporated into packaging constructs, which minimizes the possibility of the generation of reverting replication-competent HIV. Biosafety has been further increased by the development of self-inactivating vectors that contain deletions of the regulatory elements in the downstream long-terminal-repeat sequence, eliminating transcription of the packaging signal that is required for vector mobilization. The main advantage to the use of lentiviral vectors is that gene transfer is persistent in most tissues or cell types.

A lentiviral-based construct used to express an RNA of the disclosure comprises sequences from the 5′ and 3′ long terminal repeats (LTRs) of a lentivirus. In one example, the viral construct comprises an inactivated or self-inactivating 3′ LTR from a lentivirus. The 3′ LTR may be made self-inactivating by any method known in the art. For example, the U3 element of the 3′ LTR contains a deletion of its enhancer sequence, e.g., the TATA box, Spl and NF-kappa B sites. As a result of the self-inactivating 3′ LTR, the provirus that is integrated into the host genome will comprise an inactivated 5′ LTR. The LTR sequences may be LTR sequences from any lentivirus from any species. The lentiviral-based construct also may incorporate sequences for MMLV or MSCV, RSV or mammalian genes. In addition, the U3 sequence from the lentiviral 5′ LTR may be replaced with a promoter sequence in the viral construct. This may increase the titer of virus recovered from the packaging cell line. An enhancer sequence may also be included.

Other viral or non-viral systems known to those skilled in the art may be used to deliver the RNAi molecule of the present invention to cells of interest, including but not limited to gene-deleted adenovirus-transposon vectors (see Yant, et al., Nature Biotech. 20:999-1004 (2002)); systems derived from Sindbis virus or Semliki forest virus (see Perri, et al, J. Virol. 74(20):9802-07 (2002)); systems derived from Newcastle disease virus or Sendai virus.

### Assaying RNAi Molecules of the Disclosure

### Cell Culture Models

RNAi molecules which are suitable for use in the method of the disclosure will preferably be capable of reducing viability of a neuroblastoma cell e.g., by conferring a lethal phenotype to the cell, when administered alone or in combination with a chemotherapeutic agent. Accordingly, one or more functional assays may be performed to determine the ability of the RNAi molecule to affect viability and/or confer a lethal or synthetic lethal phenotype to neuroblastoma cells. Suitable functional assays are described in Examples 1-3 hereof.

Exemplary cell lines useful as cell culture models for neuroblastoma are the KELLY and SHEP cell lines described in Example 1.

As exemplified herein, activity of an RNAi molecule of the disclosure is determined by administering the RNAi molecule to the cell and subsequently assaying viability of the cell using a CellTiter-Glo luminescent assay. An RNAi molecule which is able to reduce neuroblastoma cell viability by at least 50% may be considered a “lethal” RNAi molecule and useful in the method of the disclosure e.g., as a standalone or combination therapy.

In addition, suitability of an RNAi molecule as an adjunctive agent for treatment of neuroblastoma may be evaluated. For example, the activity of an RNAi molecule may be determined by administering the RNAi molecule to the cell in combination with a chemotherapeutic agent e.g., a low dose IC30 of vincristine or doxorubicin. Cell viability may then be determined using a CellTiter-Glo luminescent assay. An RNAi molecule which decreases cell viability by less than 30% when administered alone, but which decreases cell viability by more than 50% when administered with a chemotherapeutic agent relative to the viability of cells which have been administered the RNAi molecule alone, is considered a “synthetic lethal” RNAi molecule and useful in the combination treatment method of the disclosure.

### Animal Models

Several small animal models are available for studying neuroblastoma. An exemplary mouse model for pre-clinical testing of RNAi molecules of the disclosure in treatment of neuroblastoma is the TH-MYCN mouse. This neuroblastoma-prone mouse recapitulates many of the features of human neurobiastoma.

Additional animal models of neuroblastoma are described and reviewed in H. Iwakura and T. Akamizu, Neuroblastoma Mouse Models, pp 31-38, In: MA Hayat (ed.) Neuroblastoma, Pediatric Cancer 1 (2012), the entire contents of which is incorporated herein by reference.

Any of the foregoing animal models can be used to determine the efficacy of an RNAi molecule of the disclosure to treat neuroblastoma and/or sensitise a neuroblastoma to a chemotherapeutic drug and/or treat neuroblastoma in combination with a chemotherapeutic drug.

### Pharmaceutical Compositions

Any one or more RNAi molecules of the disclosure, or a nucleic acid or expression vector encoding same as described herein, may be incorporated into a pharmaceutical composition. Accordingly, the present disclosure also provides a pharmaceutical composition comprising any one or more RNAi molecule, a nucleic acid or an expression vector described herein. It is contemplated that the pharmaceutical composition may be used in a method of treating neuroblastoma as described herein.

A pharmaceutical composition of the disclosure will preferably comprise a therapeutically effective amount of the RNAi molecule or a nucleic acid or expression vector encoding same, for treating neuroblastoma. As described herein, a “therapeutically effective amount” is at least the minimum concentration or amount required to effect a measurable improvement of the neuroblastoma. A therapeutically effective amount herein may vary according to factors such as the disease state, age, sex, and weight of the patient, and the ability of the RNAi molecule, nucleic acid, or expression vector to elicit a desired response in the individual. A therapeutically effective amount is also one in which any toxic or detrimental effects of the RNAi molecule, or nucleic acid or expression vector encoding same, are outweighed by the therapeutically beneficial effects.

The pharmaceutical composition of the disclosure may also comprise one or more pharmaceutically-acceptable carriers, excipients or diluents. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent may be selected with regard to the intended route of administration and standard pharmaceutical practice. For example, the carrier included in the pharmaceutical composition may be suited for delivery of the RNAi molecule, nucleic acid or expression vector of the disclosure to neural crest-derived tissue of a subject following administration of the composition thereto.

In one example, an RNAi molecule described herein may be formulated for delivery using the “EnGeneIC Delivery Vehicle” (EDV) system developed by EnGeneIC Molecular Delivery Pty Ltd (Sydney), which is based on the use of intact, bacterially derived minicells. The EDV™ system is described, for example, in published international applications WO2006/021894 and WO2009/027830, the respective contents of which are incorporated here by reference. Thus, one example of a pharmaceutical composition of the disclosure may comprise the one or more RNAi molecules, nucleic acids or expression vectors as described herein loaded into EDVs i.e., intact minicells.

Minicells which are useful for delivery of the RNAi molecules of the disclosure and which may be provided in the pharmaceutical compositions described herein are typically anucleate forms of E. coli or other bacterial cells, engendered by a disturbance in the coordination, during binary fission, of cell division with DNA segregation. Prokaryotic chromosomal replication is linked to normal binary fission, which involves mid-cell septum formation. In E. coli, for example, mutation of min genes, such as minCD, can remove the inhibition of septum formation at the cell poles during cell division, resulting in production of a normal daughter cell and an anucleate minicell. Minicells are distinct from other small vesicles that are generated and released spontaneously in certain situations and, in contrast to minicells, are not due to specific genetic rearrangements or episomal gene expression. In the present disclosure, it is desirable for minicells to have intact cell walls (“intact minicells”).

In addition to min operon mutations, anucleate minicells also are generated following a range of other genetic rearrangements or mutations that affect septum formation, for example in the JMVB 1 in B. subtilis. Minicells also can be formed following a perturbation in the levels of gene expression of proteins involved in cell division/chromosome segregation. For example, overexpression of minE leads to polar division and production of minicells. Similarly, chromosome-less minicells may result from defects in chromosome segregation for example the smc mutation in Bacillus subtilis, spoOJ deletion in B. subtilis, mukB mutation in E. coli, and parC mutation in E. coli. Gene products may be supplied in trans. When over-expressed from a high-copy number plasmid, for example, CafA may enhance the rate of cell division and/or inhibit chromosome partitioning after replication, resulting in formation of chained cells and anucleate minicells. Minicells can be prepared from any bacterial cell of Gram-positive or Gram-negative origin.

The RNAi molecules, nucleic acid and expression vectors described herein may therefore be packaged directly into intact minicells. This process bypasses the previously required steps of, for example, cloning nucleic acids encoding functional nucleic acid into expression plasmids, transforming minicell-producing parent bacteria with the plasmids and generating recombinant minicells. Instead, plasmid-free nucleic acid molecules can be packaged directly into intact minicells by co-incubating a plurality of intact minicells with RNAi molecules, nucleic acids or expression vectors described herein in a buffer. In some embodiments, the co-incubation may involve gentle shaking, while in others the co-incubation is static. A co-incubation period of about one hour has proven sufficient, but shorter periods, such as about half an hour, also may be effective. In one example, the buffer comprises buffered saline, for example an 1× phosphate buffer solution. The buffered saline can be in gelatin form. In another example, the co-incubation is conducted at a temperature of about 4° C. to about 37° C.; about 20° C. to about 30° C.; about 25° C.; or about 37° C. In other examples, the co-incubation can comprise about 10⁷, 10⁸, 10⁹, 10¹⁰, 1θ″, 10¹² or 10¹³ minicells. Specific parameters of temperature, time, buffer, minicell concentration, etc. can be optimized for a particular combination of conditions.

Once packaged, the RNAi molecules, nucleic acid and expression vectors of the disclosure remain inside the minicell and are protected from degradation. In this regard, prolonged incubation studies with siRNA-packaged minicells incubated in sterile saline showed no leakage of siRNAs. In addition, co-incubating siRNA-packaged minicells with nucleases confirmed that the siRNAs had penetrated the outer membrane of the intact minicells and were protected from degradation. Similarly, despite the fact that minicells might be expected to carry residual nucleases from the parent bacterial cytoplasm, packaged siRNA are stable in the minicell cytoplasm. Packaged siRNA also avoid the degradative machinery present within phagolysosomes, such as acids, free oxygen radicals and acid hydrolases (Conner and Schmid, 2003), to effect target mRNA knockdown within the mammalian cell. Thus, this approach is particularly useful with the RNAi molecules of the disclosure.

It is also contemplated that multiple RNAi molecules, nucleic acid and/or expression vectors described herein directed to different mRNA targets can be packaged in the same minicell. Such an approach can be used to combat drug resistance and apoptosis resistance. For example, cancer patients routinely exhibit resistance to chemotherapeutic drugs. Such resistance can be mediated by over-expression of genes such as multi-drug resistance (MDR) pumps and anti-apoptotic genes, among others. To combat this resistance, minicells can be packaged with therapeutically significant concentrations of RNAi molecules, nucleic acid and expression vectors of the disclosure and administered to a patient before, during or after chemotherapy. Furthermore, packaging into the same minicell multiple RNAi molecules, nucleic acid and expression vectors described herein directed to different mRNA targets can enhance therapeutic success since most molecular targets are subject to mutations and have multiple alleles.

In some examples, the carrier is a lipid-based carrier, cationic lipid, or liposome nucleic acid complex, a liposome, a micelle, a virosome, a lipid nanoparticle or a mixture thereof.

In some examples, the carrier is a biodegradable polymer-based carrier, such that a cationic polymer-nucleic acid complex is formed. For example, the carrier may be a cationic polymer microparticle suitable for delivery of an RNAi molecule, nucleic acid or expression vector of the disclosure to a neuroblastoma cell. Use of cationic polymers for delivery compositions to cells is known in the art, such as described in Judge et al. Nature 25: 457-462 (2005), the contents of which is incorporated herein by reference. An exemplary cationic polymer-based carrier is a cationic DNA binding polymer, such as polyethylenimine. Other cationic polymers suitable for complexing with, and delivery of, RNAi molecules, nucleic acids and expression vectors of the disclosure include poly(L-lysine) (PLL), chitosan, PAMAM dendrimers. and poly(2-dimethylamino)ethyl methacrylate (pDMAEMA). These are other suitable cationic polymers are known in the art and are described in Mastrobattista and Hennink, Nature Materials, 11:10-12 (2012), WO/2003/097107 and WO/2006/041617, the full contents of which are incorporated herein by reference. Such carrier formulations have been developed for various delivery routes including parenteral subcutaneous injection, intravenous injection and inhalation.

In a further example, the carrier is DOTAP and/or some other cationic lipid-mediated nucleic acid delivery system. DOTAP has been used for the systemic delivery of a siRNA for gene slicing, e.g., Sorenson et al, J. Mol. Biol. 327:761-766 (2003), the entire contents of which is incorporated herein by reference.

In a further example, the carrier is a cyclodextrin-based carrier such as a cyclodextrin polymer-nucleic acid complex.

In a further example, the carrier is a protein-based carrier such as a cationic peptide-nucleic acid complex.

In another example, the carrier is a lipid nanoparticle. Exemplary nanoparticles are described, for example, in U.S. Pat. No. 7,514,099.

In some examples, an RNAi molecule, nucleic acid or expression vector of the disclosure is formulated with a lipid nanoparticle composition comprising a cationic lipid/Cholesterol/PEG-C-DMA/DSPC (e.g., in a 40/48/2/10 ratio), a cationic lipid/Cholesterol/PEG-DMG/DSPC (e.g., in a 40/48/2/10 ratio), or a cationic lipid/Cholesterol/PEG-DMG (e.g., in a 60/38/2 ratio). In some examples, the cationic lipid is Octyl CL in DMA, DL in DMA, L-278, DLinKC2DMA, or MC3.

In another example, an RNAi molecule, nucleic acid or expression vector of the disclosure is formulated with any of the cationic lipid formulations described in WO 2010/021865; WO 2010/080724; WO 2010/042877; WO 2010/105209 or WO 2011/022460.

In another example, an RNAi molecule, nucleic acid or expression vector of the disclosure is conjugated to or complexed with another compound, e.g., to facilitate delivery of the RNAi molecule, nucleic acid or expression vector. Non-limiting, examples of such conjugates are described in US 2008/0152661 and US 2004/0162260 (e.g., CDM-LBA, CDM-Pip-LBA, CDM-PEG, CDM-NAG, etc.).

In another example, polyethylene glycol (PEG) is covalently attached to an RNAi molecule, nucleic acid or expression vector of the disclosure. The attached PEG can be any molecular weight, e.g., from about 100 to about 50,000 daltons (Da).

In yet other example, an RNAi molecule, nucleic acid or expression vector of the disclosure is formulated with a carrier comprising surface-modified liposomes containing poly(ethylene glycol) lipids (PEG-modified, or long-circulating liposomes or stealth liposomes), such as is disclosed in for example, WO 96/10391; WO 96/10390; or WO 96/10392.

In some examples, an RNAi molecule, nucleic acid or expression vector of the disclosure can also be formulated or complexed with polyethyleneimine or a derivative thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives. In one example, an RNAi molecule, nucleic acid or expression vector of the disclosure is formulated as described in U.S. Patent Application Publication No. 2003/0077829.

In other examples, an RNAi molecule, nucleic acid or expression vector of the disclosure is complexed with membrane disruptive agents such as those described in U.S. Patent Application Publication No. 2001/0007666.

Other carriers include cyclodextrins (see for example, Gonzalez et al., 1999, Bioconjugate Chem., 10, 1068-1074; or WO 03/46185), poly(lactic-co-glycolic)acid (PLGA) and PLCA microspheres (see for example US 2002130430).

As described herein, certain RNAi molecules of the disclosure have been shown to be particularly useful in treating neuroblastoma when administered in combination with a chemotherapeutic agent e.g., at low dose IC30. It therefore follows that a pharmaceutical composition of the disclosure may comprise, or be packaged together with, one or more chemotherapeutic agents approved for treatment of neuroblastoma. Accordingly, the present disclosure provides a pharmaceutical composition comprising an RNAi molecule, nucleic acid or expression vector of the disclosure and a chemotherapeutic agent, either formulated together or provided as separate components for administration together. In one example, the RNAi molecule, nucleic acid or expression vector which is formulated or packaged together with the chemotherapeutic agent corresponds to a lethal RNAi molecule described herein e.g., as described in Table 1. In another example, the RNAi molecule, nucleic acid or expression vector which is formulated or packaged together with the chemotherapeutic agent corresponds to a synthetic lethal RNAi molecule described herein e.g., as described in Table 2. As described herein, synthetic lethal RNAi molecules of the disclosure are those that have been shown reduce neuroblastoma cell viability when administered in combination with a chemotherapeutic agent.

The chemotherapeutic agent may be selected from a vinca alkaloid, a taxane, a platinum-based agent, an anthracycline, an alkylating agent, and/or a topoisomerase inhibitor. In one example, the chemotherapeutic agent is a vinca alkaloid e.g., vincristine, vinblastine, vinorelbine or vindesine. In one example, the chemotherapeutic agent is a taxane e.g., paclitaxel, docetaxel and cabazitaxel. In one example, the chemotherapeutic agent is a platinum-based agent e.g., cisplatin, carboplatin, oxaliplatin or nedaplatin. In one example, the chemotherapeutic agent is an anthracycline e.g., daunorubicin, doxorubicin, epirubicin, idarubicin or mitoxantrone. In one example, the chemotherapeutic agent is an alkylating agent e.g., cyclophosphamide, melphalan or dacarbazine. In one example, the chemotherapeutic agent is a topoisomerase inhibitor e.g., etoposide, a camptothecin, or a camptothecins derivative (such as topotecan). Other suitable chemotherapeutic drugs for treatment of neuroblastoma will be known in the art are are contemplated herein e.g., as described in Pinto et al. (2015), Journal of Clinical Oncology, 33(27):3008-3017.

A pharmaceutical composition of the disclosure may desirably include materials that increase the biological stability of the RNAi molecule, nucleic acid or expression vector of the disclosure and/or materials that increase the ability of the compositions to localise to neuroblastoma cells and/or target neuroblastoma cells selectively. The pharmaceutical compositions of the disclosure may be administered in pharmaceutically acceptable carriers (e.g., physiological saline), which are selected on the basis of the mode and route of administration, and standard pharmaceutical practice. One having ordinary skill in the art can readily formulate a pharmaceutical composition that comprises an RNAi molecule, nucleic acid or expression vector of the disclosure. In some cases, an isotonic formulation is used. Generally, additives for isotonicity can include sodium chloride, dextrose, mannitol, sorbitol and lactose. In some cases, isotonic solutions such as phosphate buffered saline are preferred. Stabilizers include gelatin and albumin. In some examples, a vasoconstriction agent is added to the formulation. The compositions according to the present disclosure are provided sterile and pyrogen free. Suitable pharmaceutical carriers, as well as pharmaceutical necessities for use in pharmaceutical formulations, are described in Remington: The Science and Practice of Pharmacy (formerly Remington's Pharmaceutical Sciences), Mack Publishing Co., a standard reference text in this field, and in the USP/NF.

Pharmaceutical compositions of the disclosure can also comprise conventional pharmaceutical excipients and/or additives. Suitable pharmaceutical excipients include stabilizers, antioxidants, osmolality adjusting agents, buffers, and pH adjusting agents. Suitable additives include, e.g., physiologically biocompatible buffers (e.g., tromethamine hydrochloride), additions of chelants (e.g., DTPA or DTPA-bisamide) or calcium chelate complexes (e.g., calcium DTPA, CaNaDTPA-bisamide), or, optionally, additions of calcium or sodium salts (e.g., calcium chloride, calcium ascorbate, calcium gluconate or calcium lactate).

The pharmaceutical composition of the disclosure may be conveniently formulated according to the desired route od administration to a subject. Routes of administration include, but are not limited to, intramuscular, intraperitoneal, intradermal, subcutaneous, intravenous, intraarterially, intraoccularly and oral as well as transdermal or by inhalation or suppository. Exemplary routes of administration include intravenous, intramuscular, oral, intraperitoneal, intradermal, intraarterial and subcutaneous injection. In one example, the pharmaceutical composition of the disclosure is formulated for intravenous administration.

Such compositions are useful for pharmaceutical applications and may readily be formulated in a suitable sterile, non-pyrogenic vehicle, e.g., buffered saline for injection, for parenteral administration e.g., intravenously (including intravenous infusion), IM, SC, and for intraperitoneal administration.

The volume, concentration, and formulation of the pharmaceutical composition as well as the dosage regimen may be tailored specifically to maximize cellular delivery while minimizing toxicity such as an inflammatory response e.g, relatively large volumes (5, 10, 20, 50 ml or more) with corresponding low concentrations of active ingredients, as well as the inclusion of an anti-inflammatory compound such as a corticosteroid, may be utilized if desired.

### Methods of Administration

As described herein, an RNAi molecule, nucleic acid, expression vector or composition of the disclosure may be used to treat neuroblastoma, either as a standalone treatment or as an adjunct to chemotherapy. Thus, the present disclosure provides methods for treating neuroblastoma by administering to a subject suffering from neuroblastoma an RNAi molecule, a nucleic acid, an expression vector or a pharmaceutical composition of the disclosure.

The neuroblastoma to be treated may be refractory to treatment with the chemotherapeutic agent e.g., in the absence of adjunctive treatment with the RNAi molecule, a nucleic acid or an expression vector of the disclosure. Alternatively, or in addition, the neuroblastoma to be treated may be a recurrent neuroblastoma.

The RNAi molecules, nucleic acids, expression vectors or pharmaceutical compositions of the disclosure can be administered to a subject by any means suitable for delivery of the RNAi molecule(s), nucleic acid(s) or expression vector(s) to neuroblastoma cells of the subject. For example, when the RNAi molecules of the disclosure is a miRNA or a corresponding precursor molecule or miRNA mimic as described herein, the RNAi molecule can be administered by any methods suitable to transfect cells of the subject with the RNAi molecule. Similarly, when the RNAi molecule is a nucleic acid encoding a miRNA or corresponding precursor molecule of the disclosure, the nucleic acid or expression vector comprising same may be transfected or transduced into the cells of a subject. For example, a subject to be treated may be transfected with a plasmid, or transduced with viral vector, comprising one or more nucleic acids encoding one or more RNAi molecules of the disclosure.

To circumvent the problems associated with inefficient delivery in vivo, an RNAi molecule described herein e.g., miRNA or miRNA mimic, may be delivered via the “EnGeneIC Delivery Vehicle” system developed by EnGeneIC Molecular Delivery Pty Ltd (Sydney), which is based on the use of intact, bacterially derived minicells. The EDV™ system is described, for example, in published international applications WO2006/021894 and WO2009/027830, the respective contents of which are incorporated here by reference.

Thus, in one example, the RNAi molecule(s) described here are delivered using intact, bacterially derived minicells. These minicells are delivered specifically to target tissues, using bispecific antibodies. One arm of such an antibody has specificity for the target tissue, while the other has specificity for the minicell. The antibody brings minicells to the target cell surface, and then the minicells are brought into the cell by endocytosis. After uptake into the tumour cell there is a release of the minicell contents, i.e., the RNAi molecule of the disclosure. For an antibody in this regard, specificity against any cell surface marker for neuroblastoma e.g., GD2, could be used in accordance with the disclosure.

Other methods of administering nucleic acids are well known in the art. In particular, the routes of administration already in use for nucleic acid therapeutics, along with formulations in current use, provide preferred routes of administration and formulation for the RNAi molecules, nucleic acids, expression vectors and compositions of the disclosure. For example, the RNAi molecules, nucleic acids, expression vectors and compositions described herein can be administered by a number of routes including, but not limited to intramuscular, intraperitoneal, intradermal, subcutaneous, intravenous, intraarterially, intraoccularly, intrapleural, and oral as well as transdermal or by inhalation or suppository.

The RNAi molecules, nucleic acids, and expression vectors of the disclosure can also be administered via liposomes or nanoparticles i.e., which are present in a composition of the disclosure. Such administration routes and appropriate formulations are described herein and generally known to those of skill in the art. Administration of the formulations described herein may be accomplished by any acceptable method that allows the RNAi molecule, nucleic acid or expression vector of the disclosure to reach its target. The particular mode selected will depend of course, upon exemplary factors such as the particular formulation, the severity of the state of the subject being treated, and the dosage required for therapeutic efficacy.

Other delivery systems suitable include but are not limited to time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these.

### Dosing

Dosages for treating a particular subject for neuroblastoma can be determined by one of ordinary skill in the art using conventional considerations, (e.g., by means of an appropriate, conventional pharmacological protocol). A physician may, for example, prescribe a relatively low dose at first, subsequently increasing the dose until an appropriate response is obtained. The dose administered to a subject is sufficient to effect a beneficial therapeutic response in the subject over time, or, e.g., to reduce symptoms, or other appropriate activity, depending on the application. The dose is determined by the efficacy of the particular formulation, and the activity, stability or serum half-life of the RNAi molecule employed and the condition of the subject, as well as the body weight or surface area of the subject to be treated.

Preferably, a dosage of the RNAi molecule, nucleic acid, expression vector or composition comprising same for use in the method of the disclosure will be sufficient to reduce viability of a neuroblastoma cell in the subject e.g., by at least 50%.

The dosage suitable for use in the method of the disclosure is preferably one that avoids or minimises undue adverse side effects. Adverse side effects may include but are not limited to nausea, vomiting, toxicity, irritation, allergic response, and death. An adverse side effects may be “undue” when the risk outweighs the benefit provided by an RNAi molecule described herein.

The size of the dose is also determined by the existence, nature, and extent of any adverse side-effects that accompany the administration of a particular vector, and formulation, in a particular subject.

### Combination Therapy

As described herein, certain RNAi molecules of the disclosure have been shown to be particularly useful in treating neuroblastoma when administered in combination with a chemotherapeutic agent e.g., at low dose IC30. It therefore follows that the RNAi molecule, nucleic acid, expression vector or composition of the disclosure may be used as an adjunct to conventional chemotherapy approved for treatment of neuroblastoma. It is contemplated that synthetic lethal RNAi molecules of the disclosure e.g., such as those related to the miRNAs in Table 2 herein, may be particularly suited for combination therapy, as these RNAi molecules were identified based on the criteria that they reduce neuroblastoma cell viability in the presence of a chemotherapeutic agent, but not on their own. Naturally, it will be appreciated by a person skilled on the art that lethal RNAi molecules of the disclosure e.g., such as those related to the miRNAs in Table 1 herein, may also be efficacious in combination therapy.

In one example, an RNAi molecule, nucleic acid, expression vector or composition described herein may be administered to a subject suffering from neuroblastoma in order to reduce a therapeutically effective dose of a chemotherapeutic agent which is effective for treating the neuroblastoma i.e., relative to a dose of the chemotherapeutic agent which is therapeutically effective in a subject who has not or will not be administered an RNAi molecule, nucleic acid, expression vector or composition of the disclosure. For example, the reduced dose of the chemotherapeutic agent may be a IC30 concentration of the chemotherapeutic agent. In another example, the reduced dose of the chemotherapeutic agent may be a IC10 concentration of the chemotherapeutic agent. Methods for determining efficacy of a combination therapy described herein will be known to a person skilled in the art.

Alternatively, or in addition, an RNAi molecule, nucleic acid, expression vector or composition described herein may be used to improve efficacy of a chemotherapeutic agent to treat neuroblastoma in a subject suffering therefrom i.e., relative to the efficacy of the chemotherapeutic agent to treat neuroblastoma in a subject who has not or will not be administered an RNAi molecule, nucleic acid, expression vector or composition of the disclosure. For example, an RNAi molecule, nucleic acid, expression vector or composition of the disclosure may be used to improve efficacy of a chemotherapeutic agent to treat neuroblastoma in a subject who would otherwise be refractory to treatment with the chemotherapeutic agent in the absence of receiving the RNAi molecule, nucleic acid, expression vector or composition of the disclosure. For example, an RNAi molecule, nucleic acid, expression vector or composition of the disclosure may be used to improve efficacy of a chemotherapeutic agent to treat neuroblastoma in a subject who has previously entered remission and relapsed i.e., suffering from recurrent neuroblastoma.

In one example, the combination therapy method of the disclosure comprises administering an RNAi molecule, nucleic acid, expression vector or composition described herein to a subject with neuroblastoma who has previously received chemotherapy. Alternatively, or in addition, the RNAi molecule, nucleic acid, expression vector or composition described herein is used as a pre-treatment for a subject suffering from neuroblastoma and is administered prior to administration of a chemotherapeutic agent. The administration of an RNAi molecule, nucleic acid, expression vector or composition described herein may therefore precede and/or follow administration of a chemotherapeutic agent e.g., by intervals ranging from minutes to weeks. In another example, an RNAi molecule, nucleic acid, expression vector or composition described herein is used as an adjunctive treatment for a patient suffering from neuroblastoma and is administered at substantially the same time as a chemotherapeutic agent or together.

In each of the foregoing examples, the combination therapy method may further comprise actively administering to the subject the chemotherapeutic agent. In one example, the chemotherapeutic agent and the RNAi molecule, nucleic acid, expression vector or composition described herein are administered together e.g., in the same composition. In one example, the chemotherapeutic agent and the RNAi molecule, nucleic acid, expression vector or composition described herein are administered concurrently e.g., as separate compositions. In one example, the chemotherapeutic agent and the RNAi molecule, nucleic acid, expression vector or composition described herein are administered sequentially.

According to an example where the chemotherapeutic agent and the RNAi molecule, nucleic acid, expression vector or composition described herein are administered separately to the subject, one would generally ensure that a significant period of time did not expire between the time of each delivery, such that the chemotherapeutic agent and the RNAi molecule, nucleic acid, expression vector or composition would still be able to exert an advantageously combined effect on the neuroblastoma cell. This would be particularly important for combination therapy involving RNAi molecules, nucleic acid, expression vectors and compositions which are or which express or contain “synthetic lethal” miRNAs or precursors or mimics thereof i.e., those that are therapeutically effective in combination with a chemotherapeutic agent only.

The chemotherapeutic agent may be selected from a vinca alkaloid, a taxane, a platinum-based agent, an anthracycline, an alkylating agent, and/or a topoisomerase inhibitor. In one example, the chemotherapeutic agent is a vinca alkaloid e.g., vincristine, vinblastine, vinorelbine or vindesine. In one example, the chemotherapeutic agent is a taxane e.g., paclitaxel, docetaxel and cabazitaxel. In one example, the chemotherapeutic agent is a platinum-based agent e.g., cisplatin, carboplatin, oxaliplatin or nedaplatin. In one example, the chemotherapeutic agent is an anthracycline e.g., daunorubicin, doxorubicin, epirubicin, idarubicin or mitoxantrone. In one example, the chemotherapeutic agent is an alkylating agent e.g., cyclophosphamide, melphalan or dacarbazine. In one example, the chemotherapeutic agent is a topoisomerase inhibitor e.g., etoposide, a camptothecin, or a camptothecins derivative (such as topotecan). Other suitable chemotherapeutic drugs for treatment of neuroblastoma will be known in the art e.g., as described in Pinto et al. (2015), Journal of Clinical Oncology, 33(27):3008-3017.

One skilled in the art would be able, by routine experimentation, to determine an effective, non-toxic amount of an RNAi molecule, nucleic acid, expression vector or composition of the disclosure which would be required to treat a subject suffering from neuroblastoma in combination therapy. The therapeutically effective dose level for any particular patient will depend upon a variety of factors including: the type and severity of neuroblastoma to be treated; activity or dose of any adjunctive agents e.g., the chemotherapeutic agent; the composition employed; the age, body weight, general health, sex and diet of the patient; the time of administration; the route of administration; the rate of sequestration of the RNAi molecule, nucleic acid, expression vector or composition of the disclosure; the duration of the treatment; the type of chemotherapeutic drugs used in combination or coincidental with the treatment, together with other related factors well known in medicine.

### Kits

The present disclosure also provides an RNAi molecule, nucleic acid, expression vector or composition of the disclosure in a kit. The kit may comprise one or more containers. The kit typically contains an RNAi molecule, nucleic acid, expression vector or composition of the disclosure with instructions for its administration e.g., in a method of the disclosure. In some examples, the kit contains a first container comprising an RNAi molecule, nucleic acid, expression vector or composition of the disclosure and second container comprising chemotherapeutic agent, such as a vinca alkaloid, a taxane, a platinum-based agent, an anthracycline, an alkylating agent, and/or a topoisomerase inhibitor. In one example, the chemotherapeutic agent is a vinca alkaloid e.g., vincristine, vinblastine, vinorelbine or vindesine. In one example, the chemotherapeutic agent is a taxane e.g., paclitaxel, docetaxel and cabazitaxel. In one example, the chemotherapeutic agent is a platinum-based agent e.g., cisplatin, carboplatin, oxaliplatin or nedaplatin. In one example, the chemotherapeutic agent is an anthracycline e.g., daunorubicin, doxorubicin, epirubicin, idarubicin or mitoxantrone. In one example, the chemotherapeutic agent is an alkylating agent e.g., cyclophosphamide, melphalan or dacarbazine. In one example, the chemotherapeutic agent is a topoisomerase inhibitor e.g., etoposide, a camptothecin, or a camptothecins derivative (such as topotecan). In one example, the kit is intended for treatment of neuroblastoma in accordance with a method of treatment described herein.

### EXAMPLES

### Example 1—Functional Screening of miRNAs in Cancer Cells

To systematically identify miRNAs that control the survival of cancer cells, functional miRNA screens were performed using 1200 miRNAs from the miRIDIAN miRNA inhibitor and mimic Libraries (version 16.0; Dharmacon) in breast cancer, prostate cancer and neuroblastoma cancer cell lines. In doing so, the inventors identified miRNAs which, when overexpressed or administered as a single treatment, are lethal to neuroblastoma cells (hereinafter referred to as ‘lethal’ miRNAs).

### Methods

Breast cancer cell lines (MDA-MB-231, MDA-MB-468, SK-BR-3; BT474) and prostate cancer cell lines (PC3, DU145) were obtained from American Type Culture Collection (ATCC). Neuroblastoma cell lines (KELLY, SHEP) were received from Children's Cancer Institute (CCIA). The KELLY cells were selected as a MYCN amplified neuroblastoma cell line and the SHEP cells were selected as a MYCN wild-type neuroblastoma cell line.

All breast and prostate cancer cell lines were cultivated in RPMI media supplemented with 10% FCS, HEPES, and insulin. The neuroblastoma cell lines, KELLY, SHEP, were grown in 10% FCS/RPMI and 10% FCS/DMEM, respectively.

Each of the cell lines were reverse transfected in 384-well plate format with 25 nM of a miRNA from the Dharmacon miRNA mimic or inhibitor libraries (version 16.0) using liquid-handling robot Calliper Sciclone ALH3000 according to the conditions described in Table 3. After 24 hours, media was changed using BioTek 406, and 144 hours post-transfection, cell viability was determined using CellTiter-Glo luminescent assay (Promega) at 1:2 dilution. Luminescence measurements were taken on the Synergy H4 high-throughput multimode microplate reader. All screens were performed in duplicates.

TABLE 3
Transfection conditions across a panel of cell lines

| | | | | | Volume | |
|---|---|---|---|---|---|---|
| | | | | Transfection | Transfection | |
| Cell line | Cell type | Subtype | Media | Reagent | Reagent (ml) | Cells/well |
| MDA-MB-231 | Breast cancer | TNBC | RPMI supplemented with 10% FCS, | Dharmafect 4 | 0.05 | 1100 |
| | | | HEPES & insulin | | | |
| MDA-MB-168 | Breast cancer | TNBC | RPMI supplemented with 10% FCS, | Dharmafect 4 | 0.05 | 1200 |
| | | | HEPES & insulin | | | |
| SK-BR-3 | Breast cancer | HER2- | RPMI supplemented with 10% FCS, | Dharmafect 4 | 0.05 | 1500 |
| | | | HEPES & insulin | | | |
| BT474 | Breast cancer | HER2-ER- | RPMI supplemented with 10% FCS, | Dharmafect 4 | 0.03 | 1500 |
| | | | HEPES & insulin | | | |
| SHEP | Neuroblastoma | | DMEM supplemented with 10% FCS | Dharmafect 1 | 0.05 | 800 |
| KELLY | Neuroblastoma | MYCN Amp | RPMI supplemented with 10% FCS | Dharmafect 1 | 0.03 | 1100 |
| PC3 | Prostate cancer | Castrate | RPMI supplemented with 10% FCS, | Dharmafect 1 | 0.05 | 900 |
| | | resistant | HEPES & insulin | | | |
| DU145 | Prostate cancer | Castrate | RPMI supplemented with 10% FCS, | Dharmafect 1 | 0.03 | 700 |
| | | resistant | HEPES & insulin | | | |
| IMR-90 | Normal lung | | MEM supplemented with 10% FCS, | Dharmafect 1 | 0.008 | 200 |
| | | | NEAA & sodium pyruvate | | | |
| MCF-10A | Transformed | | DMEM-F12 supplemented with 5% HS, | Dharmafect 3 | 0.04 | 600 |
| | breast | | EGF, Cholera Toxin, & | | | |
| | | | Hydrocortisone Insulin | | | |

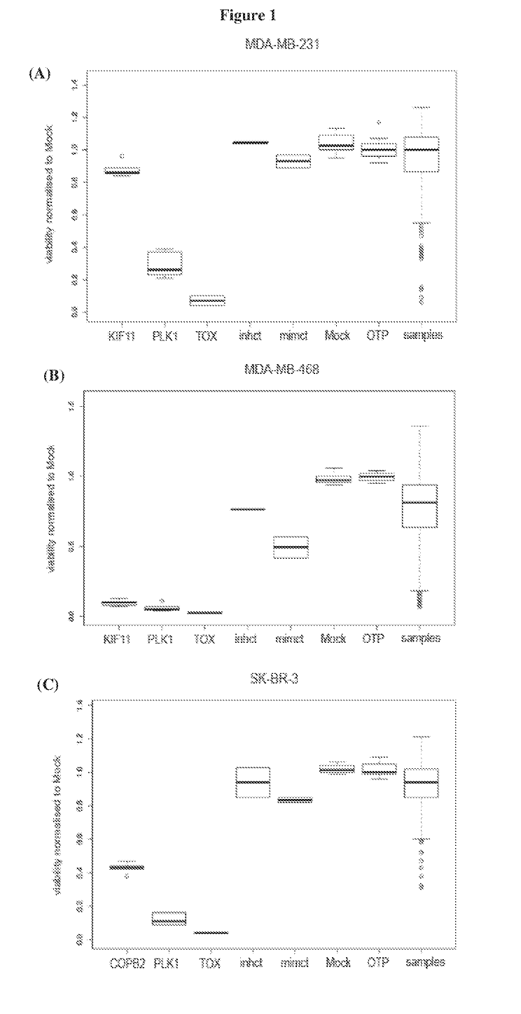

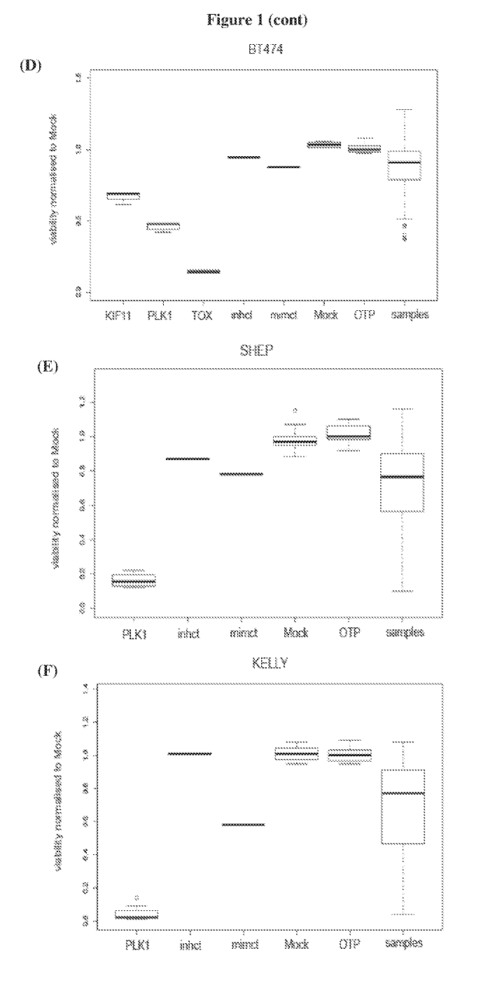

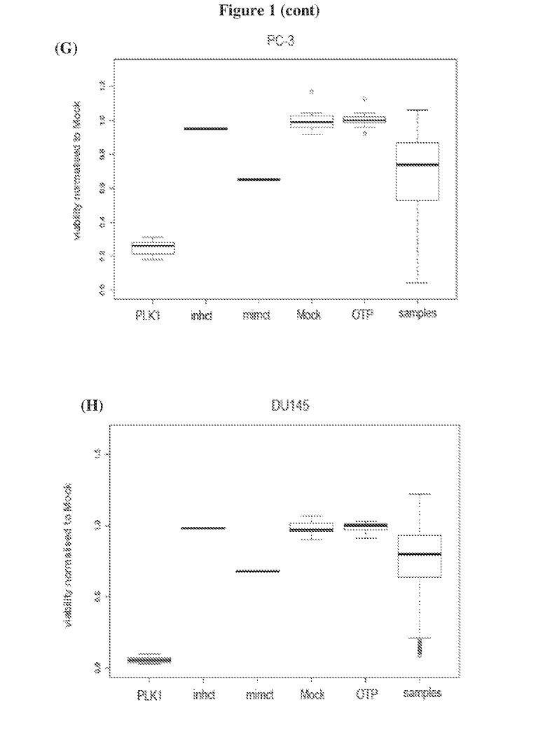

Raw viability was normalised to a nontargeting control (OTP) or mock wells, and death genes or controls inducing varying levels of death were used to monitor transfection efficiency and to define dynamic range in each screen (FIG. 1). To define screen hits, normalised viability scores were binned in the following manner: CV1 bin included candidates with viability >=0.8; CV2 bin included candidates with viability <0.8 and >0.5, and LC bin was assigned to candidates with normalised viability <=0.5. Only LC candidates were defined as screen hits.

### Results

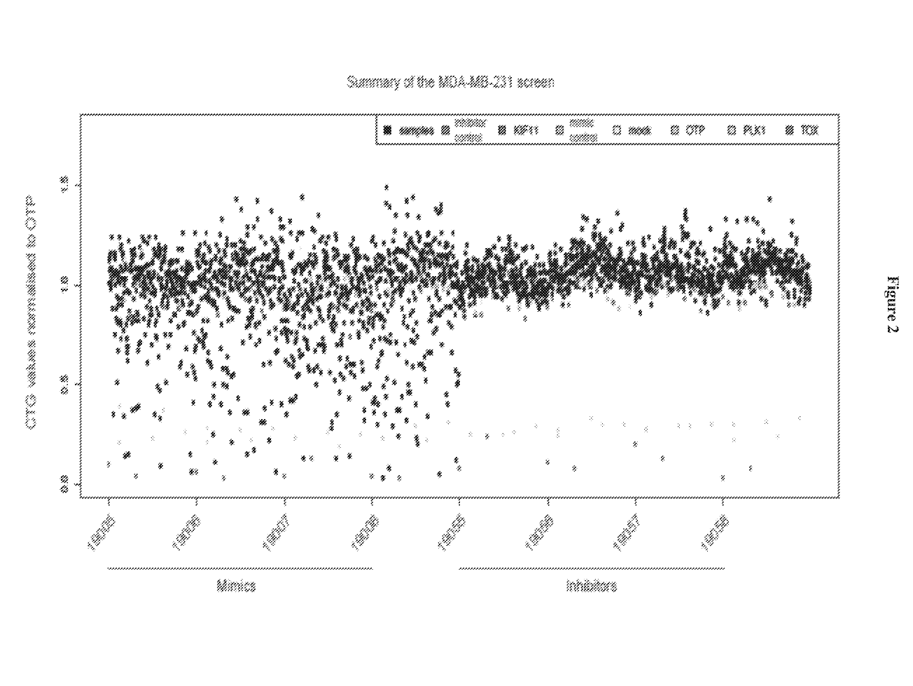

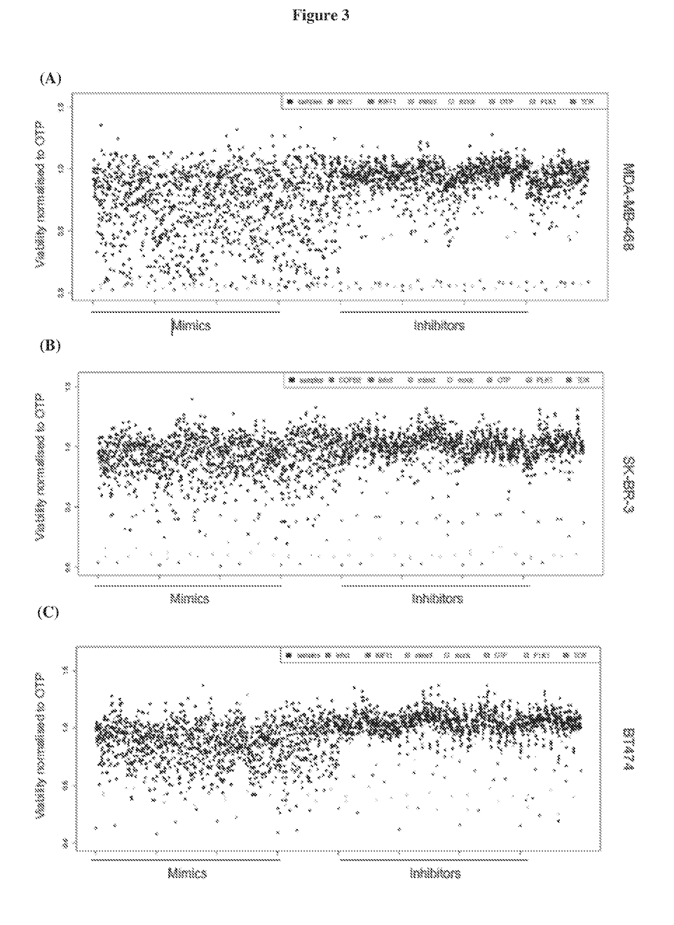

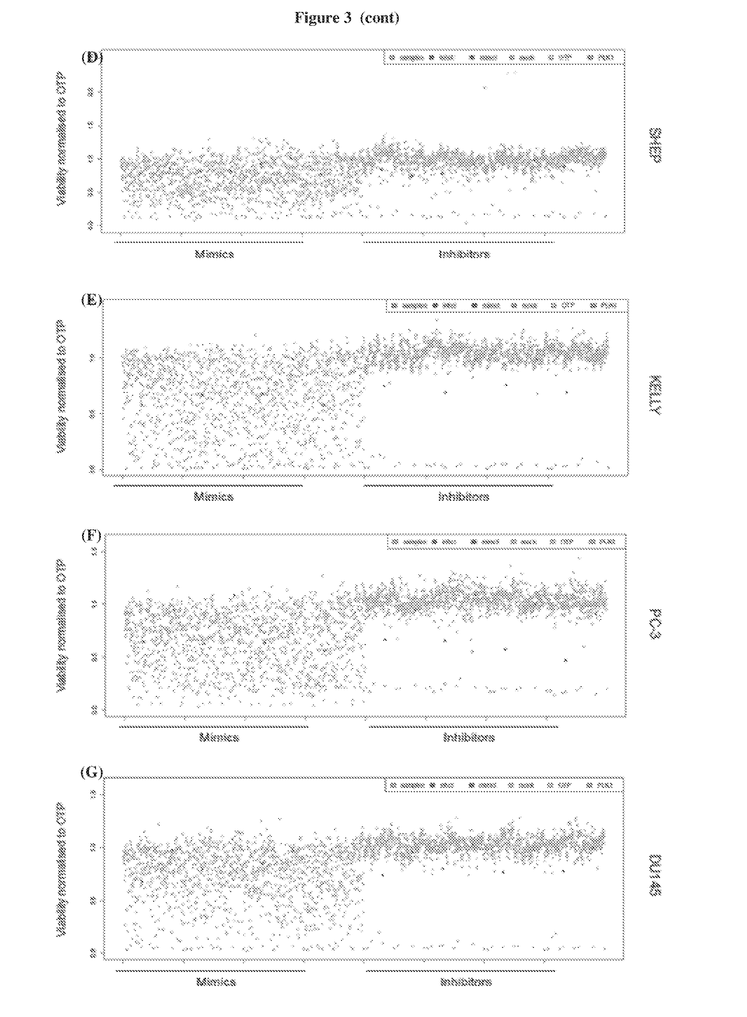

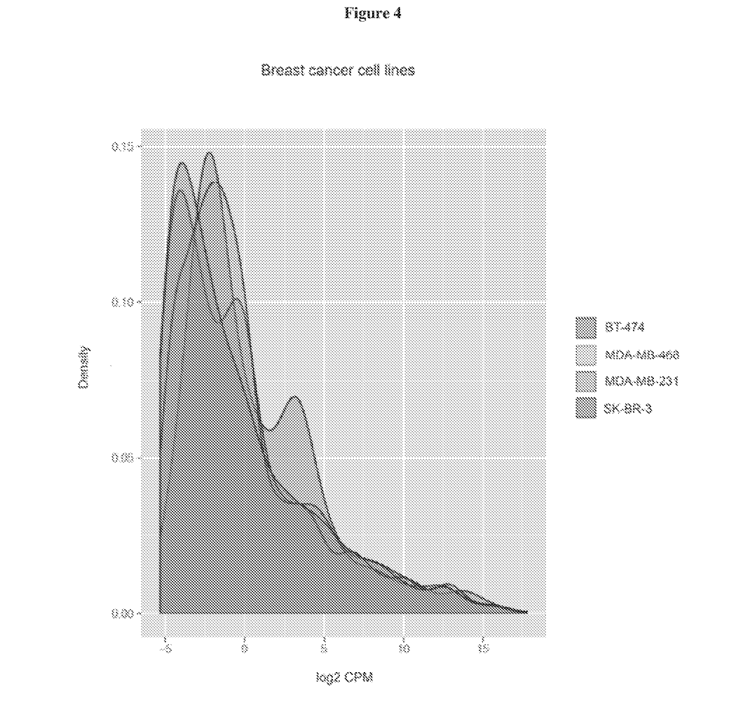

A representative graph of screen results in the MDA-MB-231 breast cancer cell line is shown in FIG. 2. Over 500 microRNA mimics or inhibitors decreased cellular viability by more than 50% in at least one cell line compared to the non-targeting control demonstrating that around 40% of microRNAs in the screen are able to regulate survival-associated pathways in cancer cells. Interestingly, a global overview of the screen results reveals profound differences in the effects of microRNA mimics and inhibitors-mimics displayed a potent killing effect across different cancers while only 10 inhibitors reduced viability more than 50% in at least one cell line (FIG. 2 and FIG. 3). This may be partially explained by the low expression levels of the majority of endogenous microRNAs in cancer cell lines where the addition of inhibitors would not be expected to lead to phenotypically observable effects (FIG. 4). Differences in miR mimic or inhibitor activities were also apparent between different cancer types and subtypes of the same cancer, with neuroblastoma cells exhibiting the highest number of disease-specific microRNAs (Table 4).

TABLE 4
Summary of disease-specific miRNAs
All primary screen hits - normalized viability <0.5

| | | | | Disease-specific |
|---|---|---|---|---|
| | | | | mimics or |
| Cell line | Mimics | Inhibitors | Cancer type | inhibitors |
| MDA-MB-231 | 103 | 0 | Breast | 48 |
| MDA-MB-468 | 329 | 0 | Neuroblastima | 103 |
| SK-BR-3 | 37 | 3 | Prostate | 49 |
| BT474 | 44 | 0 | | |
| SHEP | 286 | 5 | | |
| KELLY | 377 | 0 | | |
| PC3 | 362 | 0 | | |
| DU145 | 235 | 0 | | |

The genome-wide functional screen revealed 219 lethal miRNAs (Table 1), 140 of which were lethal when administered to or overexpressed in KELLY cells (viability <25% of scrambled control). Several of the miRNAs which were lethal in KELLY cells were also found to be lethal in SHEP non-MYCN amplified neuroblastoma cells. Those miRNAs that were found to be lethal in both KELLY and SHEP cells, and those miRNAs that were found to be potently lethal in KELLY cells specifically, were taken forward in a secondary validation screen.

Representative graphs from the primary screen illustrating the effect of microRNA mimics and inhibitors on viability of KELLY and SHEP cells are provided in FIG. 3. As is apparent, a high number of miR mimics were able to decrease cellular viability by more than 50%, whereas miRNA inhibitors had little effect on cellular viability and were not investigated further.

### Example 2—Functional Screening of miRNAs in Combination with Chemotherapeutic Agents in Neuroblastoma Cells

To systematically identify miRNAs that affect viability and survival of neuroblstoma cells in combination with chemotherapy, a further functional miRNA screen was performed using the 1200 miRNAs from the miRIDIAN miRNA mimic Library (version 16.0; Dharmacon) in combination with low dose IC30 of vincristine or doxorubicin in the two neuroblastoma cancer cell lines. In doing so, the inventors identified miRNAs which result in a lethal phenotype to neuroblastoma cells when administered in combination with low dose IC30 chemotherapy (hereinafter referred to as ‘synthetic lethal’ miRNAs).

### Methods

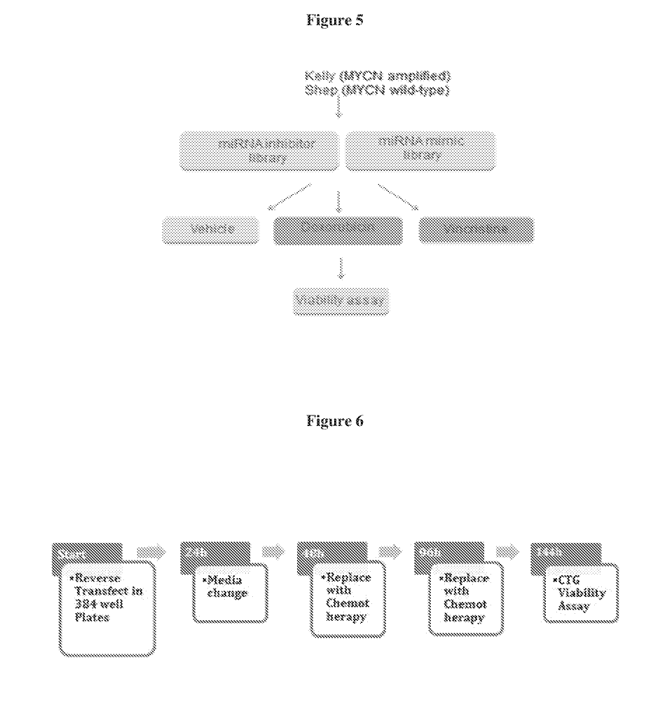

KELLY cells were grown in 10% FCS/RPMI as described in Example 1 and reverse transfected in 384-well plate format with 25 nM of a miRNA from the Dharmacon microRNA mimic Library (version 16.0) using liquid-handling robot Calliper Sciclone ALH3000 according to the conditions described in Table 3. After 24 hours, media was changed using a BioTek 406. Media was again changed after 48 hours and 96 hours using a BioTek 406, and at each of those time points low dose IC30 of vincristine or doxorubicin was added to the culture. 144 hours post-transfection, cell viability was determined using CellTiter-Glo luminescent assay (Promega) at 1:2 dilution. Luminescence measurements were taken on the Synergy H4 high-throughput multimode microplate reader as described in Example 1. All screens were performed in duplicates. The experimental design and timeline for this screen are illustrated in FIGS. 5 and 6, respectively.

miRNAs which had little or no effect upon cell viability when overexpressed as a single treatment, but which resulted in lethal phenotype when administered in combination with low dose chemotherapy, were identified (‘synthetic lethal’ miRNAs—Table 2). One group of synthetic lethal miRNAs was identified as being those miRNAs that:
(i) decreased cell viability by less than 30% when administered alone; and
(ii) when administered in combination with chemotherapy, decreased cell viability by more than 50% relative to the viability of cells at (i).

These criteria permitted identification of those miRNAs which exhibited no effect on cell viability as single-agent, but which resulted a significant lethal phenotype when administered in combination with chemotherapy. These criteria also permitted identification of those miRNAs which exhibited a negligible effect on cell viability as single-agent but a very strong lethal phenotype when administered in combination with chemotherapy.

### Results

miRNA identified as being synthetic lethal miRNAs are presented in Table 2. Seventeen satisfing the criteria (i) and (ii) above are included in Table 2. A further five miRNAs in Table 2 were identified on the basis that they decreased cell viability by more than 30% when administered alone, but were catagorised as being synthetic lethals as they showed particularly strong lethality when administered in combination with chemotherapy. These 22 sytnhetic lethal miRNAs were taken forward into the secondary validation screens.

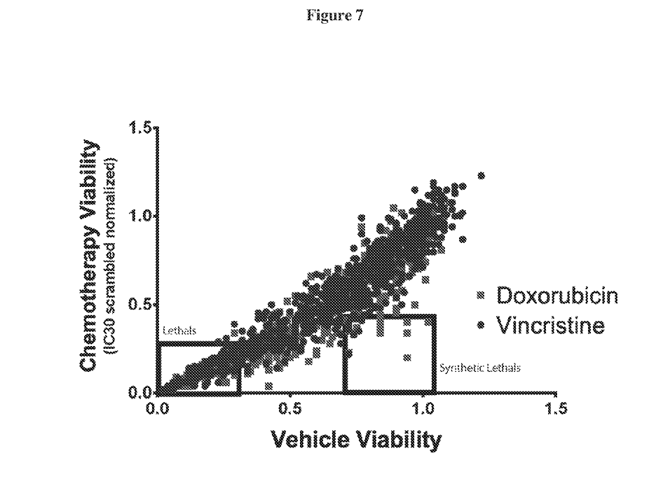

Cell viability following overexpression of each miRNA with and without low dose IC30 treatment with vincristine or doxorubicin (normalised to treatment with matched scrambled controls) is illustrated in FIG. 7.

### Example 3—Validation of Lethal and Synthetic Lethal miRNAs in Neuroblastoma Cells

The lethal miRNAs identified in Example 1 and the synthetic lethal miRNAs identified in Example 2 were taken forward into secondary validation screens to demonstrate reproducibility of the assays performed in Examples 1 and 2 respectively.

### Methods

The secondary validation screens were performed in accordance with the genome-wide screens described in Example 1 and Example 2, respectively, with the exception that an additional MYCN-amplified neuroblastoma cell line (SK-N-DZ) was added to each screen.

Furthermore, five additional non-active miRNAs discovered to have no measureable effect across all treatment groups of the genome-wide screen (‘non-lethal’ miRNAs) were included as negative controls.

### Results

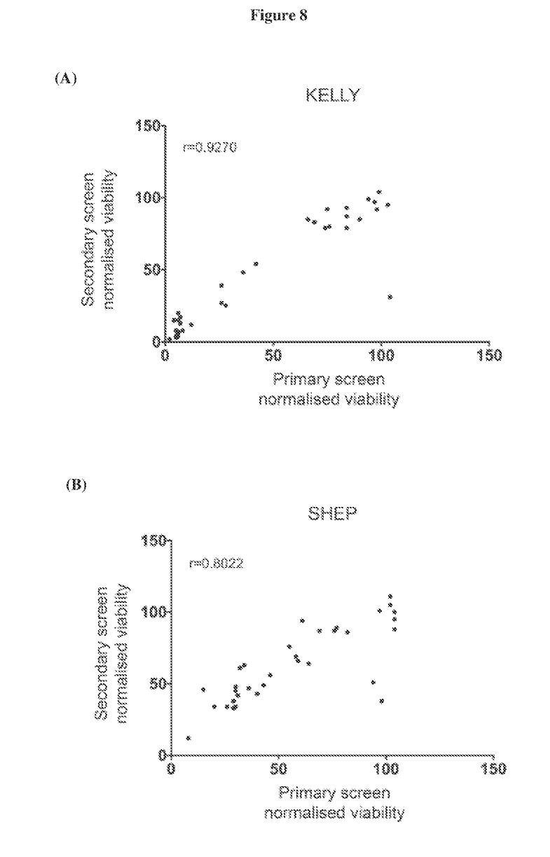

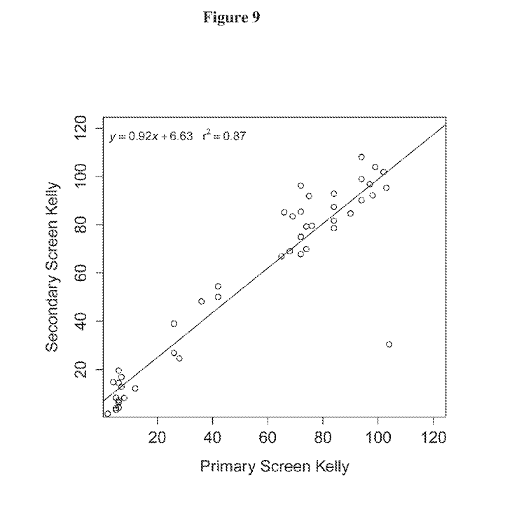

Validation of results between the primary and secondary screens demonstrated the reproducibility of the assay. A number of the lethal miRNAs identified in the primary screen were successfully validated in KELLY and SHEP cells (FIG. 8) and 13 of the synthetic lethal miRNAs were successfully validated in KELLY cells (FIG. 9). Approximately half of the miRNAs validated in KELLY cells were further validated in the additional SK-N-DZ MYCN-amplified cell line (Tables 5 and 6).

TABLE 5
Validated Lethal miRNAs (cell viability expressed as a percent
of control).

| | | | | | Additional |
|---|---|---|---|---|---|
| | Primary screen | | Secondary screen | | cell line |
| | Viability | | Viability | | Viability |
| | (% of control) | | (% of control) | | (% of control) |
| miRNA.Mimic | KELLY | SHEP | KELLY | SHEP | SKNDZ |
| hsa-miR-101-3p | 6 | 58 | 7 | 69 | 72 |
| hsa-miR-103a-2-5p | 26 | 46 | 39 | 56 | 52 |
| hsa-miR-1231 | 36 | 15 | 48 | 46 | 98 |
| hsa-miR-1262 | 8 | 31 | 8 | 42 | 13 |
| hsa-miR-128b | 6 | 30 | 4 | 48 | 22 |
| hsa-miR-148b-5p | 5 | 20 | 8 | 34 | 21 |
| hsa-miR-1911-3p | 104 | 98 | 31 | 38 | 58 |
| hsa-miR-192-3p | 42 | 43 | 54 | 49 | 97 |
| hsa-miR-196-1-5p | 6 | 29 | 15 | 33 | 53 |
| hsa-miR-489 | 5 | 40 | 4 | 43 | 33 |
| hsa-miR-491-3p | 12 | 26 | 12 | 34 | 47 |
| hsa-miR-517a-3p | 28 | — | 25 | 73 | 27 |
| hsa-miR-517c-3p | 26 | 40 | 27 | 43 | 21 |
| hsa-miR-542-3p | 6 | 29 | 20 | 38 | 28 |
| hsa-miR-544a | 7 | 32 | 17 | 61 | 59 |
| hsa-miR-550a-3p | 4 | 61 | 15 | 94 | 82 |
| hsa-miR-552 | 2 | 36 | 2 | 47 | 36 |
| hsa-miR-634 | 7 | 8 | 13 | 12 | 41 |
| hsa-miR-654-5p | 6 | 30 | 7 | 45 | 28 |
| hsa-miR-876-3p | 6 | 94 | 6 | 51 | 48 |
| hsa-miR-892b | 5 | 30 | 3 | 34 | 54 |

TABLE 6
Validated Synthetic Lethal miRNAs (cell viability expressed as a percent of
control).

| | Primary screen | | | Secondary screen | | | Additional cell line | | |
|---|---|---|---|---|---|---|---|---|---|
| | Viability (% of control) | | | Viability (% of control) | | | Viability (% of control) | | |
| | Kelly | Kelly | Kelly | Kelly | Kelly | Kelly | SKNDZ | SKNDZ | SKNDZ |
| miRNA Mimic | Vehicle | DXR | VCR | Vehicle | DXR | VCR | Vehicle | DXR | VCR |
| hsa-miR-1321 | 42 | 4 | 18 | 50 | 7 | 24 | 64 | 21 | 45 |
| hsa-miR-380-3p | 94 | 20 | 63 | 90 | 38 | 76 | 100 | 30 | 80 |
| hsa-miR-996-5p | 65 | 28 | 49 | 67 | 29 | 44 | 93 | 36 | 61 |
| hsa-miR-515-3p | 84 | 38 | 56 | 82 | 42 | 73 | 85 | 24 | 70 |
| hsa-miR-485-3p | 72 | 23 | 52 | 85 | 47 | 53 | 113 | 69 | 68 |
| hsa-miR-3181 | 94 | 34 | 57 | 108 | 61 | 96 | 91 | 38 | 83 |
| hsa-miR-3924 | 72 | 35 | 40 | 75 | 46 | 57 | 85 | 41 | 81 |
| hsa-miR-100-5p | 68 | 34 | 48 | 69 | 43 | 43 | 98 | 48 | 69 |
| hsa-miR-3151 | 102 | 40 | 52 | 102 | 65 | 47 | 87 | 71 | 72 |
| hsa-miR-3646 | 74 | 34 | 55 | 70 | 45 | 72 | 101 | 52 | 110 |
| hsa-miR-34a-5p | 72 | 70 | 36 | 96 | 98 | 35 | 92 | 74 | 57 |
| hsa-miR-449a | 72 | 62 | 22 | 75 | 83 | 35 | 71 | 49 | 44 |
| hsa-miR-34c-5p | 72 | 74 | 36 | 68 | 80 | 18 | 53 | 34 | 31 |

DXR = doxorubucin
VCR = vincristine

Of the 13 synthetic lethal miRNAs successfully validated in the secondary screen, miRNA mimics hsa-miR-1321, hsa-miR-380-3p, hsa-miR-99b-5p, hsa-miR-515-3p, hsa-miR-485-3p, and hsa-miR-34a-5p stood out as having a substantial synthetic lethality with chemotherapy in both Kelly and SKNDZ MYCN-amplified cells (note that miR-34c-5p and miR-449a have an identical seed sequence to miR-34a).

### Example 4—Analysis of Toxicity of Synthetic Lethal miRNAs in Non-Cancer Cells

The inventors sought to determine whether miRNAs identified in the functional screens are toxic to non-cancer cells.

### Methods

The cell toxicity screens were performed in IMR-90 cells (normal human fibroblast cells, ATCC). Briefly, the IMR-90 cells were cultured in MEM media supplemented with 10% FCS, NEAA and sodium pyruvate and miRNA screens were performed using hsa-miR-1321, hsa-miR-380-3p, hsa-miR-99b-5p, hsa-miR-515-3p, hsa-miR-485-3p, and hsa-miR-34a-5p, with and without low dose IC30 chemotherapy, in accordance with the methods described previously in Examples 1-3.

### Results

This toxicity screen showed that miR-380-3p, 485-3p and 99b-5p have minimal activity in IMR90 cells, even when administered in combination with a chemotherapeutic agent (Table 7).

TABLE 7
Synthetic lethal miRs in non-cancer cells (cell viability
expressed as a percent of control)

| | Media | DXR | VCR |
|---|---|---|---|
| miRNA | (% of control) | (% of control) | (% of control) |
| miR-1321 | 48 | 23 | 23 |
| miR-485-3p | 79 | 76 | 80 |
| miR-380-3p | 99 | 96 | 117 |
| miR-99b-5p | 87 | 72 | 75 |
| miR-515-3p | 59 | 50 | 51 |
| miR-34a-5p | 46 | 53 | 63 |

### Example 5—in Silico Prediction Tool for miRNA and miRNA Targets Involved in Survival of Cancer Cells

### Database and Target Prediction Tool

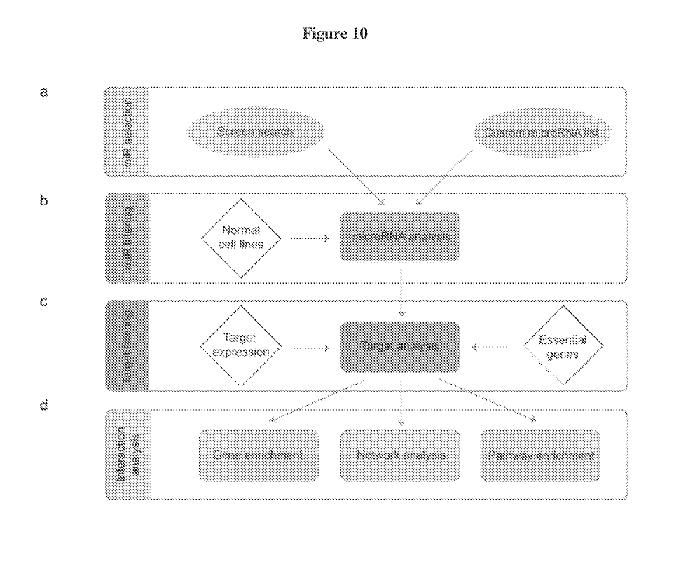

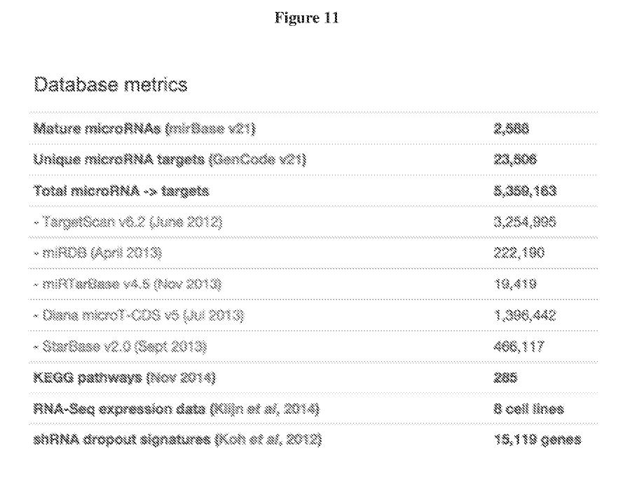

Predicting targets for a single miRNA can be a challenging task because each miRNA targets hundreds of genes simultaneously. However, as the functional screen performed in Example 1 uncovered large numbers of miRNAs inducing the same cellular phenotype of reduced viability, it was predicted that these would have overlapping targets, allowing the ranking and prioritisation of targets through integrated analysis. The inventors therefore produced a database and miRNA-target prediction tool (Moonlitt) by combining data from the primary functional genomics screens performed in Example 1 with established miRNA target prediction algorithms and pathway enrichment tools available in the public domain (FIG. 10 and FIG. 11). This database and miRNA-target prediction tool was then used to streamline the search for essential microRNAs and the mechanisms they use to suppress viability of neuroblastoma cancer cells.

As it is important to consider effects on noncancerous tissues and resultant toxicity in patients when searching for potential novel cancer therapy targets, an additional screen was performed in the non-transformed cell line MCF10A using the methodology described in Example 1 using the conditions described for MCF10A described in Table 3, and the resulting data was incorporated in the database with a published dataset generated in cardiomyocytes (described in Eulalio et al., (2012) Nature 492(7429):376-381) to identify miRNAs active only in cancer cells (FIG. 10a).

The database and miRNA-target prediction tool was designed with the ability to select which miR-target prediction algorithms are used for any particular query and what threshold(s) is/are to be applied in each case (FIG. 10b). Several widely used computational algorithms, including TargetScan, DIANA, and MiRDB, were incorporated. Importantly, already validated microRNA-mRNA interactions from high-throughput HITS-CLIP and CLASH experiments stored in StarBase and MirTarBase databases were included.

Although high-throughput methods for experimental target identification greatly improve microRNA target prediction, they are typically performed in one or few cell lines. Extreme context dependency of microRNA actions, however, limits their broader use and requires additional cell- or tissue-specific filters. To account for this, the database and miRNA-target prediction tool included RNA sequencing data for both coding and non-coding targets in each of the cell lines screened in Example 1 (i.e., MDA-MB-231, MDA-MB-468, SK-BR-3, BT474, PC3, DU145, KELLY and SHEP cells), allowing for the possibility to remove targets that are expressed below a specified threshold (FIG. 10c).

To increase the confidence of finding targets that mediate miRNA tumour-suppressor activity, the results of large siRNA essential screens in different cancer types from the DPSC-Cancer database (Koh et al., (2010) Nucleic Acids Research, 40:D957-963) were also incorporated (FIG. 10c).

The resulting database provided data on the requirement for cellular survival of over 16,000 genes in different cancer cells and was capable of determining the level of tumour-suppressor activity for each predicted miRNA target. As more data is released, it may be added to the existing database to complement the current panel of cell lines.

Since miRNAs have been shown to target multiple components of the same pathway or functional module, the database and miRNA-target prediction tool also integrated several pathway enrichment tools (FIG. 10d). KEGG analysis maps targets to pathways and produces enrichment scores for each pathway. Importantly, if a cell line was selected for expression filtering, KEGG pathway analysis will use only genes with expression (reads per kilobase mapped; RPKM) higher than specified by the user.

Validation of Database and miRNA-Target Prediction Tool

To demonstrate the validity of the inventors' database and miRNA-target prediction tool, a screen was performed using miRNAs previously shown to act as tumour suppressors in neuroblastoma through targeting MYCN. For this purpose, the data arising from the functional genomic screen performed in KELLY cells (which carries MYCN amplification) in Example 1 was queried, and miRNAs that killed over 80% of cells compared to non-targeting control were selected.

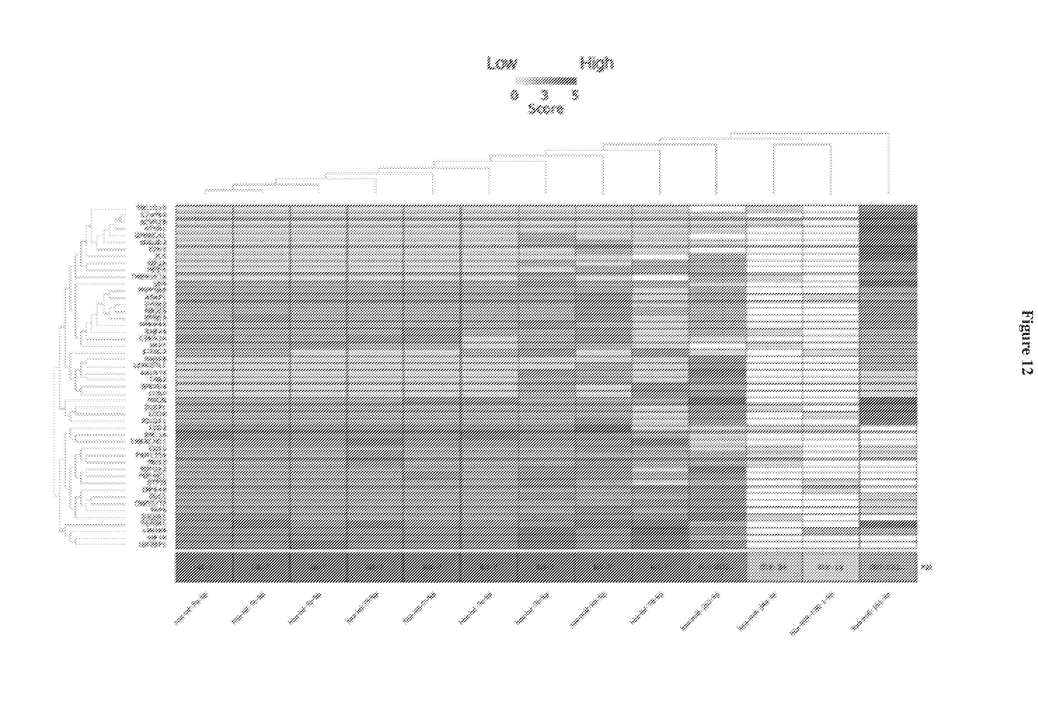

The search identified 13 miRNA (Table 8), the majority of which are already known to regulate MYCN directly (as described in Buechner et al.), including 9 members of the let-7 family, miR-19b-1-5p, miR-34a-3p, and miR-101-3p (FIG. 12).

Next, the most commonly shared targets between the miRNAs in Table 8 were identified and filtered according to their expression levels in KELLY cells (RPKM>1 for protein coding genes). Importantly, MYCN was one of the top targets, agreeing with previously published data (Table 9).

TABLE 8
miRNA targeting MYCN

| microRNA | Family |
|---|---|
| let-7a-5p | let-7 |
| let-7b-5p | let-7 |
| let-7c-5p | let-7 |
| let-7d-5p | let-7 |
| let-7e-5p | let-7 |
| let-7f-5p | let-7 |
| let-7g-5p | let-7 |
| let-7h-5p | let-7 |
| miR-98-5p | let-7 |
| miR-202-3p | let-7 |
| miR-34a-3p | miR-34 |
| miR-101-3p | miR-101 |
| miR-19b-5p | miR-19 |

TABLE 9
miRNA targets in KELLY cells

| Target | Score | # miRNAs |
|---|---|---|
| FZD3 | 1.29 | 12/13 |
| LIN28B | 1.29 | 12/13 |
| GALNT2 | 1.18 | 11/13 |
| MYCN | 1.18 | 11/13 |
| ZNF664 | 1.18 | 11/13 |
| TGFBR1 | 1.18 | 11/13 |
| ATXN1 | 1.18 | 11/13 |
| DICER1 | 1.18 | 11/13 |
| GTF21 | 1.18 | 11/13 |
| LBR | 1.18 | 11/13 |

In addition, several other bona fide oncogenes in neuroblastoma appeared among the top targets. LIN28B is a well-known target of let-7 family of microRNAs, and has been recently shown to play a major role in neuroblastoma development through a complex feedback loop involving let-7 miRs and MYCN (Molenaar et al., (2012) Nature Genetics, 44:1199-1206). IGF2BP1, another top candidate identified, is highly expressed in neuroblastoma, frequently associated with MYCN amplification, and shown to strongly promote MYCN expression levels (Bell et al., (2015) Journal of Clinical Oncology, 33:1285-1293).

### Example 6

To check whether MYCN, LIN28B and IGF2BP1 as identified in Example 5 are truly microRNA targets, KELLY cells were transfected with let-7b-5p, miR-101-3p, and miR-202-3p mimics and real-time PCR performed to determine any changes in mRNA expression levels.

Briefly, KELLY cells were grown in 10% FCS/RPMI and transfected with 25 nM of let-7b-5p, miR-101-3p, or miR-202-3p miRNA mimic using Dharmafect 1 (DF1) transfection reagent. 96 h after transfection, the cells were harvested and total RNA was extracted using miRneasy kit (Qiagen). RNA was then subjected to reverse transcription to generate cDNA. Quantification of gene expression was then performed using qPCR and Taqman probes and primers for the selected genes. The levels of gene expression were normalised to the housekeeping genes B2M and GAPDH.

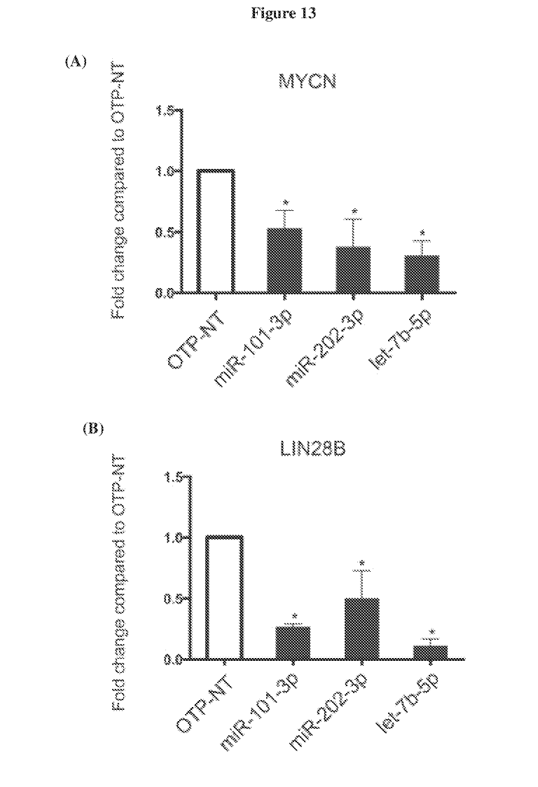

Both MYCN and LIN28B mRNA levels were strongly reduced, as predicted by the database and miRNA-target prediction tool described in Example 5 (FIG. 13). Interestingly, IGF2BP1 was not expressed in KELLY cell line precluding its further analysis.

### Example 7—Identification of Novel Factors Essential for Neuroblastoma Development

Further screens were performed for MYCN-targeting microRNAs and additional miRNA-mRNA interactions that might play a significant role in neuroblastoma development (normalised viability <=0.3).

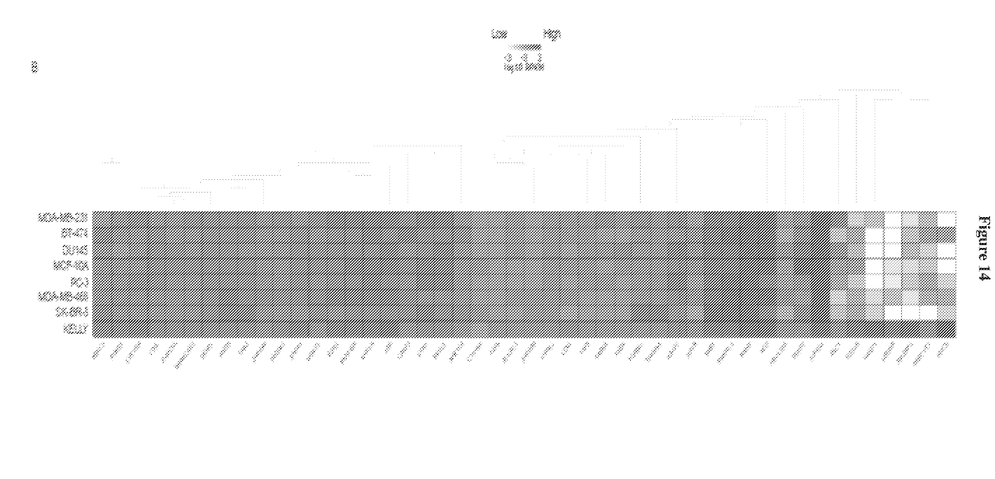

Using the Moonlitt prediction tool described in Example 5, a list of targets were identified and ranked according to the strength of predictions. The first 100 targets were enriched for two KEGG pathways, “MicroRNAs in Cancer” and “Signaling Pathways Regulating Pluripotency of Stem Cells” suggesting there might be additional targets beyond MYCN and LIN28B involved in the maintenance of poorly differentiated neuroblastoma cells. Indeed, when the targets were clustered according to their expression level across the cell line panel, a number of targets in addition to MYCN and LIN28B with high expression in KELLY cell line, but very low expression in breast and prostate cancer, were apparent (FIG. 14). Four of the targets identified which had not previously been investigated in the context of neuroblastoma, and that exhibited high expression levels in other neuroblastoma cell lines (Oncomine), were chosen for further analysis.

KELLY cells were transfected with let-7b-5p, miR-101-3p, and miR-202-3p mimics and real-time PCR performed to determine any changes in mRNA expression levels of HAND1, RGS16, RBFOX2, and ACVR2B. Briefly, KELLY cells were grown in 10% FCS/RPMI and transfected with 25 nM of let-7b-5p, miR-101-3p or miR-202-3p mimic using Dharmafect 1 (DF1) transfection reagent. 96 h after transfection, the cells were harvested and total RNA was extracted using miRneasy kit (Qiagen). RNA was then subjected to reverse transcription to generate cDNA. Quantification of gene expression was then performed using qPCR and Taqman probes and primers for HAND1, RGS 16, RBFOX2, and ACVR2B. The levels of gene expression for HAND1, RGS16, RBFOX2, and ACVR2B were normalised to the housekeeping genes B2M and GAPDH

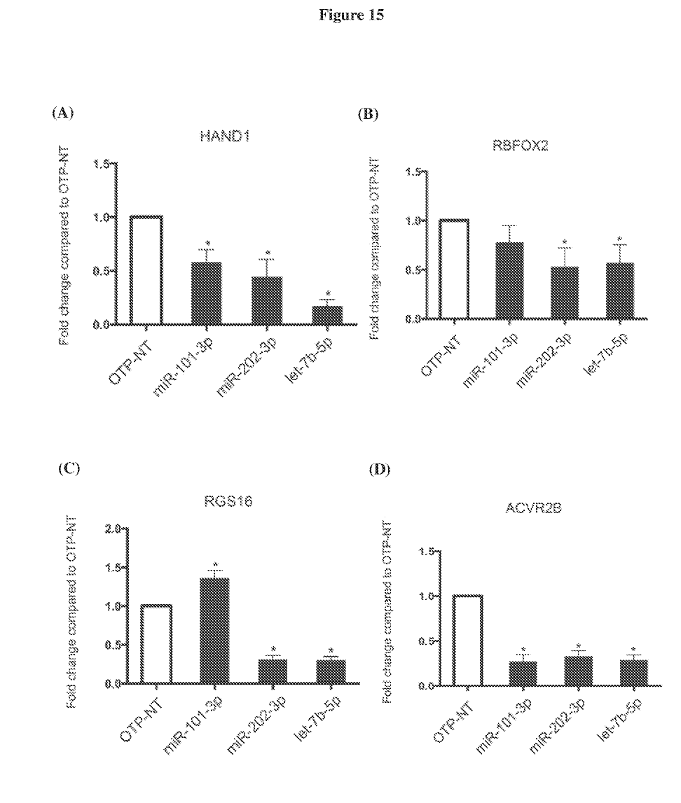

As predicted by in silico analyses using the Moonlitt prediction tool described in Example 5, miRNA overexpression downregulated all of the targets, which the exception that miR-101-3p did not affect mRNA levels for RGS 16 and RBFOX2 (FIG. 15).

The effect of microRNA on levels of target mRNA was then confirmed using luciferase-based reporter assays. The 3′UTR sequences of HAND1 and RGS16 were synthesised by IDT with the corresponding miR binding sites either left as wild type sequence (WT 3′UTR) or mutated to abolish miR binding (MUT 3′UTR). Generated DNA fragments were then cloned in the psiCHECK-2 vector using NotI and XhoI restriction enzymes downstream of hRluc gene (renilla luciferase). The psiCHECK-2 also contained hluc+ gene (firefly luciferase), which enabled signal normalisation to correct for variable transfection efficiency between samples.

To perform the luciferase assay, HEK293T cells were seeded at 2×10⁴ cells per well in a 96-well plate, and the next day, co-transfected with 12.5 ng of the reporter plasmid and 50 nM of the respective microRNA or NT control using Lipofectamine 3000. 24 h after transfection, cells were lysed and subjected to the Dual-Luciferase Reporter 1000 assay (Promega). Briefly, measurements were recorded at 0.5 sec intervals for a total of 26 s with reagent injections of 100 μl of Luciferase Assay Reagent II (firefly luciferase) at 2 s and 100 μl of Stop and Glo Reagent (renilla luciferase) at 14 s. Renilla luciferase activity was normalised to the firefly luciferase signal for a readout of relative microRNA activity. The results were graphed as fold change compared to the NT control. All experiments were performed in biological triplicate.

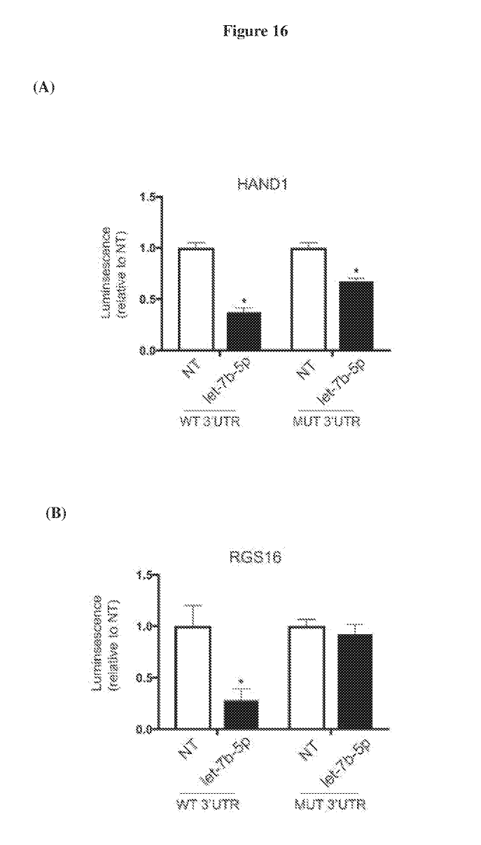

The results of luciferase reporter assays for HAND1 and RGS16 are presented in FIG. 16. Transfection of let-7b-5p significantly reduced the luciferase signal when co-transfected with a vector carrying WT 3′UTR of both HAND1 and RGS16. This effect was completely abolished in the presence of RGS16 3′UTR with a mutated let-7b-5p binding site, and only partially rescued in the presence of the mutated HAND1 3′UTR, suggesting additional binding sites for this microRNA.

To determine whether the four targets have an impact on neuroblastoma survival, KELLY cells were transfected with commercially available siRNAs against HAND1, RGS16, RBFOX2, ACVR2B, MYCN or LIN28B, and cell confluence assays were performed.

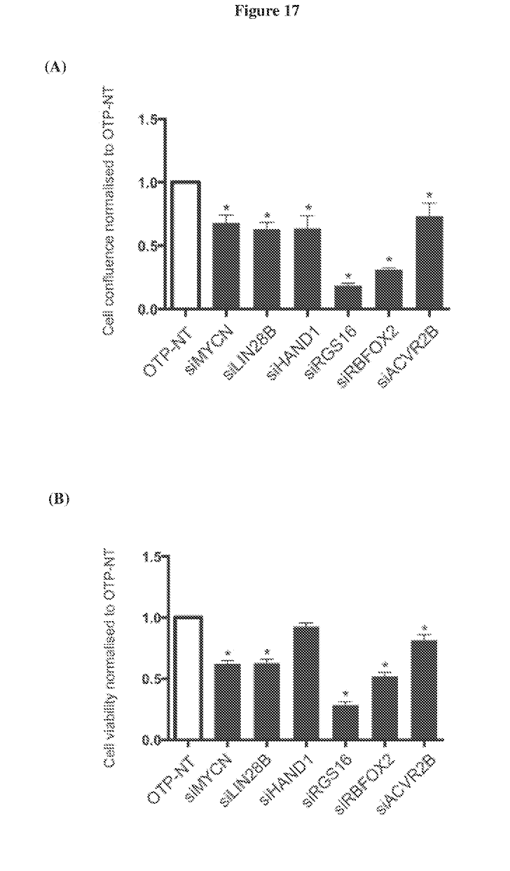

Briefly, KELLY cells were grown in 10% FCS/RPMI and transfected with 40 nM of a siRNA duplex against HAND1, RGS16, RBFOX2, ACVR2B, MYCN, LIN28B or a scrambled control (each obtained from Dharmacon) using Dharmafect 1 (DF1) transfection reagent. The ATP content was measured using a CellTiter-Glo assay (Promega) in accordance with the manufacturer's instructions. Cell confluence and ATP content assays were then performed 96 hours post-transfection to determine viability of cells transfected with siRNA against HAND1, RGS16, RBFOX2 and ACVR2B and this was compared to cells transfected with siRNA against bona fide proto-oncogenes MYCN and LIN28B. Cell confluence was measured using IncuCyte ZOOM, an automated microscopy platform able to take images in regular intervals over the timespan of the experiment. In the present case, images were taken with a 10× objective at 2 hour time intervals for 96 hours following transfection. The IncuCyte ZOOM software was then trained to recognise live cells and exclude the dead cells on a collection of images, after which the cell confluence was calculated automatically in all wells and at each time point over the course of the experiment. FIG. 17B shows cell confluence for KELLY cells transfected with siRNAs against HAND1, RGS16, RBFOX2, ACVR2B, MYCN or LIN28B normalised to cell confluence for KELLY cells transfected with a scrambled control, at the completion of the experiment.

As expected, knockdown of MYCN and LIN28B reduced KELLY cell viability as measured by cell confluence and ATP content, although to a variable degree between independent experiments (FIG. 17A). siRNA knockdown RGS 16 and RBFOX2 led to a dramatic decrease in KELLY cell viability in all biological replicates and assay types compared to the non-targeting control, suggesting that these previously unrecognised factors play a fundamental role in neuroblastoma development.

### Example 8—Identification of Novel miRNAs with a Role in Neuroblastoma Development

To uncover further previously unrecognised miRNAs important for neuroblastoma, the inventors mined the unique set of functional data available in the database described in Example 5.

The inventors queried the database for microRNA families apart from let-7 family that effectively kill KELLY cells carrying MYCN amplification. The most enriched family besides let-7 microRNAs was the miR-515 family. At a very stringent cut-off (normalised viability ≤0.3), six members of the miR-515 family exhibited potent tumour-suppressor effect and were selectively-lethal to neuroblastoma (particularly MYCN-amplified KELLY cells), including miR-516b-5p, miR-517a-3p, miR-517c-3p, miR-518b, miR-519e-5p, and miR-526b-5p. This had not previously been described.

The prediction tool described in Example 5 was then used to investigate the underlying mechanism of their lethality, and a list of ranked high-confidence targets was obtained.

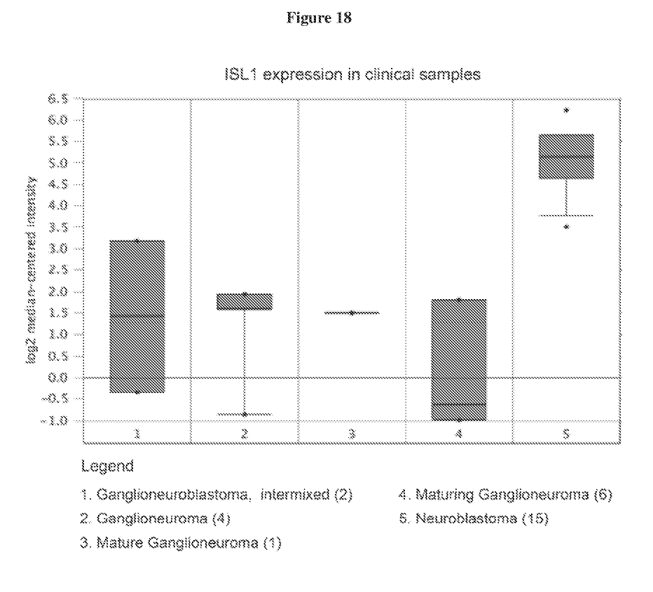

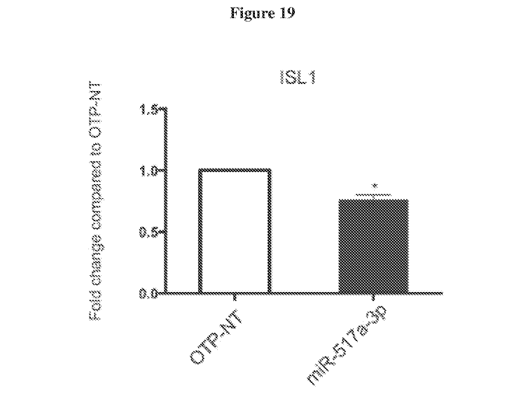

The top candidate target predicted for four of the miRNAs was Isl1, a LIM homeobox transcription factor highly expressed in primary neuroblastoma tumours and cell lines, but not other tumour types (FIG. 18). Importantly, the functional screen performed in Example 1 showed that miR-517a-5p mimic downregulated Isl1 levels in KELLY cells (FIG. 19). In addition, mining of HITS-CLIP experiments (Xue et al., (2013) Cell, 152:82-96) demonstrates direct binding of miR-517a-5p to Isl1 mRNA.

The effect of miR-517a-5p on levels of Isl1 mRNA was then confirmed using luciferase-based reporter assays. The 3′UTR sequences of ISL1 was synthesised by IDT with the corresponding miR binding sites either left as wild type sequence (WT 3′UTR) or mutated to abolish miR binding (MUT 3′UTR). The generated DNA fragment was then cloned in the psiCHECK-2 vector using NotI and XhoI restriction enzymes downstream of hRluc gene (renilla luciferase). The psiCHECK-2 also contained hluc+ gene (firefly luciferase), which enabled signal normalisation to correct for variable transfection efficiency between samples. The luciferase assay was then performed in accordance with the method described in Example 7 above.

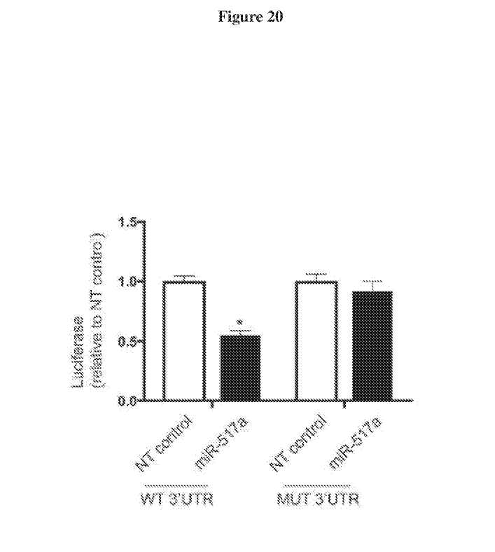

The results of luciferase reporter assay for ISL1 are presented in FIG. 20. Transfection of miR-517a significantly reduced the luciferase signal when co-transfected with a vector carrying WT 3′UTR and this affect was completely abolished in the presence of a vector carrying the 3′UTR with a mutated miR-517a binding site (FIG. 20).

The inventors then sought to determine whether ISL1 plays a role in neuroblastoma survival by performing cell confluence and cell cycle analyses.

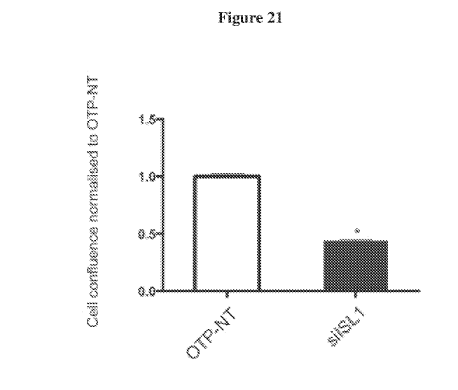

To determine the effect of siRNA against ISL1 on cell confluence, KELLY cells were grown in 10% FCS/RPMI and transfected with 40 nM of a commercially-available siRNA duplex against ISL1 or a scrambled control (both obtained from Dharmacon) using Dharmafect 1 (DF1) transfection reagent in a 6-well plate format. The cell confluence assays were then performed 96 hours post-transfection in accordance with the method described in Example 5. At the completion of the experiment, cell confluence for KELLY cells transfected with siRNA against ISL1 was normalised to cell confluence for KELLY cells transfected with the scrambled control (FIG. 21).

To determine the effect of siRNA against ISL1 on cell cycle, KELLY cells were grown in 10% FCS/RPMI and transfected with 40 nM of a commercially-available siRNA duplex against ISL1 or a scrambled control (both obtained from Dharmacon) using Dharmafect 1 (DF1) transfection reagent in a 6-well plate format. 24 hour post-transfection, media was changed, and 96 h post-transfection, cells were collected and fixed with 70% ethanol at least overnight. The following day, cells were stained with lmg/ml propidium iodide (10 mg/ml final concentration) and RNAse A for up to 5 hours. The cells were then collected using Canto I, and cell cycle profiles were constructed using FlowJo.

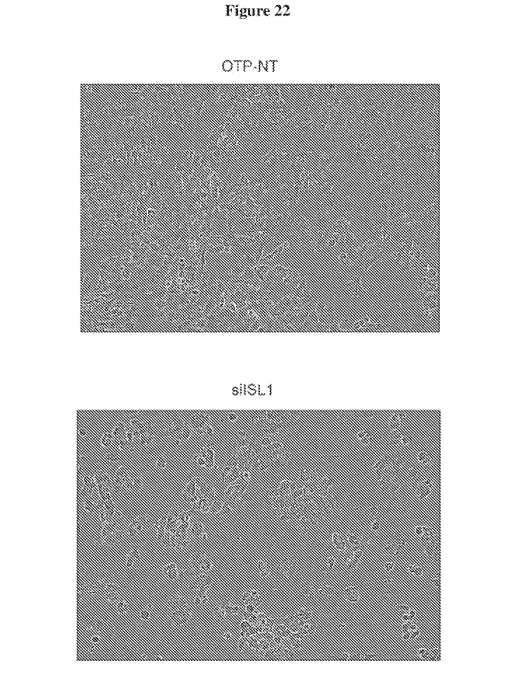

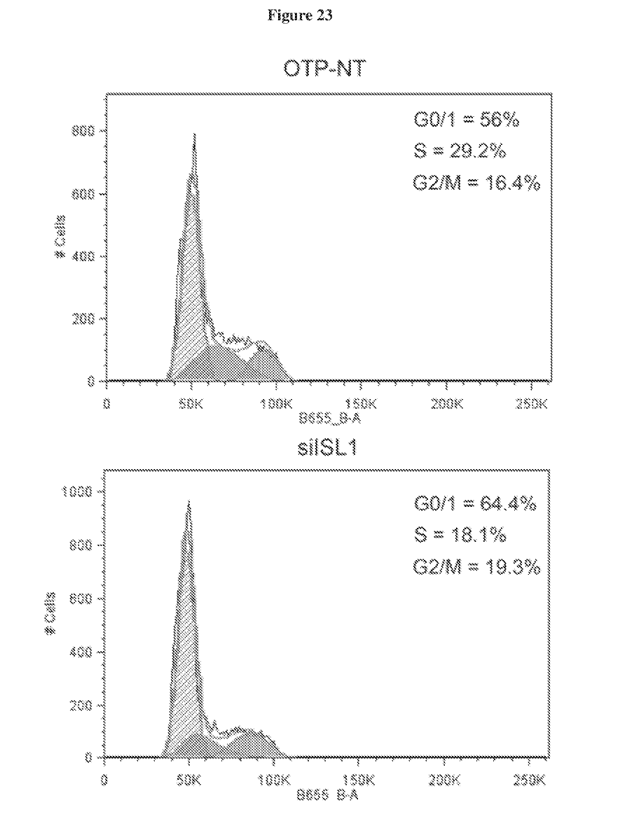

As is apparent from FIG. 21, transfection of KELLY cells with siRNA against ISL1 significantly affected cell growth based on the quantification of cell confluence (FIG. 21 and FIG. 22). ISL1 knockdown also reduced proliferation based on a reduced S-phase fraction in DNA histograms (FIG. 23). When treated with siRNAs against ISL1, Kelly cells dramatically changed their morphology into tightly packed clusters of rounded cells (FIG. 22) suggesting changes in differentiation.

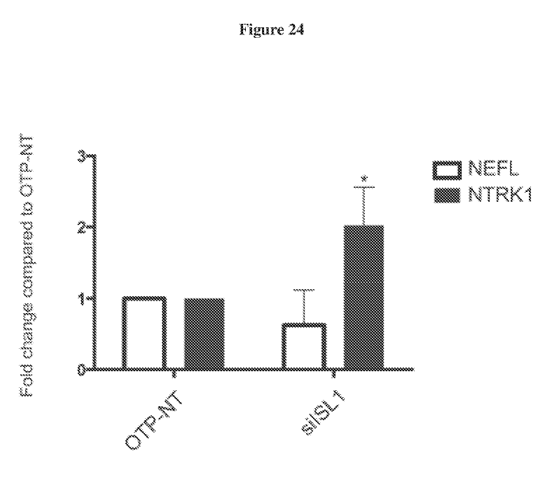

Several differentiation markers of the neuronal lineage previously reported to increase in KELLY cells upon differentiation, including NTRK1 and NEFL, were also measured by qPCR. Briefly, cells were harvested 96 h after transfection and total RNA was extracted using miRneasy kit (Qiagen). RNA was then subjected to reverse transcription to generate cDNA. Quantification of gene expression was then performed using qPCR and Taqman probes and primers for NTRK1 and NEFL. The levels of expression for those genes were normalised to the housekeeping genes B2M and GAPDH. Although the changes of nefl mRNA levels upon ISL1 knockdown were inconsistent between different biological replicates, the absence of ISL1 reproducibly increased the levels of NTRK1, a neurotrophic tyrosine kinase receptor known to mediate neuroblastoma differentiation in different models (Matsushima et al., (1993) Molecular and Cellular Biology, 13:7447-7456; Lavenius et al., (1995) Cell Growth & Differentiation, 6:727-736), suggesting the role for ISL1 in neuroblastoma differentiation (FIG. 24).

### Example 9

Clinical relevance of miRNAs and their putative targets was examined using datasets from neuroblastoma tumours from patients with known outcomes.

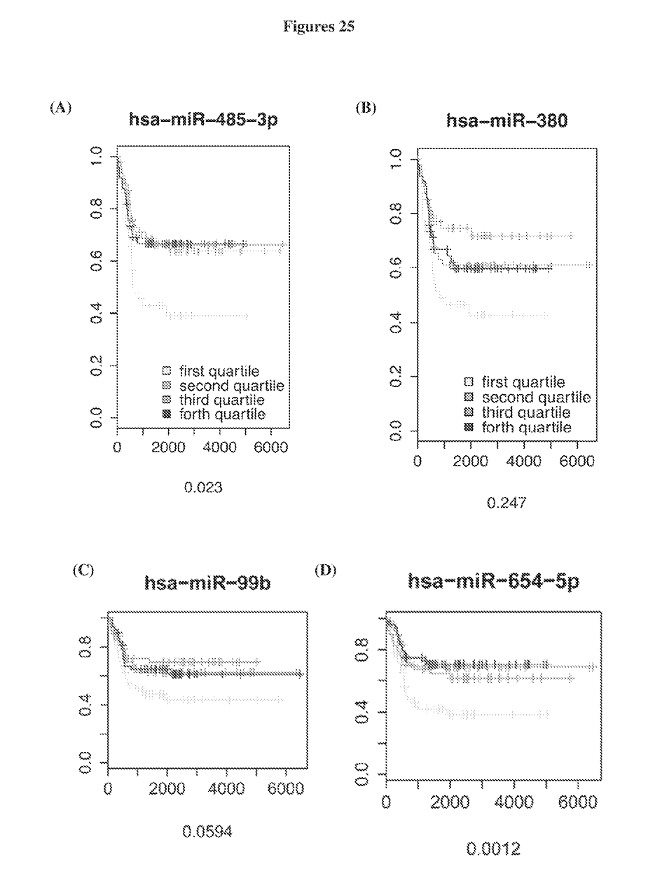

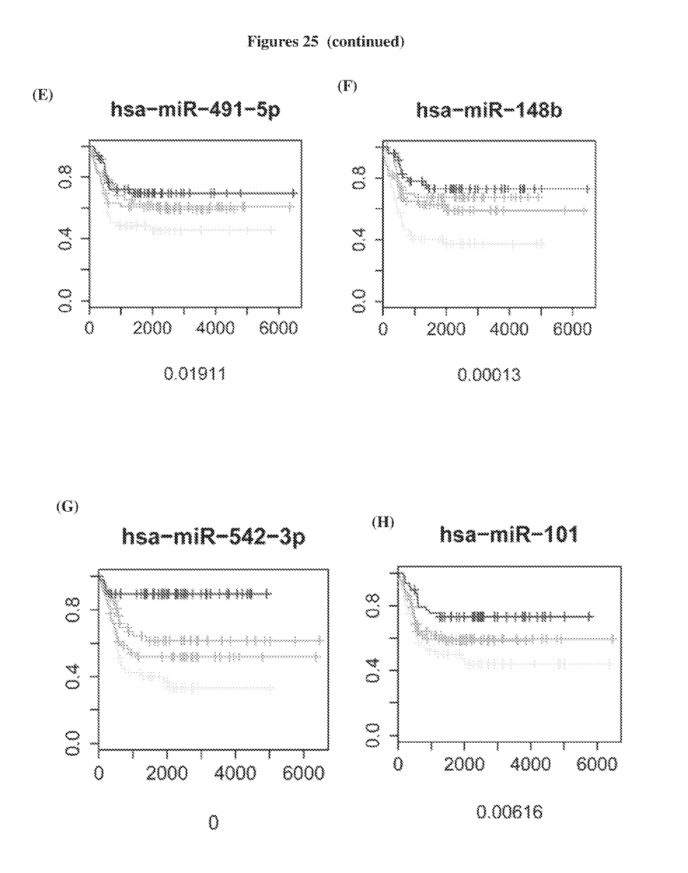

FIG. 25 provides Kaplan-Meier curves of event-free survival for three synthetic lethal and five lethal miRNAs identified in Example 3, generated from an unpublished cohort of about 200 neuroblastoma patients. Yellow line represents the quartile of patients with lowest expression of the given miRNA. This data supports the hypothesis that these miRNAs are tumour suppressor miRNAs and that increasing their expression may improve patient outcomes.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

It is to be understood that any discussion of public documents, acts, materials, devices, articles or the like included herein is solely for the purpose of providing a context for the present invention. It is not to be taken as an admission that any or all of these matters were common general knowledge in the field relevant to the present invention as it existed before the priority date of any claim of this application.

What is claimed:

1. A method for treating neuroblastoma in a subject suffering therefrom, said method comprising administering to the subject an RNA interference (RNAi) molecule selected from the group consisting of:
(i) a microRNA (miRNA) selected from the group of miRNAs set forth in Table 1 or Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

2. The method according to claim 1, wherein the RNAi molecule is selected from the group consisting of:
(i) a miRNA selected from the group of miRNAs set forth in Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

3. The method according to claim 1, wherein the RNAi molecule is selected from the group consisting of:
(i) a miRNA selected from the group of miRNAs set forth in rows 1-20 of Table 1;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

4. The method according to claim 2, wherein the RNAi molecule is a miRNA selected from the group consisting of miRNAs from clause (i).

5. The method according to claim 1, wherein the RNAi molecule is selected from the group consisting of:
(i) a miRNA selected from the group of miRNAs set forth in Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

6. The method according to claim 1, wherein the RNAi molecule is selected from the group consisting of:
(i) a miRNA selected from the group of miRNAs set forth in rows 1-13 of Table 2;
(ii) a miRNA comprising an effector sequence of a miRNA at (i);
(iii) a miRNA comprising an effector sequence which targets the same mRNA transcript sequence as an effector sequence of a miRNA at (i);
(iv) a primary miRNA (pri-miRNA) corresponding to a miRNA of any one of (i) to (iii);
(v) a precursor miRNA (pre-miRNA) corresponding to a miRNA any one of (i) to (iii);
(vi) a miRNA mimic corresponding to a miRNA of any one of (i) to (iii);
(vii) a miRNA of any one of (i) to (iii) which has a chemical modification or a modified nucleobase; and
(viii) a nucleic acid encoding an RNAi molecule of any one of (i)-(v).

7. The method according to claim 5, wherein the RNAi molecule is a miRNA selected from the group consisting of miRNAs from clause (i).

8. The method according to claim 1, wherein administration of the RNAi molecule sensitizes a neuroblastoma cell to a chemotherapeutic agent.

9. The method according to claim 1, wherein administration of the RNAi molecule reduces a therapeutically effective dose of a chemotherapeutic agent for treating the neuroblastoma.

10. The method according to claim 9, wherein the therapeutically effective dose of the chemotherapeutic agent for treating neuroblastoma is reduced relative to a dose of the chemotherapeutic agent which is therapeutically effective in a subject who has not been, or will not be, administered the RNAi molecule.

11. The method according to claim 1, wherein the nucleic acid encoding the RNAi molecule is comprised within an expression vector.

12. The method according to claim 11, wherein the expression vector is a plasmid or viral particle.

13. The method according to claim 1, wherein the RNAi molecule is comprised within an exosome or microvesicle.

14. The method according to claim 1, further comprising administering to the subject a chemotherapeutic agent.

15. The method according to claim 14, wherein the chemotherapeutic agent is administered at a reduced therapeutically effective dose relative to a dose of the chemotherapeutic agent which is therapeutically effective in a subject who has not been, or will not be, administered the RNAi molecule.

16. The method according to claim 14, wherein the chemotherapeutic agent and the RNAi molecule are administered together, concurrently or sequentially.

17. The method according to claim 1, wherein the subject has previously received treatment with a chemotherapeutic agent.

18. The method according to claim 8, wherein the neuroblastoma is refractory to treatment with the chemotherapeutic agent in the absence of adjunctive treatment with the RNAi molecule or is a recurrent neuroblastoma.

19. The method according to claim 8, wherein the chemotherapeutic agent is a vinca alkaloid or an anthracycline.

20. The method according to claim 19, wherein the anthracycline is selected from the group consisting of daunorubicin, doxorubicin, epirubicin, idarubicin and mitoxantrone.

21. The method according to claim 19, wherein the vinca alkaloid is selected from the group consisting of vincristine, vinblastine, vinorelbine and vindesine.
